(19) United States
(12) Patent Application Publication
(10) Pub. No.: US 2015/0249224 A1
(43) Pub. Date: Sep. 3, 2015

(54) NOVEL COMBINATION OF A HOST COMPOUND AND A DOPANT COMPOUND AND AN ORGANIC ELECTROLUMINESCENCE DEVICE COMPRISING THE SAME

(71) Applicant: Rohm and Haas Electronic Materials Korea Ltd., Cheonan (KR)

(72) Inventors: So-Young Jung, Hwaseong (KR); Bong-Ok Kim, Seoul (KR); Chi-Sik Kim, Hwaseong (KR); Hyun Kim, Suwon (KR); Jong-Seok Ku, Hwaseong (KR); Hyuck-Joo Kwon, Cheonan (KR); Kyung-Joo Lee, Seoul (KR); Kyoung-Jin Park, Seongnam (KR)

(73) Assignee: ROHM AND HAAS ELECTRONIC MATERIALS KOREA LTD., Cheonan (KR)

(21) Appl. No.: 14/426,173

(22) PCT Filed: Sep. 10, 2013
(86) PCT No.: PCT/KR2013/008178

§ 371 Date: Mar. 5, 2015

(30) Foreign Application Priority Data
Sep. 11, 2012 (KR) 10-2012-0100533

(51) Int. Cl. H01L 51/00; C07D 495/04; C07D 403/14; C09K 11/06; C07D 403/04; C07D 409/14; C07D 487/04; C07D 407/14; C07F 15/00; C07D 491/048

(52) U.S. Cl. CPC H01L 51/0085; C07F 15/0033; C07D 495/04; C07D 403/14; C07D 491/048; C07D 403/04; C07D 409/14; C07D 487/04; C07D 407/14; C09K 11/06; H01L 51/0072; H01L 51/0067; H01L 51/0071; H01L 51/0052; H01L 51/0074; H01L 51/0073; H01L 51/0058; C09K 2211/185; C09K 2211/1018; H01L 51/5012

(57) ABSTRACT

The present invention relates to a specific combination of a dopant compound and a host compound, and an organic electroluminescent device comprising the same. The organic electroluminescent device according to the present invention, has an advantage of showing a higher luminous efficiency under a driving voltage lower than that of the device comprising conventional luminescent materials.

### TECHNICAL FIELD

The present invention relates to a novel combination of a host compound and a dopant compound and an organic electroluminescence device comprising the same.

### BACKGROUND ART

An electroluminescent (EL) device is a self-light-emitting device which has advantages in that it provides a wider viewing angle, a greater contrast ratio, and a faster response time compared to LCDs. An organic EL device was first developed by Eastman Kodak, by using small aromatic diamine molecules, and aluminum complexes as materials for forming a light-emitting layer [Appl. Phys. Lett. 51, 913, 1987].

The most important factor determining luminous efficiency in an organic EL device is the light-emitting material. The electroluminescent material includes a host material and a dopant material for purposes of functionality. Typically, a device that has much superior electroluminescent properties is known to have a structure in which a host is doped with a dopant to form an electroluminescent layer. Recently, the development of an organic EL device having high efficiency and long lifespan is being urgently called for. Particularly, taking into consideration the electroluminescent properties required of medium to large OLED panels, the development of materials much superior to conventional electroluminescent materials is urgent.

Until now, fluorescent materials have been widely used as a light-emitting material. However, in view of electroluminescent mechanisms, developing phosphorescent materials is one of the best methods to theoretically enhance luminous efficiency by four (4) times. Iridium(III) complexes have been widely known as dopant compounds of phosphorescent substances, including bis(2-(2′-benzothienyl)-pyridinato-N,C3′)iridium(acetylacetonate) [(acac)Ir(btp)₂], tris(2-phenylpyridine)iridium [Ir(ppy)₃] and bis(4,6-difluorophenylpyridinato-N,C2)picolinato iridium [Firpic] as red, green and blue materials, respectively. Until now, 4,4′-N,N′-dicarbazol-biphenyl (CBP) was the most widely known host material for phosphorescent substances. Further, an organic EL device of high efficiency using bathocuproine (BCP) and aluminum(III)bis(2-methyl-8-quinolinate)(4-phenylphenolate) (BAlq) for a hole blocking layer is also known. However, there were problems in power efficiency, operational life span, and luminous efficiency, when applying a light-emitting material comprising conventional dopant and host compounds.

Korean Patent Appln. Laying-Open No. KR 2007-050438 A discloses iridium complexes introducing an alkyl or an aryl group to an Ir(ppy)₃ structure, which is a conventional dopant compound, as a dopant compound comprised in a light-emitting material of an organic electroluminescent device. However, the above reference does not disclose a preferable combination with a host compound, and still could not solve the problems of luminous efficiency, etc.

The present inventors found that a specific combination of a dopant compound and a host compound is suitable for manufacturing organic EL devices having high color purity, high luminance, and a long lifespan.

### DISCLOSURE OF THE INVENTION

### Problems to be Solved

The objective of the present invention is to provide a novel dopant and host combination, and an organic electroluminescent device comprising the same which provides excellent luminous efficiency in lowered operating voltages.

### Solution to Problems

In order to achieve said purposes, the present invention provides a combination of one or more dopant compounds represented by the following formula 1, and one or more host compounds represented by the following formula 2:

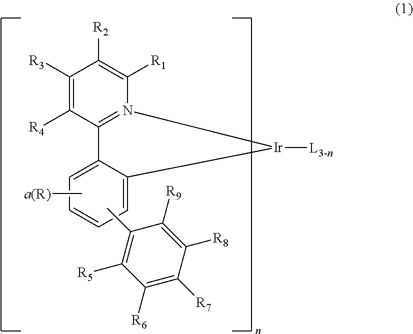

wherein

L is an organic ligand;

R₁ to R₉ each independently represent hydrogen, deuterium, a halogen, a substituted or unsubstituted (C1-C30)alkyl, a substituted or unsubstituted (C3-C30)cycloalkyl, a substituted or unsubstituted (C1-C30)alkoxy, a substituted or unsubstituted (C6-C30)aryl, or a substituted or unsubstituted 3- to 30-membered heteroaryl;

R represents hydrogen, a halogen, a substituted or unsubstituted (C1-C30)alkyl, or a substituted or unsubstituted (C3-C30)cycloalkyl;

a represents an integer of 1 to 3; where a is an integer of 2 or more, each of R are same or different; and

n represents an integer of 1 to 3;

A-D-C (2)

wherein

D represents a single bond, a substituted or unsubstituted (C3-C30)cycloalkylene, a substituted or unsubstituted (C6-C30)arylene, or a substituted or unsubstituted 5- to 30-membered heteroarylene;

A is represented by the following formula 3 or 4:

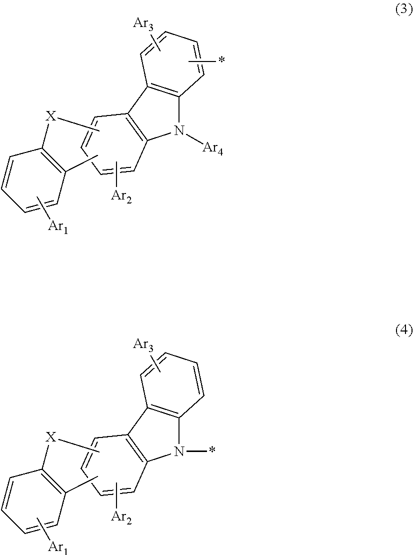

wherein

-* represents a position to which D is linked;

X represents NR₁₀, O, S, or CR₁₁R₁₂;

Ar₁ to Ar₄ each independently represent hydrogen, a substituted or unsubstituted (C1-C30)alkyl, a substituted or unsubstituted (C6-C30)aryl, a substituted or unsubstituted 5- to 30-membered heteroaryl, a substituted or unsubstituted (C3-C30)cycloalkyl, —NR₁₃R₁₄, —SiR₁₅R₁₆R₁₇, —SR₁₈, or —OR₁₉; and

R₁₀ to R₁₉ each independently represent hydrogen, a substituted or unsubstituted (C1-C30)alkyl, or a substituted or unsubstituted (C6-C30)aryl;

C is represented by the following formula 5:

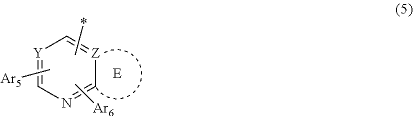

wherein

-* represents a position to which D is linked;

Y and Z each independently represent CH or N;

E ring represents a substituted or unsubstituted benzene ring or is absent;

Ar₅ and Ar₆ each independently represent hydrogen, a substituted or unsubstituted (C6-C30)aryl, a substituted or unsubstituted 5- to 30-membered heteroaryl, a substituted or unsubstituted (C6-C30)cycloalkyl, —NR₂₁R₂₂, —SiR₂₃R₂₄R₂₅, —SR₂₆, or —OR₂₇; and

R₂₁ to R₂₇ each independently represent hydrogen, a substituted or unsubstituted (C1-C30)alkyl, a substituted or unsubstituted (C6-C30)aryl, or a substituted or unsubstituted 5- to 30-membered heteroaryl.

### Effects of the Invention

The organic electroluminescent device according to the present invention contains a specific combination of a dopant compound and a host compound, and provides an advantage of showing a higher luminous efficiency under a driving voltage lower than that of the device comprising conventional luminescent materials.

### EMBODIMENTS OF THE INVENTION

Hereinafter, the present invention will be described in detail. However, the following description is intended to explain the invention, and is not meant in any way to restrict the scope of the invention.

The present invention relates to an organic electroluminescent device comprising one or more dopant compounds represented by formula 1, and one or more host compounds represented by formula 2.

The dopant compound represented by formula 1 is preferably represented by formula 6 or 7:

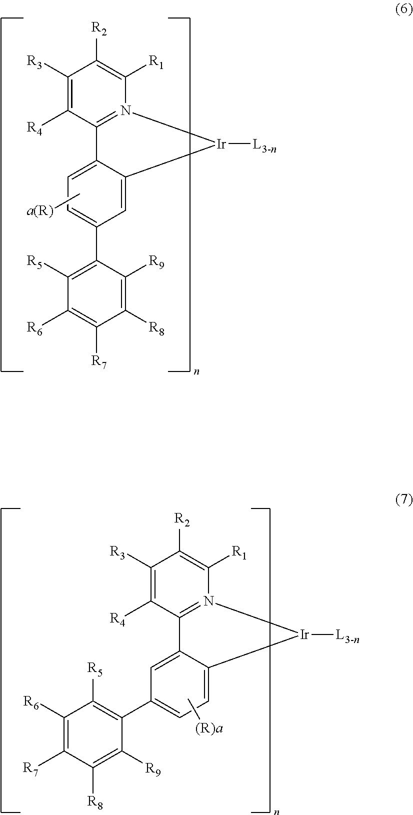

wherein R, R₁ to R₉, L, n and a are as defined in formula 1.

In formulae 1, 6, and 7, L is selected from the group consisting of the following structures, but are not limited thereto.

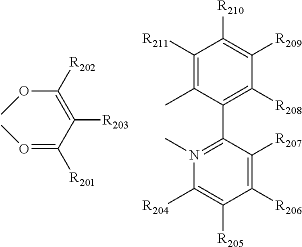

wherein R₂₀₁ to R₂₁₁ each independently represent hydrogen, deuterium, a halogen, a substituted or unsubstituted (C1-C30)alkyl, or a substituted or unsubstituted (C3-C30)cycloalkyl.

In formulae 1, 6, and 7, R, and R₁ to R₉ preferably each independently represent hydrogen, a halogen, a substituted or unsubstituted (C1-C10)alkyl, or a substituted or unsubstituted (C3-C10)cycloalkyl, and more preferably each independently represent hydrogen, a halogen, a (C1-C6)alkyl unsubstituted or substituted with a (C1-C6)alkyl, or a (C3-C7)cycloalkyl unsubstituted or substituted with a (C1-C6)alkyl.

The representative compounds of formula 1 include the following compounds, but are not limited thereto:

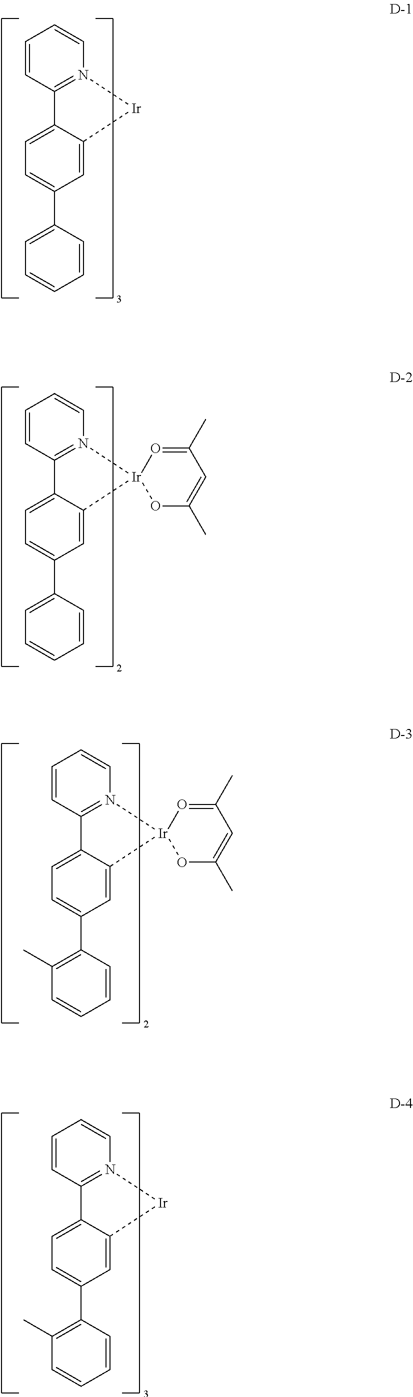

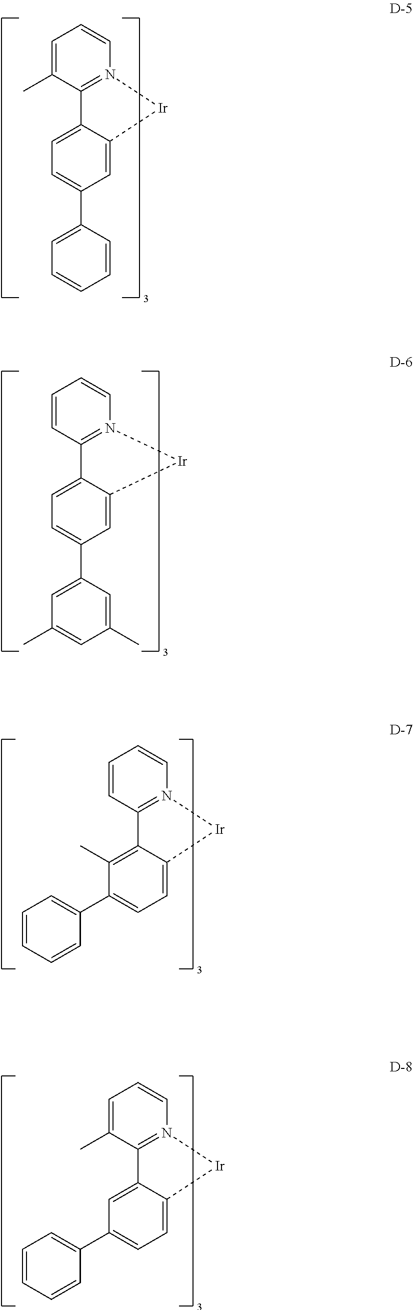

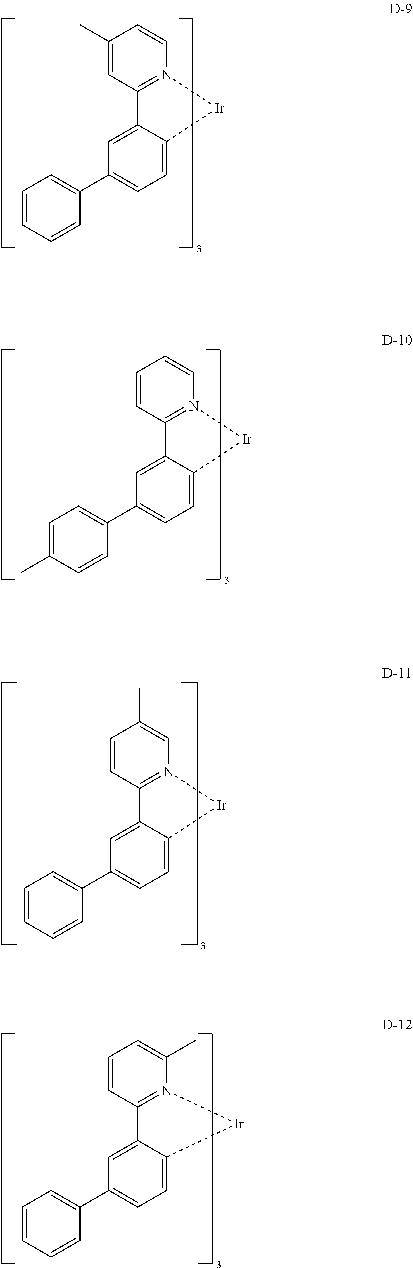

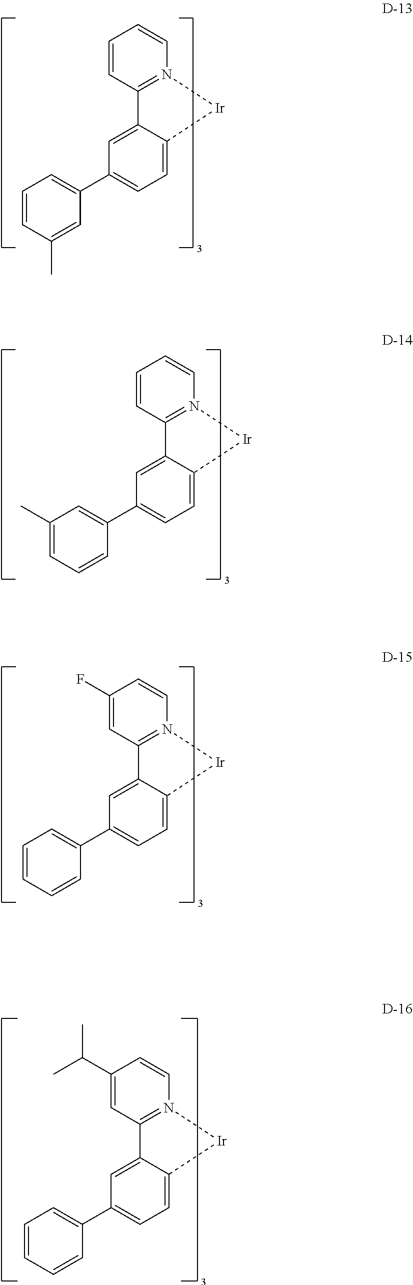

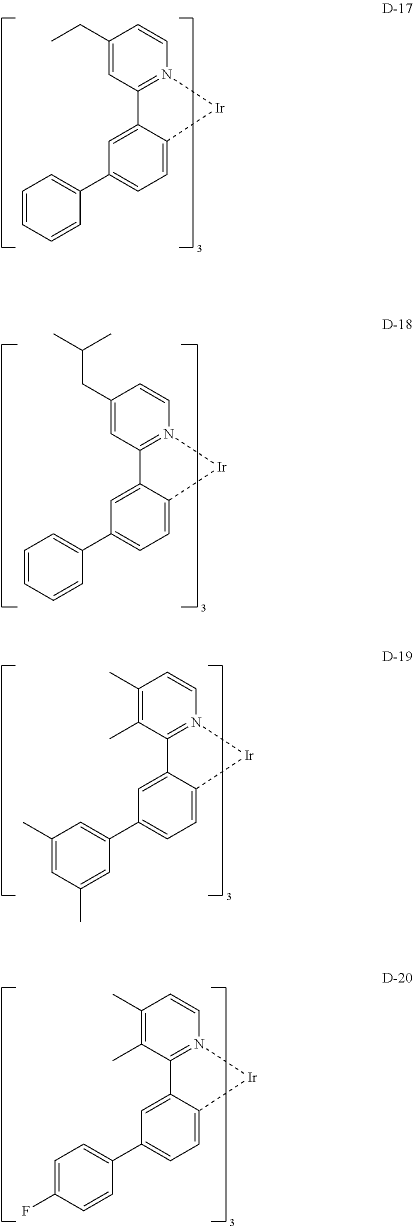

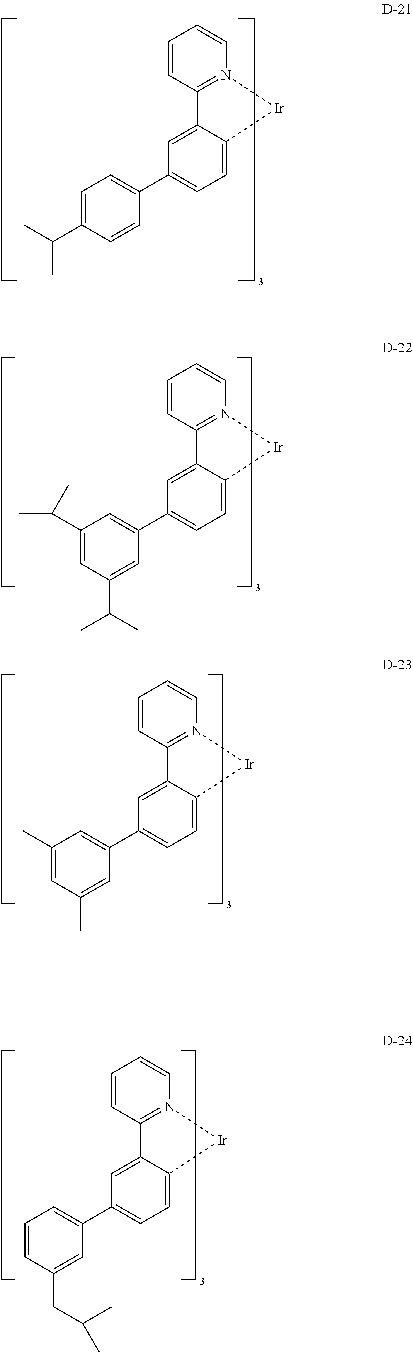

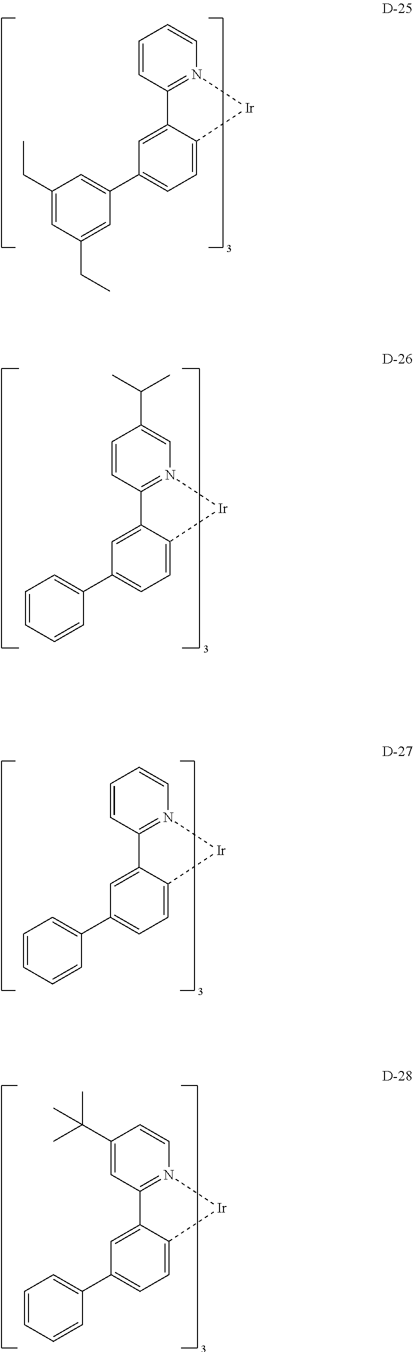

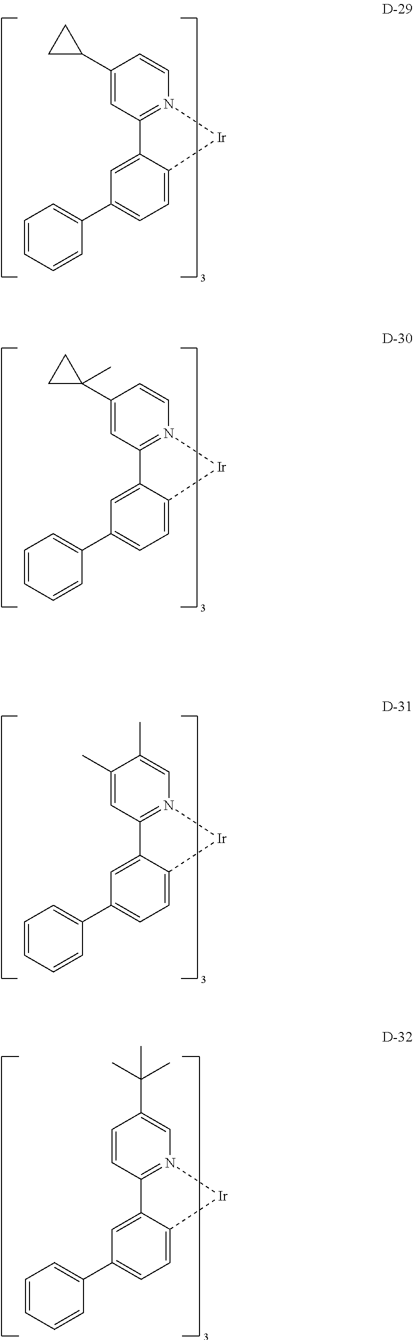

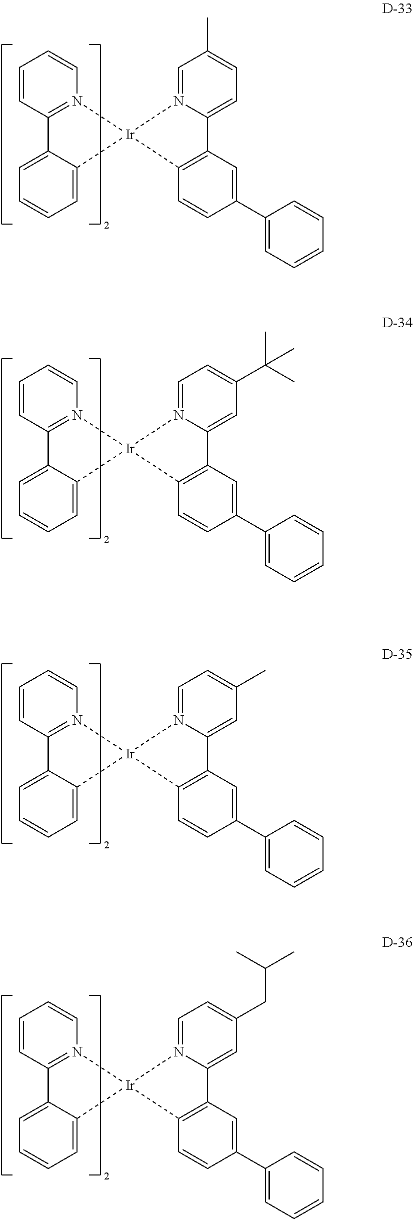

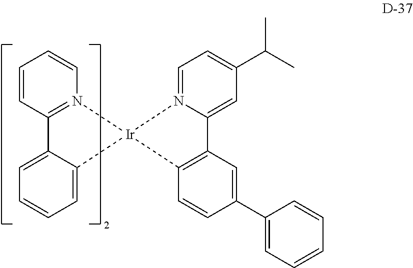

In formula 2, D preferably represents a single bond, or is selected from the group consisting of:

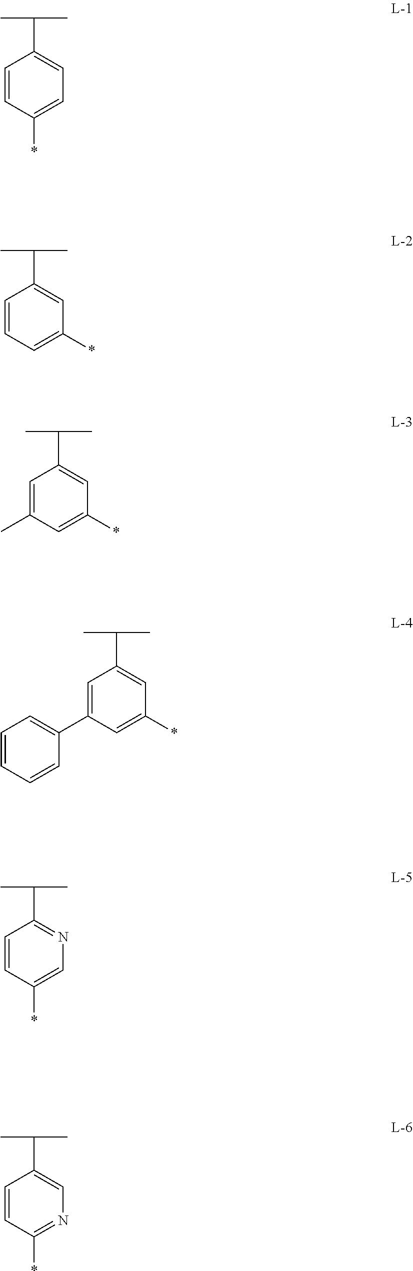

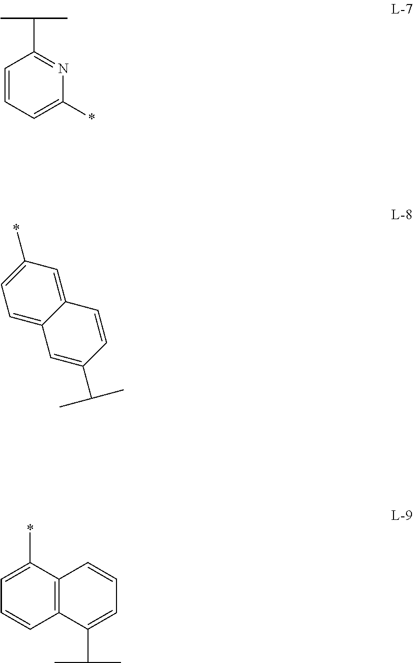

wherein -* represents a position to which C is linked, and [symbol] represents a position to which A is linked.

In formula 2, A is represented by formula 3 or 4, wherein X preferably represents NR₁₀, O, S, or CR₁₁R₁₂, and R₁₀ to R₁₂ preferably each independently represent a (C1-C6)alkyl, or a (C6-C12)aryl. Ar₁ to Ar₄ preferably each independently represent hydrogen; a (C1-C10)alkyl unsubstituted or substituted with a (C1-C6)alkyl; a (C6-C15)aryl unsubstituted or substituted with a (C1-C6)alkyl or a (C6-C12)aryl; a 5- to 15-membered heteroaryl unsubstituted or substituted with a (C1-C6)alkyl or a (C6-C12)aryl; or —SiR₁₅R₁₆R₁₇, wherein R₁₅ to R₁₇ preferably each independently represent a (C1-C6)alkyl.

In formula 2, A is preferably selected from the group consisting of:

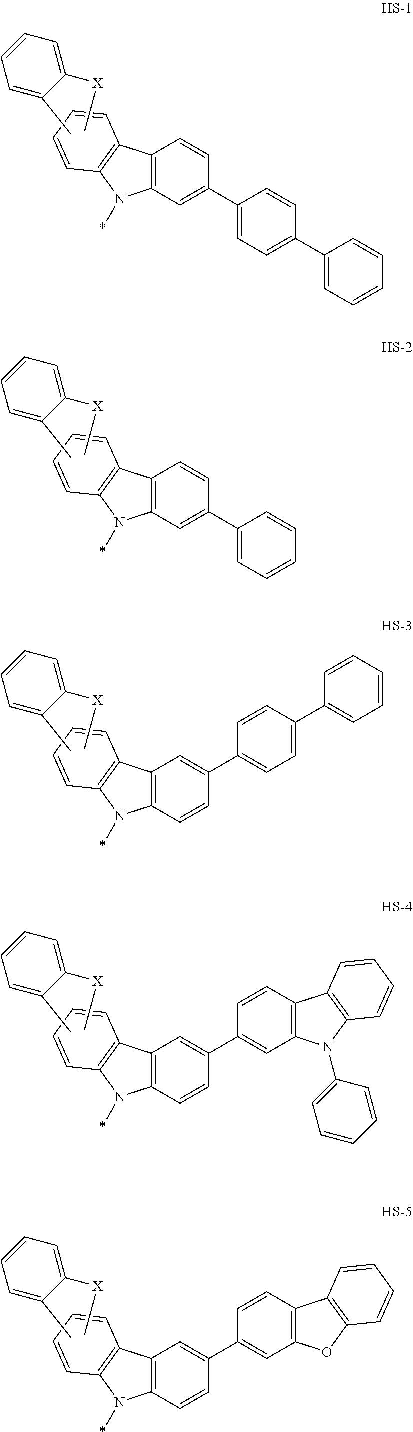

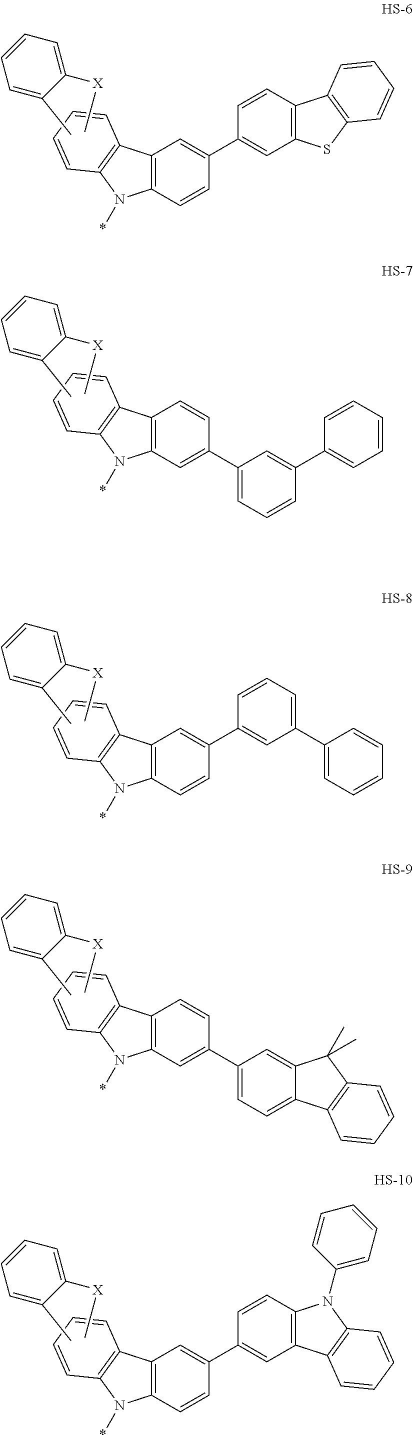

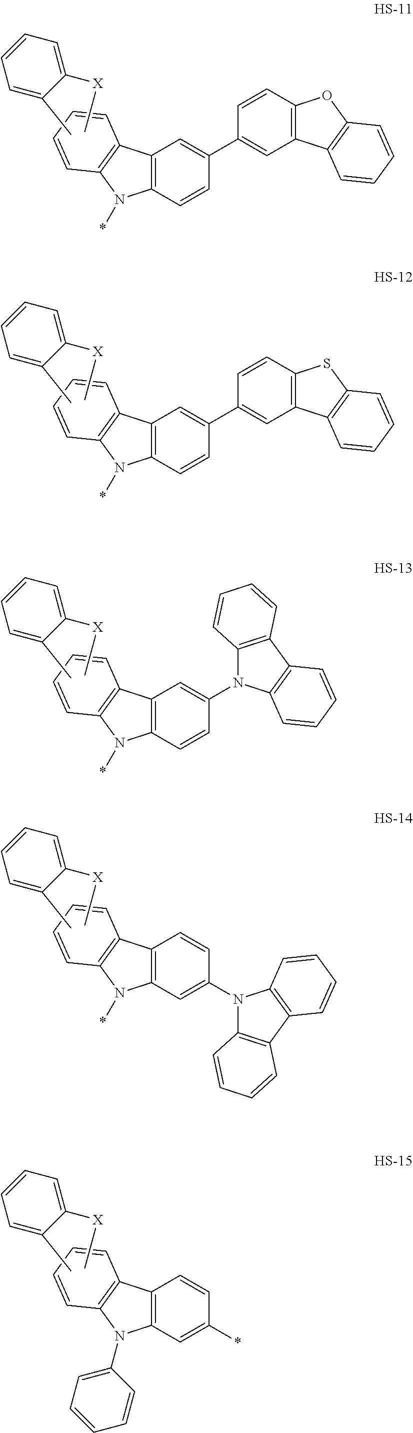

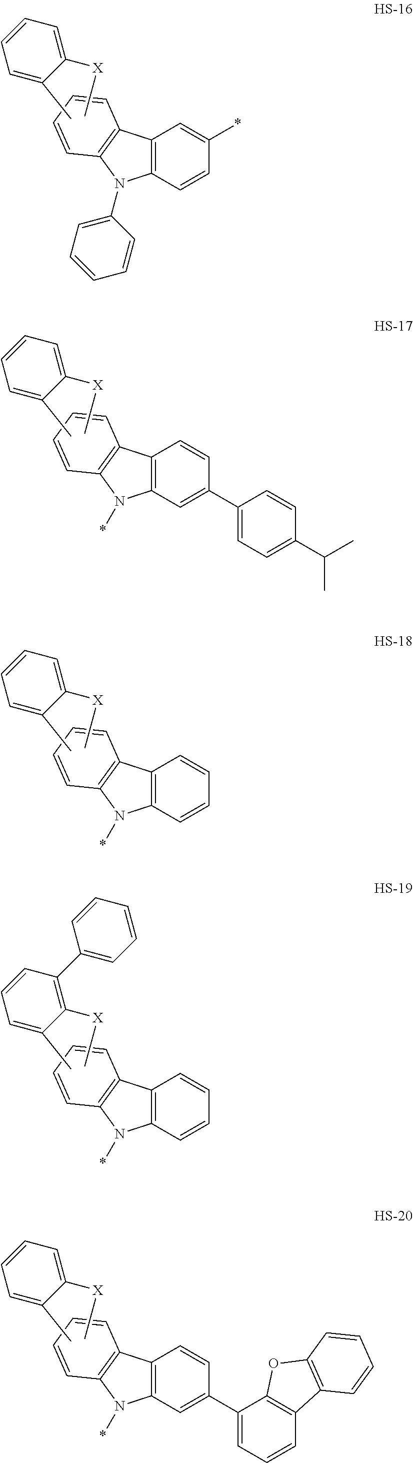

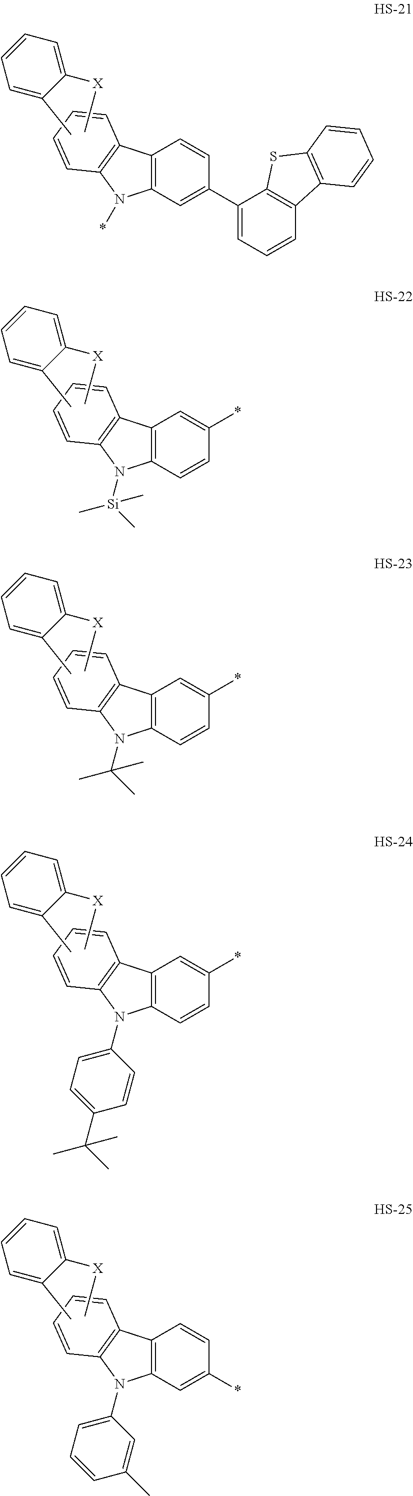

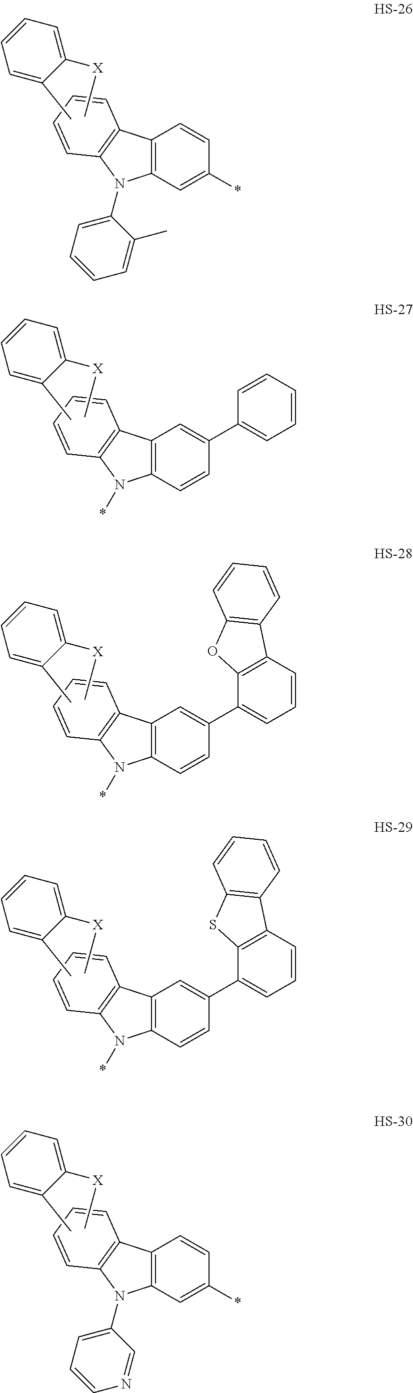

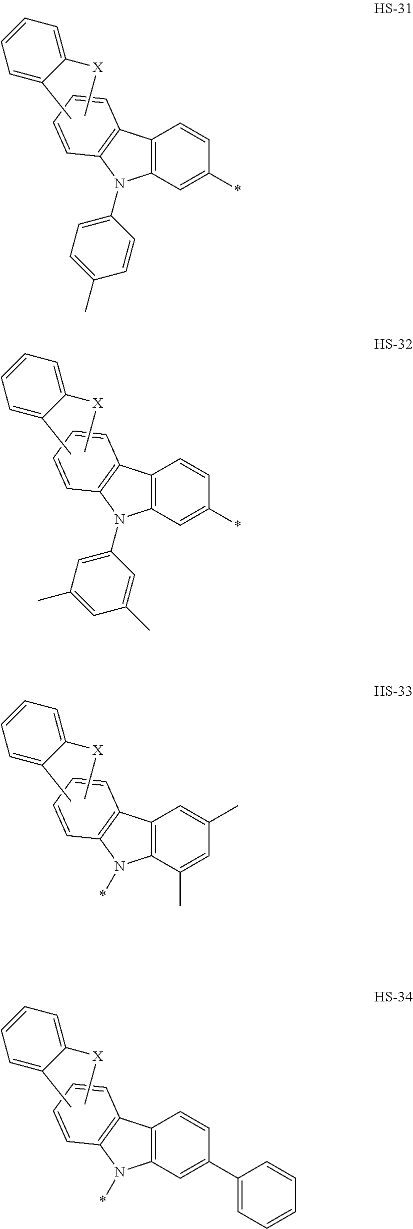

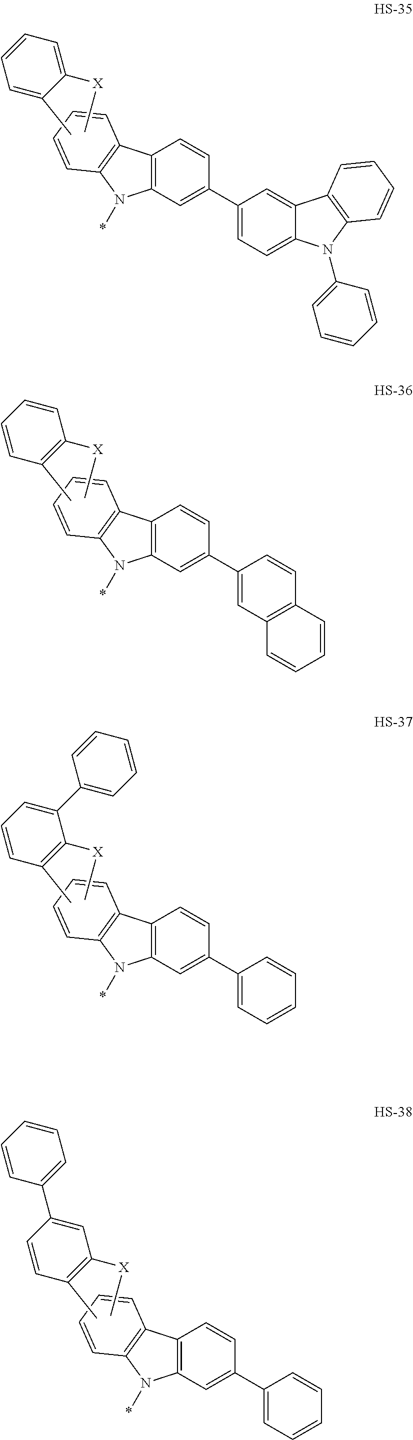

In formula 2, C is represented by formula 5, wherein E ring represents a benzene ring unsubstituted or substituted with a (C6-C12)aryl, or is absent, Ar₅ and Ar₆ preferably each independently represent hydrogen, a substituted or unsubstituted (C6-C20)aryl, a substituted or unsubstituted 5- to 20-membered heteroaryl, or —SiR₂₃R₂₄R₂₅, and R₂₃ to R₂₅ each independently represent a (C6-C12)aryl.

The substituents of the substituted aryl, and the substituted heteroaryl each independently are preferably at least one selected from the group consisting of a halogen, a (C1-C6)alkyl, a (C3-C7)cycloalkyl, a (C6-C12)aryl unsubstituted or substituted with a (C1-C6)alkyl, a 5- to 15-membered heteroaryl unsubstituted or substituted with a (C6-C12)aryl, a tri(C6-C12)arylsilyl, and a (C1-C6)alkyldi(C6-C12)arylsilyl.

In formula 2, C is preferably selected from the group consisting of:

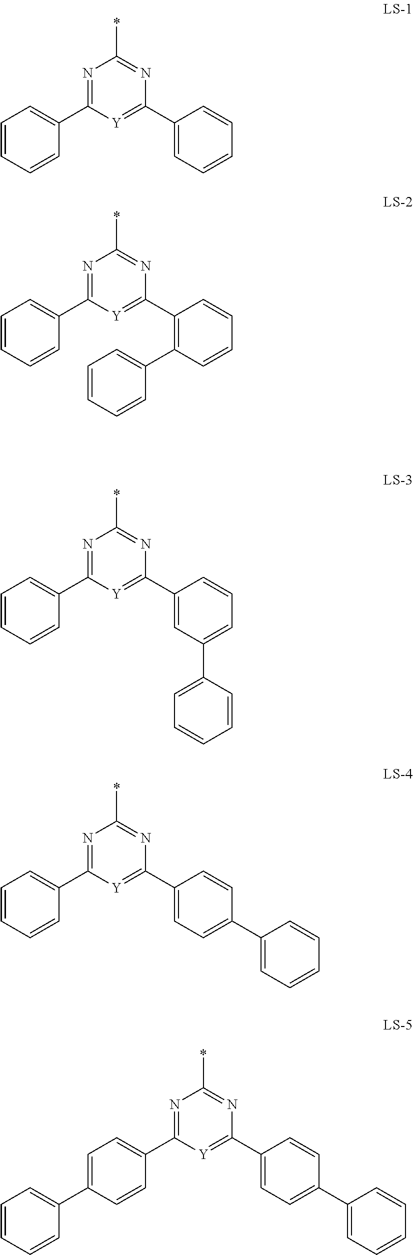

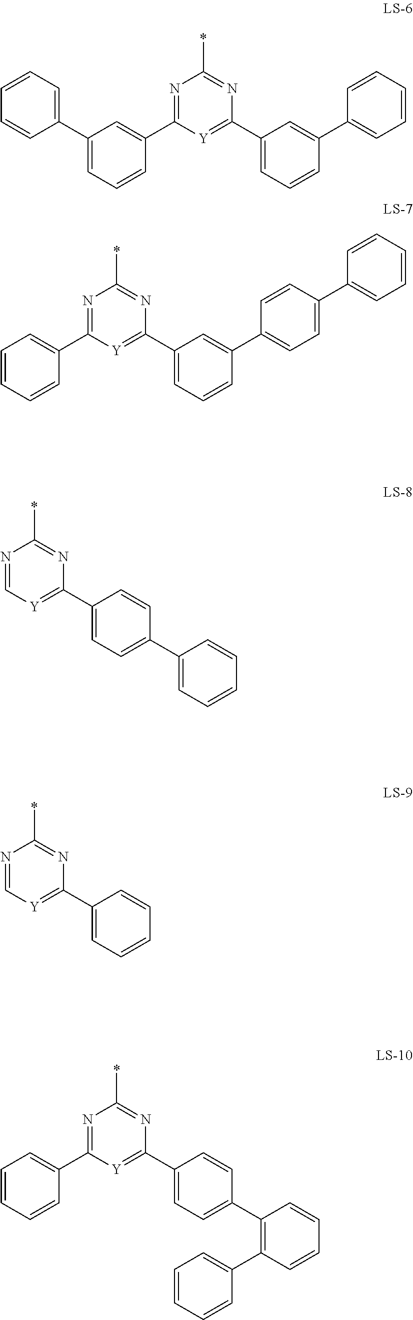

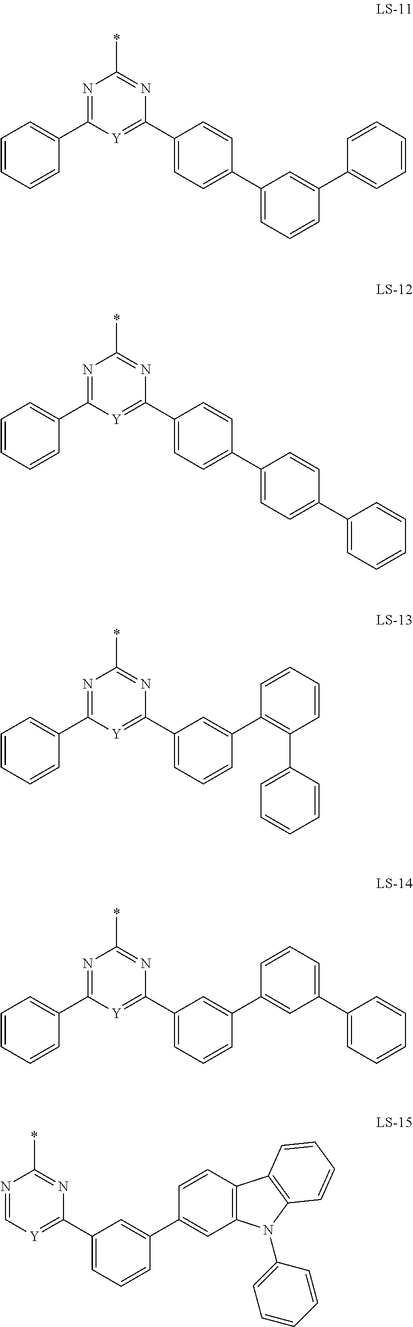

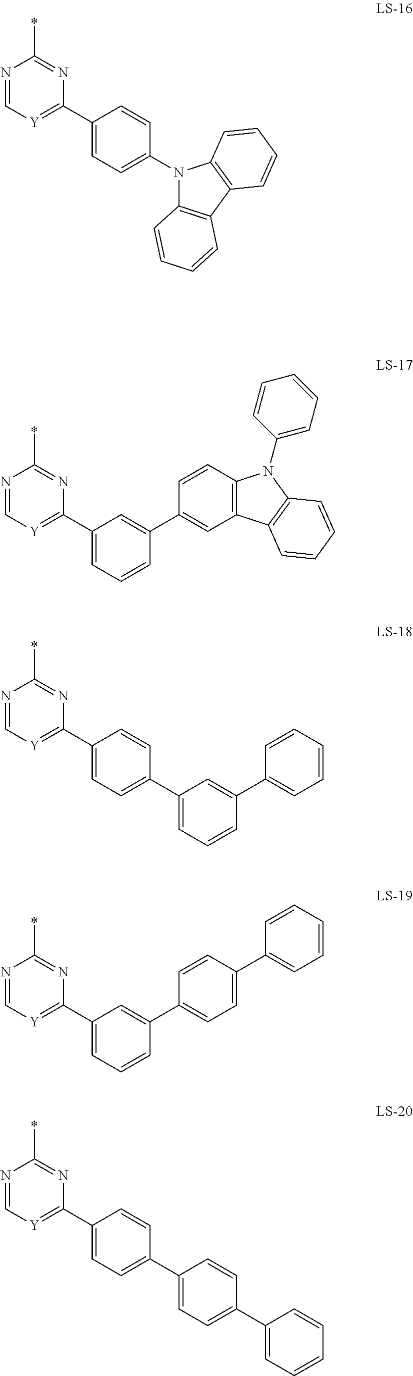

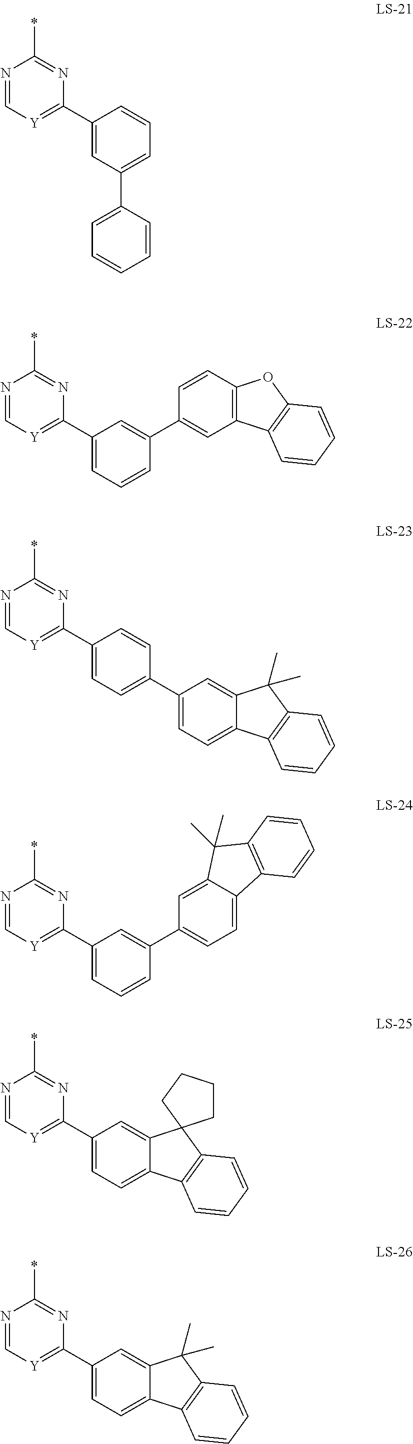

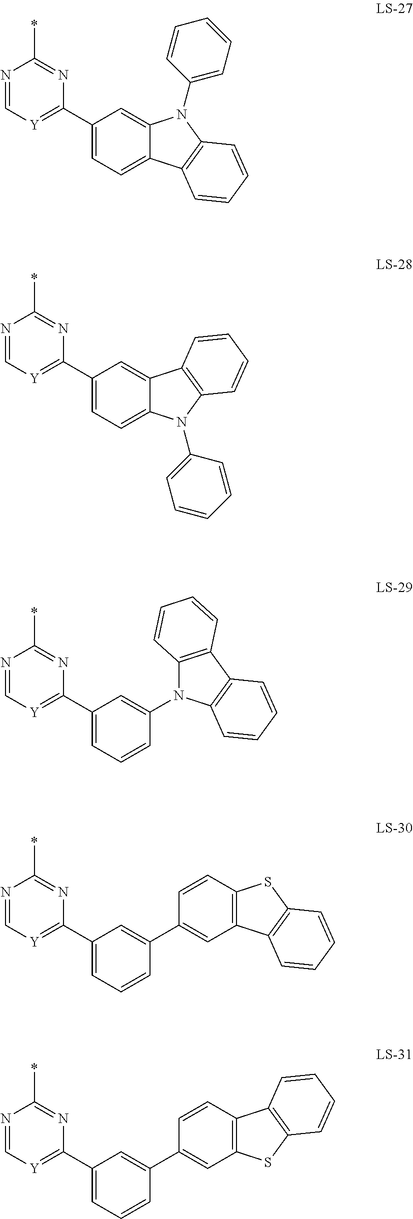

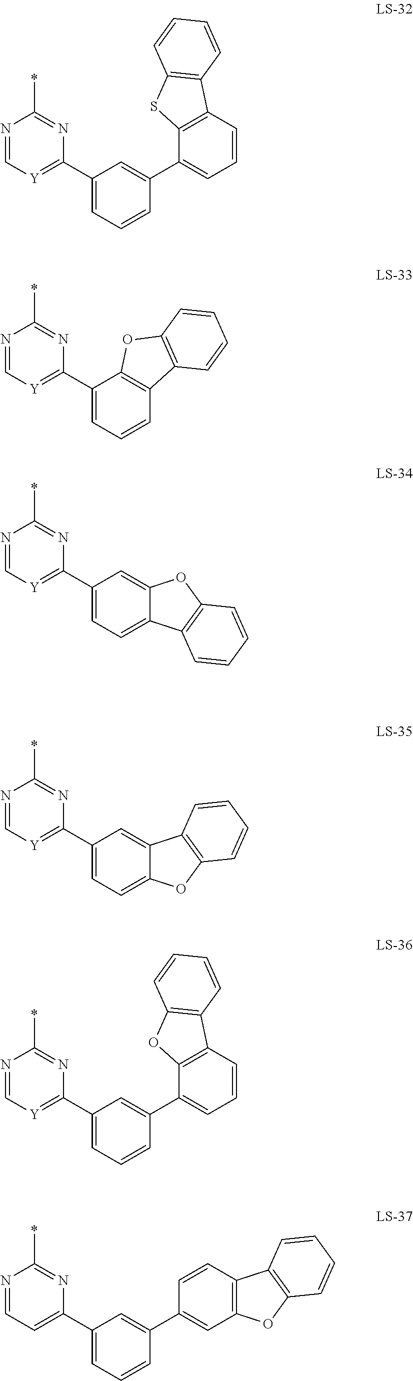

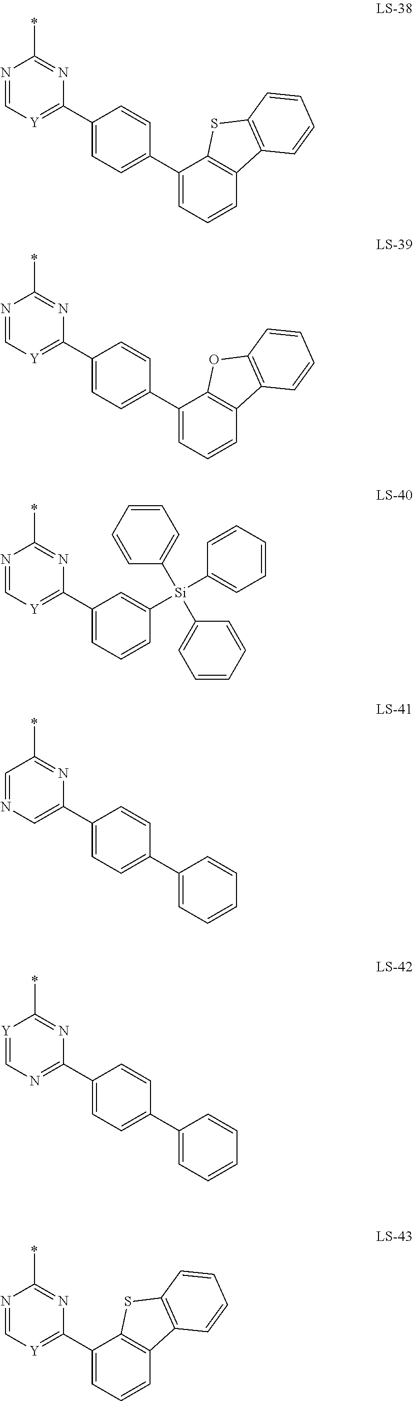

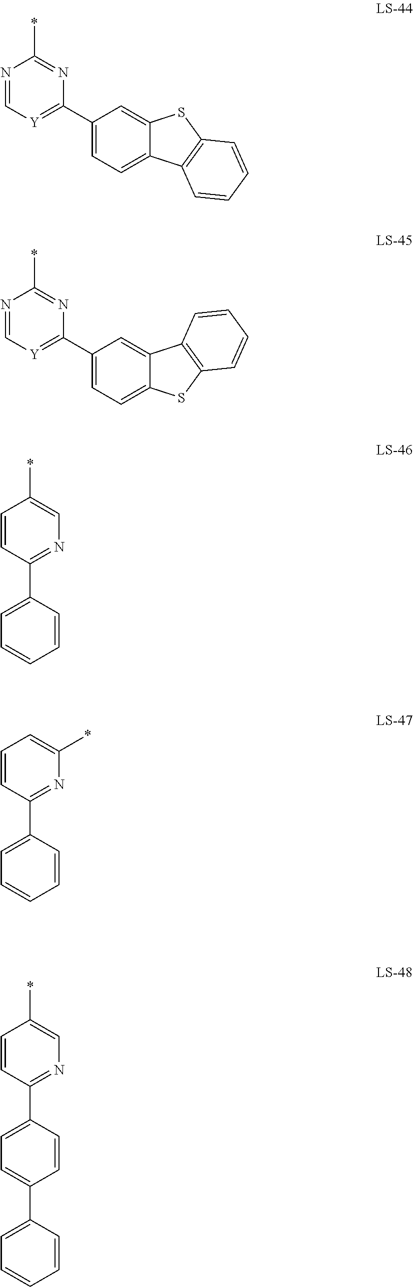

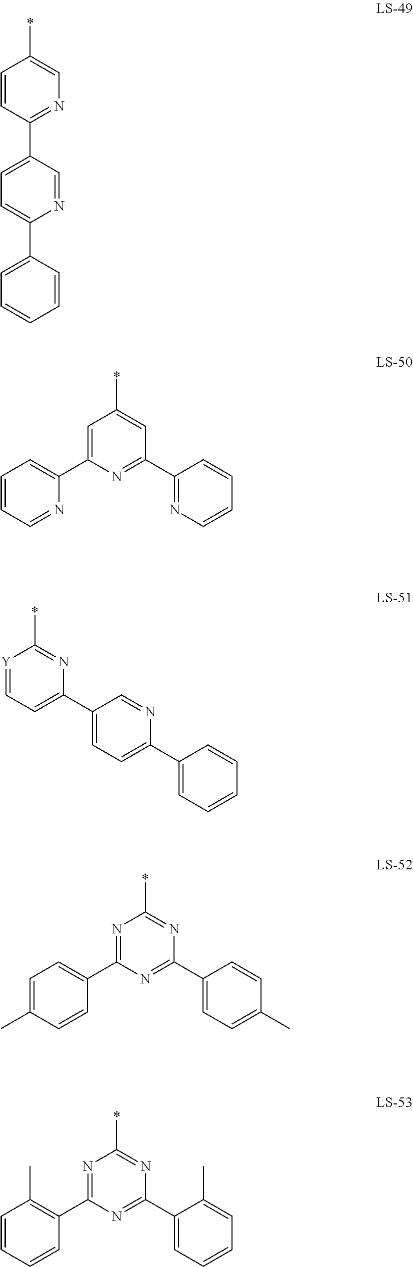

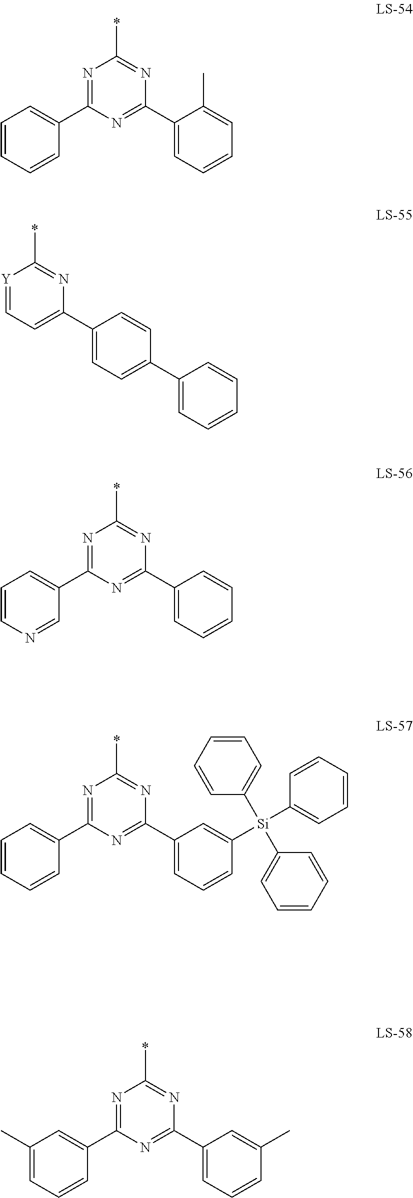

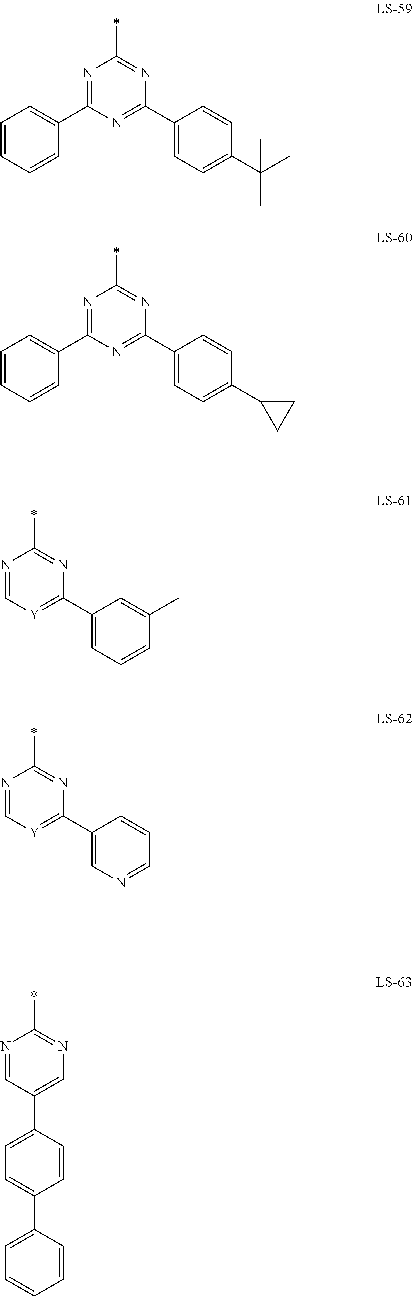

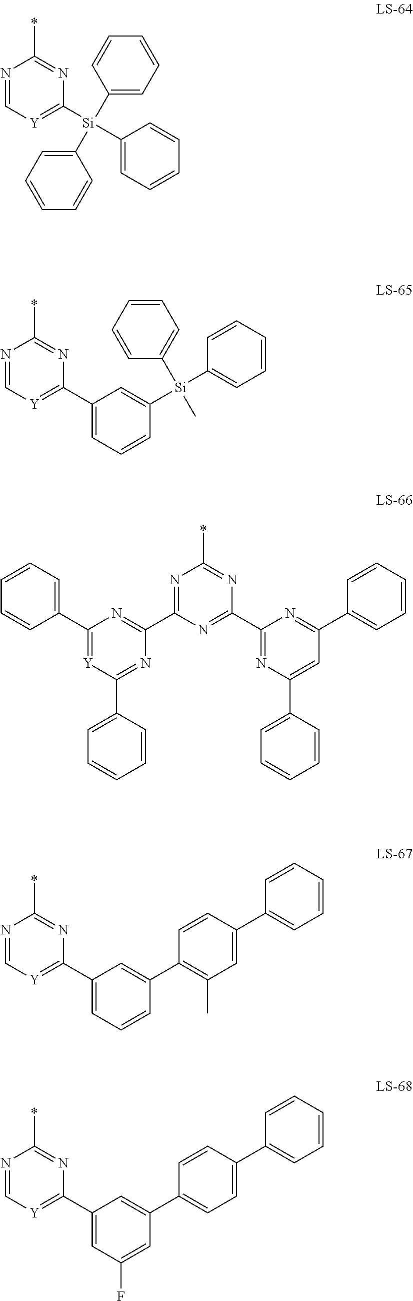

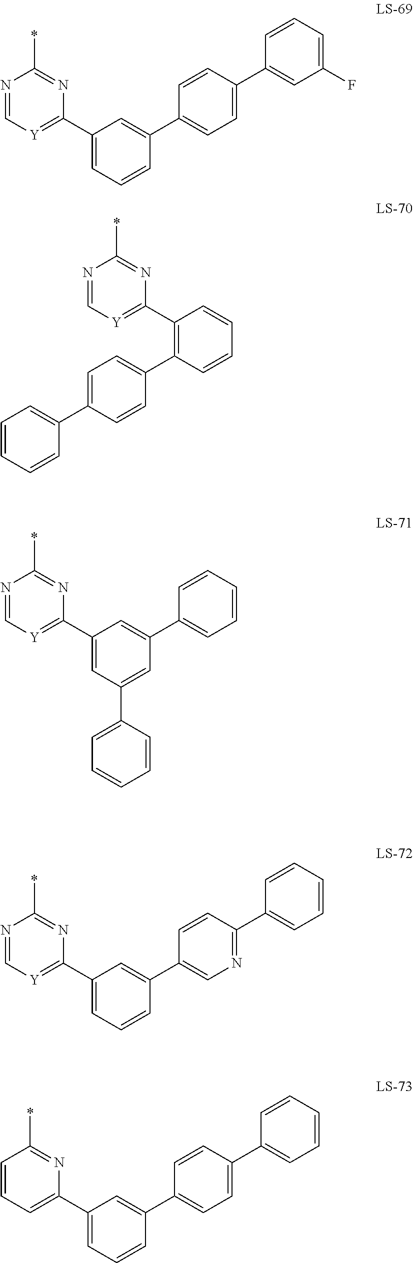

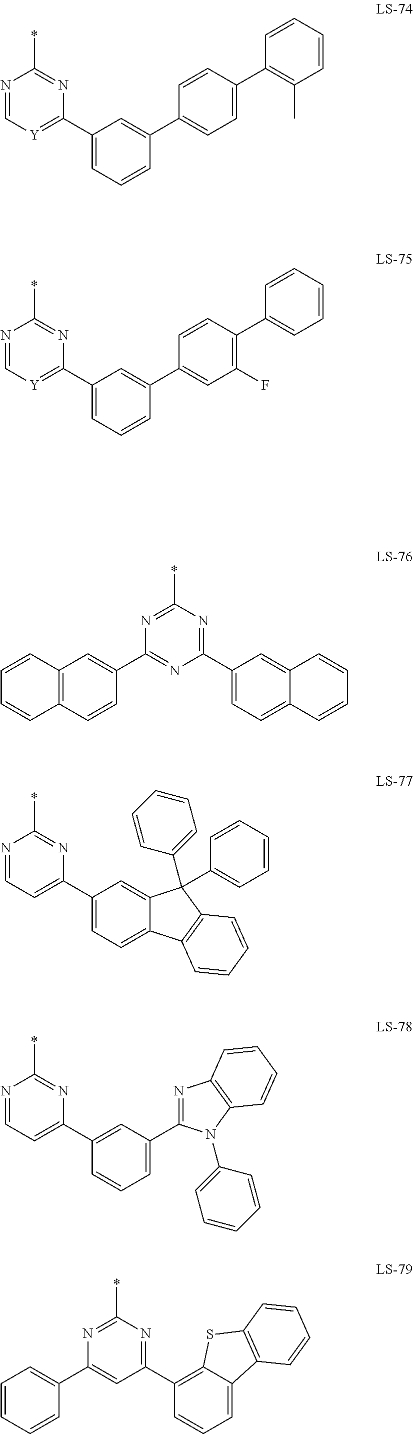

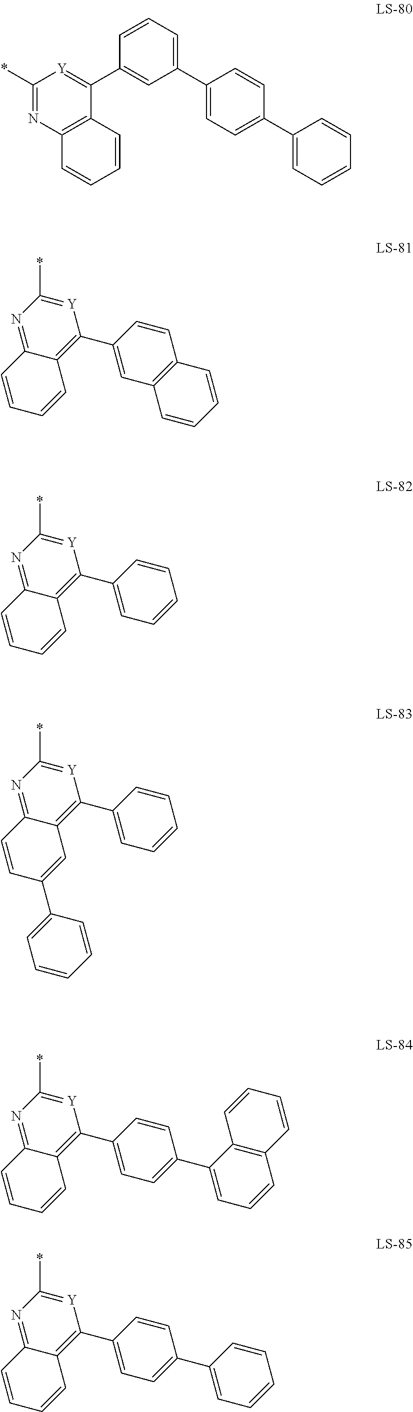

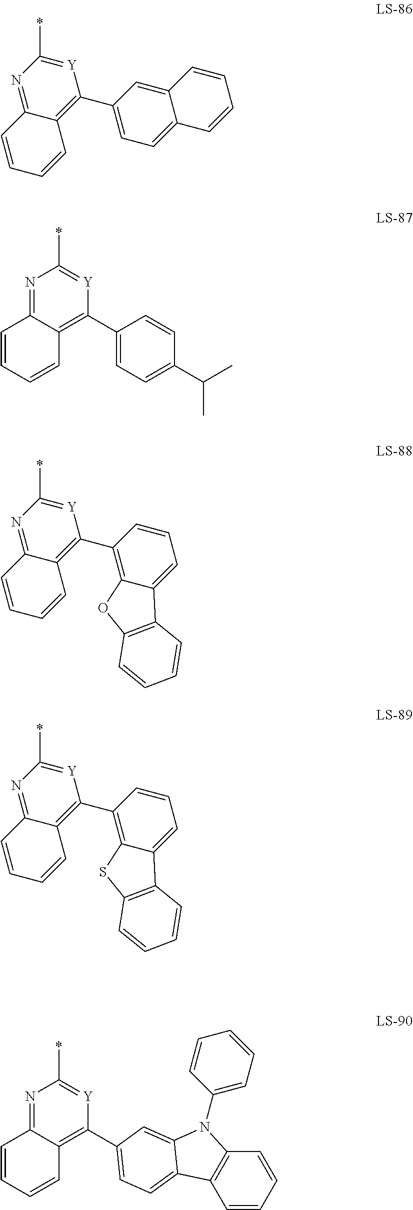

Herein, alkyl includes methyl, ethyl, n-propyl, isopropyl, n-butyl, isobutyl, tert-butyl, etc.; alkoxy includes methoxy, ethoxy, n-propoxy, isopropoxy, etc.; cycloalkyl includes cyclopropyl, cyclobutyl, cyclopentyl, cyclohexyl, etc.; and aryl includes phenyl, biphenyl, terphenyl, naphthyl, fluorenyl, phenanthrenyl, anthracenyl, indenyl, triphenylenyl, pyrenyl, tetracenyl, perylenyl, chrysenyl, naphthacenyl, fluoranthenyl, etc.

Herein, heteroaryl(ene) comprises at least one heteroatom selected from the group consisting of B, N, O, S, P(═O), Si and P; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Further, “halogen” includes F, Cl, Br and I.

Herein, “substituted” in the expression “substituted or unsubstituted” means that a hydrogen atom in a certain functional group is replaced with another atom or group, i.e., a substituent.

The substituents of the substituted alkyl, the substituted aryl(ene), the substituted heteroaryl(ene), the substituted cycloalkyl(ene), and the substituted alkoxy in the above formulae each independently are preferably at least one selected from the group consisting of deuterium, a halogen, a (C1-C30)alkyl unsubstituted or substituted with a halogen, a (C6-C30)aryl, a 3- to 30-membered heteroaryl unsubstituted or substituted with a (C6-C30)aryl, a 5- to 7-membered heterocycloalkyl, a 5- to 7-membered heterocycloalkyl fused with at least one (C6-C30)aromatic ring, a (C3-C30)cycloalkyl, a (C6-C30)cycloalkyl fused with at least one (C6-C30)aromatic ring, RₐR_{b}R_{c}Si—, a (C2-C30)alkenyl, a (C2-C30)alkynyl, a cyano, a carbazolyl, —NR_{d}Rₑ, —BR_{f}R_{g}, —PRₕRᵢ, —P(═O)RⱼRₖ, a (C6-C30)aryl(C1-C30)alkyl, a (C1-C30)alkyl(C6-C30)aryl, RₗW—, RₘC(═O)—, RₘC(═O)O—, a carboxyl, a nitro, and a hydroxyl, wherein Rₐ to Rₗ each independently represent a (C1-C30)alkyl, a (C6-C30)aryl, or a 3- to 30-membered heteroaryl, or are linked to an adjacent substituent(s) to form a mono- or polycyclic, 5- to 30-membered alicyclic or aromatic ring whose carbon atom(s) may be replaced with at least one hetero atom selected from the group consisting of nitrogen, oxygen and sulfur; W represents S or O; and Rₘ represents a (C1-C30)alkyl, a (C1-C30)alkoxy, a (C6-C30)aryl, or a (C6-C30)aryloxy.

The representative compounds of formula 2 include the following compounds, but are not limited thereto:

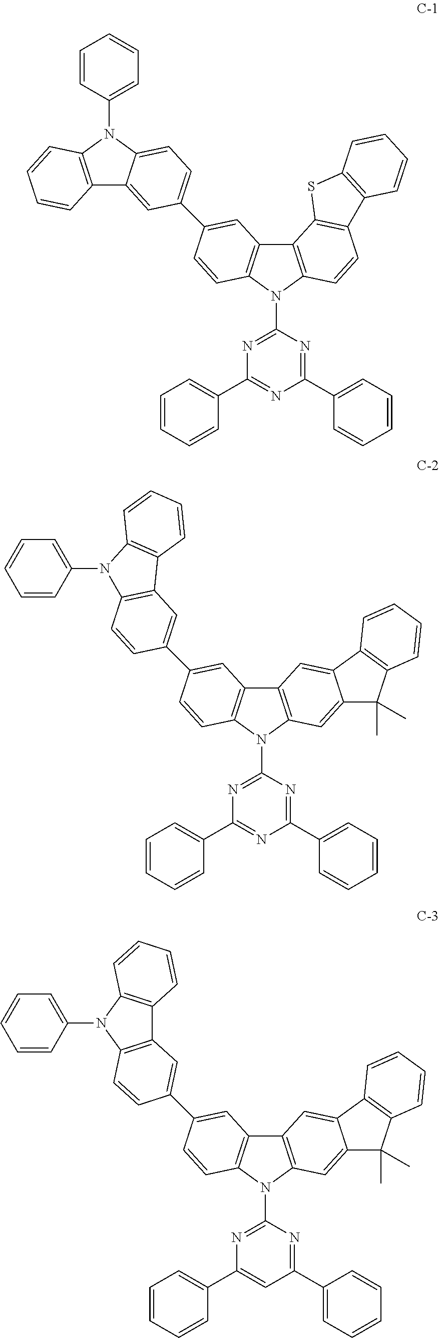 [SMILES: c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1 ; CC1(C)c2ccccc2-c2cc3c4cc(-c5ccc6c(c5)c5ccccc5n6-c5ccccc5)ccc4n(-c4nc(-c5ccccc5)nc(-c5ccccc5)n4)c3cc21 ; CC1(C)c2ccccc2-c2cc3c4cc(-c5ccc6c(c5)c5ccccc5n6-c5ccccc5)ccc4n(-c4nc(-c5ccccc5)cc(-c5ccccc5)n4)c3cc21]

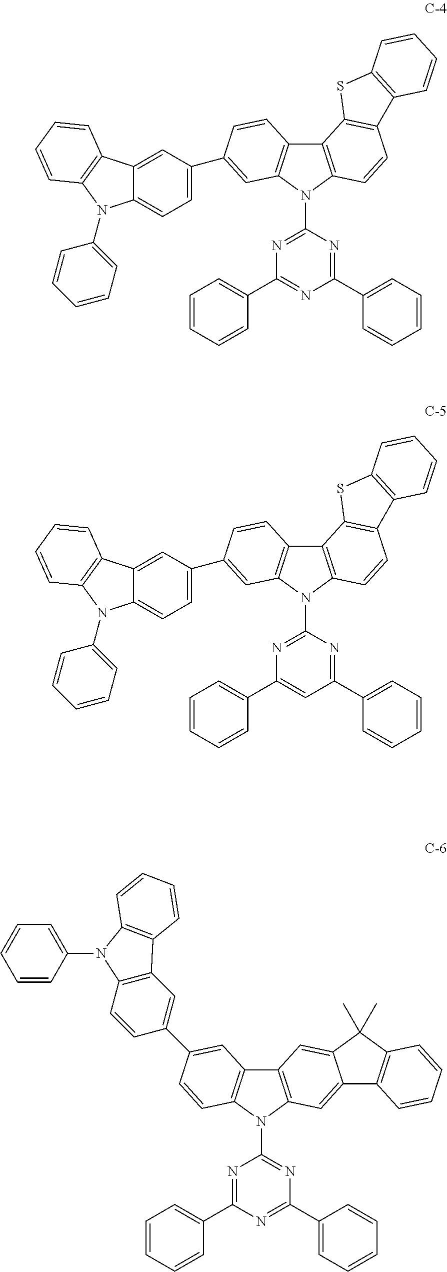 [SMILES: c1ccc(-c2nc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1 ; CC1(C)c2ccccc2-c2cc3c(cc21)c1cc(-c2ccc4c(c2)c2ccccc2n4-c2ccccc2)ccc1n3-c1nc(-c2ccccc2)nc(-c2ccccc2)n1]

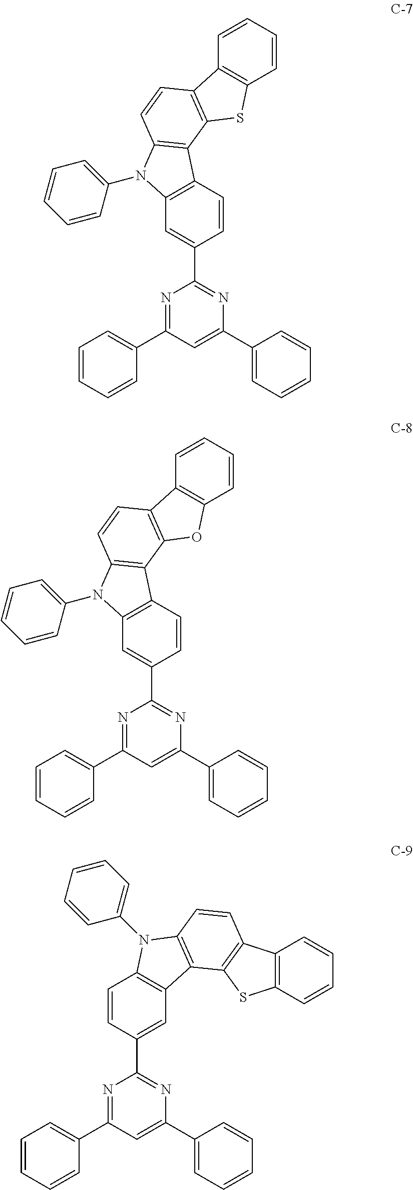 [SMILES: c1ccc(-c2cc(-c3ccccc3)nc(-c3ccc4c5c6sc7ccccc7c6ccc5n(-c5ccccc5)c4c3)n2)cc1 ; c1ccc(-c2cc(-c3ccccc3)nc(-c3ccc4c5c6oc7ccccc7c6ccc5n(-c5ccccc5)c4c3)n2)cc1 ; c1ccc(-c2cc(-c3ccccc3)nc(-c3ccc4c(c3)c3c5sc6ccccc6c5ccc3n4-c3ccccc3)n2)cc1]

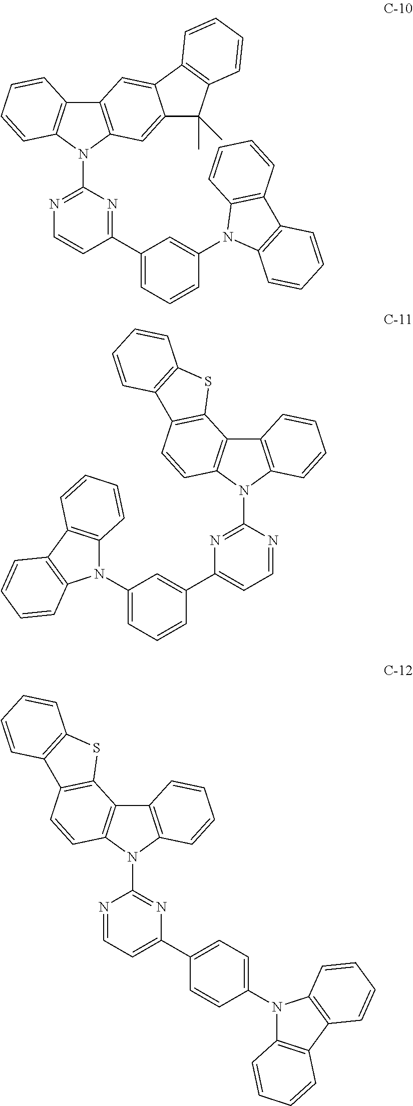

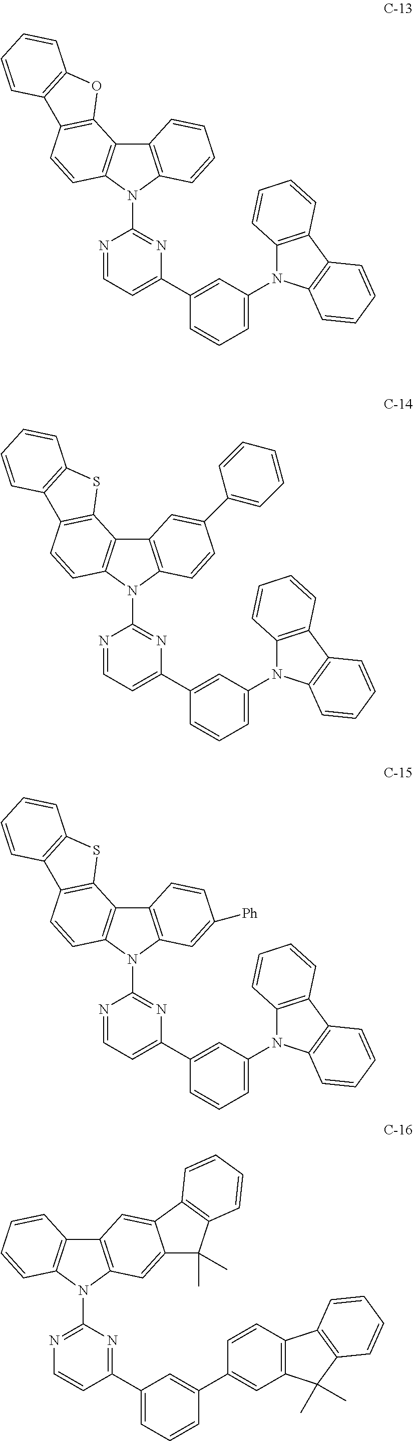

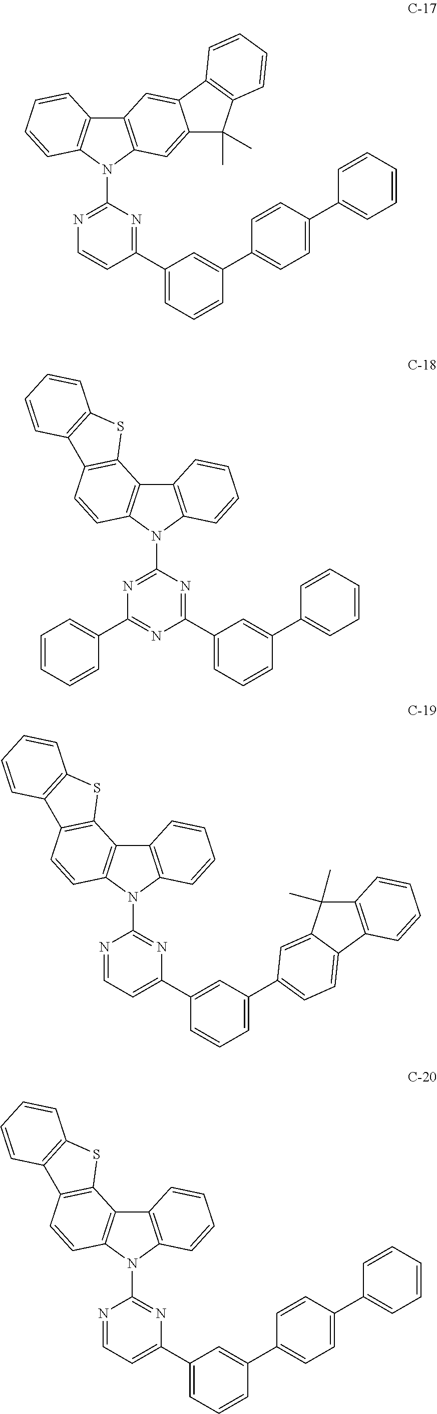 [SMILES: CC1(C)c2ccccc2-c2cc3c4ccccc4n(-c4nccc(-c5cccc(-c6ccc(-c7ccccc7)cc6)c5)n4)c3cc21 ; c1ccc(-c2cccc(-c3nc(-c4ccccc4)nc(-n4c5ccccc5c5c6sc7ccccc7c6ccc54)n3)c2)cc1 ; CC1(C)c2ccccc2-c2ccc(-c3cccc(-c4ccnc(-n5c6ccccc6c6c7sc8ccccc8c7ccc65)n4)c3)cc21 ; c1ccc(-c2ccc(-c3cccc(-c4ccnc(-n5c6ccccc6c6c7sc8ccccc8c7ccc65)n4)c3)cc2)cc1]

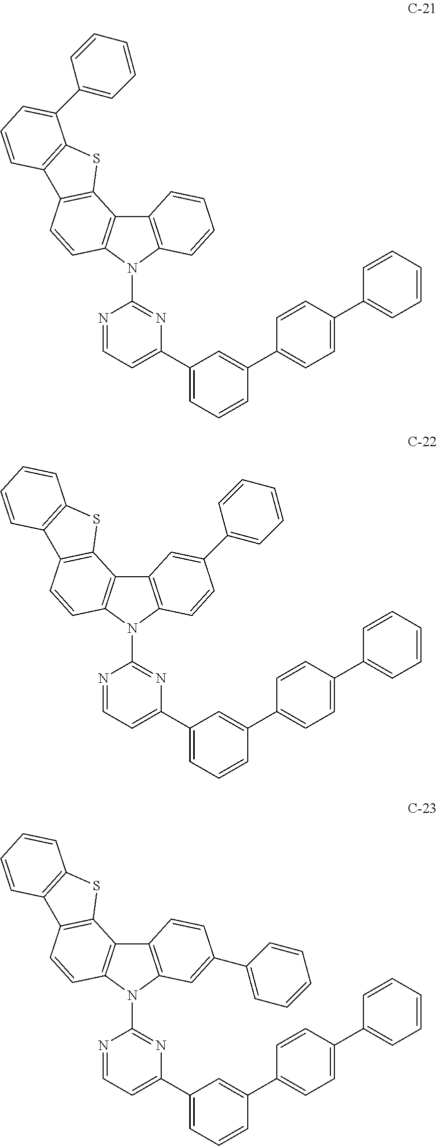 [SMILES: c1ccc(-c2ccc(-c3cccc(-c4ccnc(-n5c6ccccc6c6c7sc8c(-c9ccccc9)cccc8c7ccc65)n4)c3)cc2)cc1 ; c1ccc(-c2ccc(-c3cccc(-c4ccnc(-n5c6ccc(-c7ccccc7)cc6c6c7sc8ccccc8c7ccc65)n4)c3)cc2)cc1 ; c1ccc(-c2ccc(-c3cccc(-c4ccnc(-n5c6cc(-c7ccccc7)ccc6c6c7sc8ccccc8c7ccc65)n4)c3)cc2)cc1]

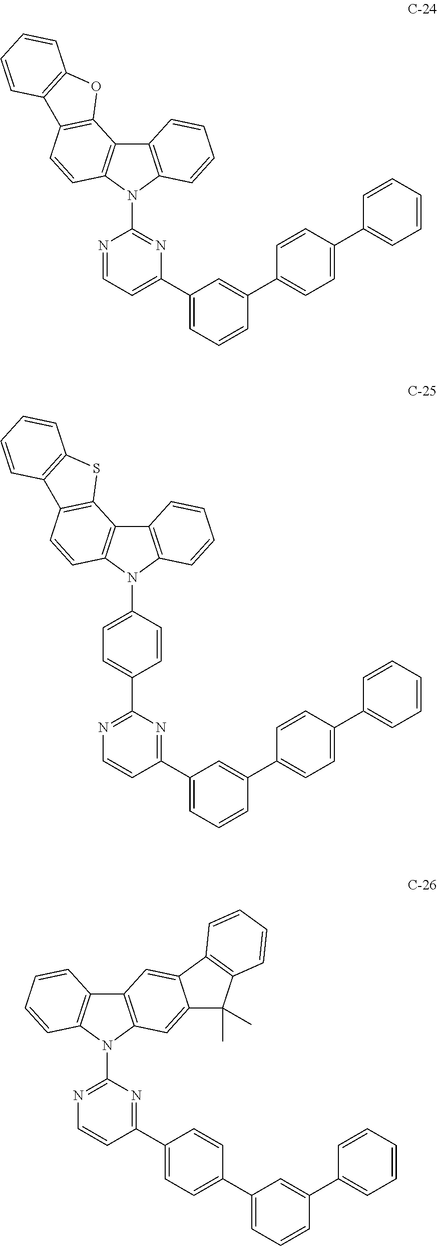 [SMILES: c1ccc(-c2ccc(-c3cccc(-c4ccnc(-n5c6ccccc6c6c7oc8ccccc8c7ccc65)n4)c3)cc2)cc1 ; c1ccc(-c2ccc(-c3cccc(-c4ccnc(-c5ccc(-n6c7ccccc7c7c8sc9ccccc9c8ccc76)cc5)n4)c3)cc2)cc1 ; CC1(C)c2ccccc2-c2cc3c4ccccc4n(-c4nccc(-c5ccc(-c6cccc(-c7ccccc7)c6)cc5)n4)c3cc21]

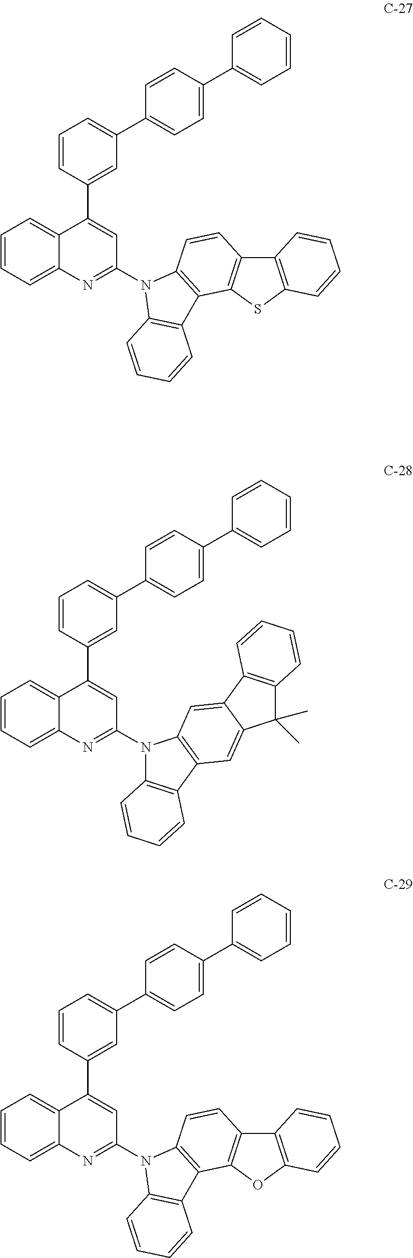 [SMILES: c1ccc(-c2ccc(-c3cccc(-c4cc(-n5c6ccccc6c6c7sc8ccccc8c7ccc65)nc5ccccc45)c3)cc2)cc1 ; CC1(C)c2ccccc2-c2cc3c(cc21)c1ccccc1n3-c1cc(-c2cccc(-c3ccc(-c4ccccc4)cc3)c2)c2ccccc2n1 ; c1ccc(-c2ccc(-c3cccc(-c4cc(-n5c6ccccc6c6c7oc8ccccc8c7ccc65)nc5ccccc45)c3)cc2)cc1]

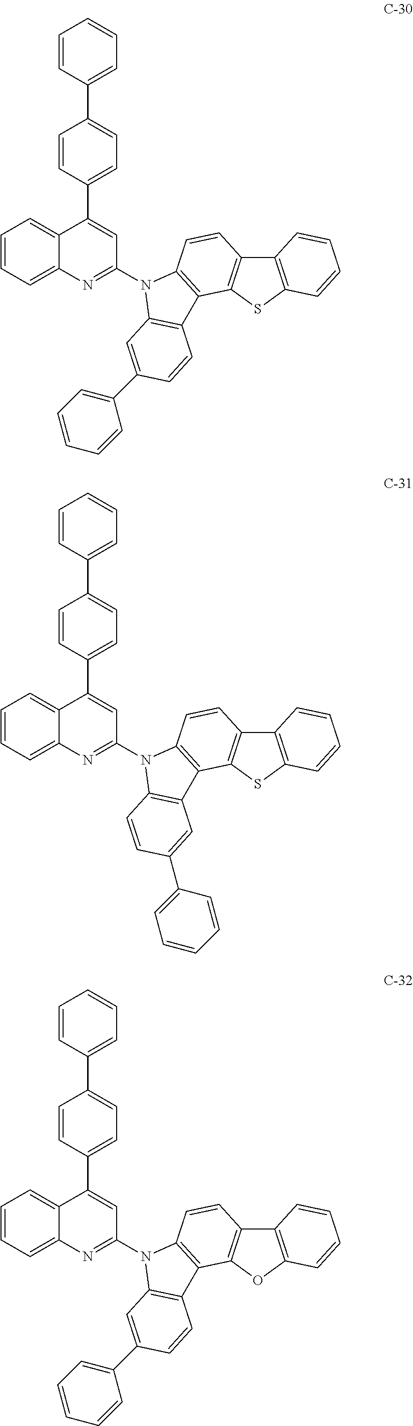

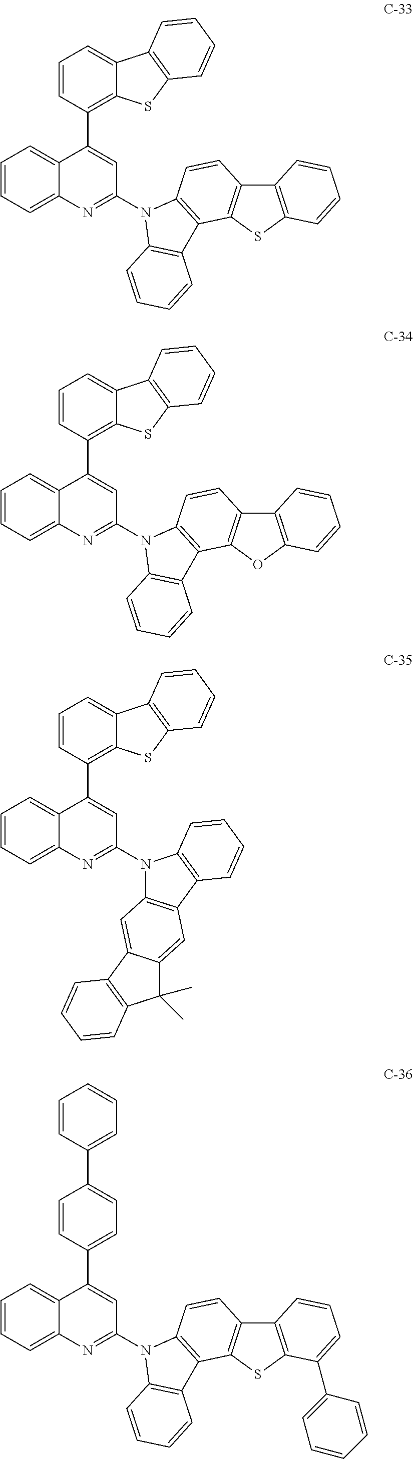

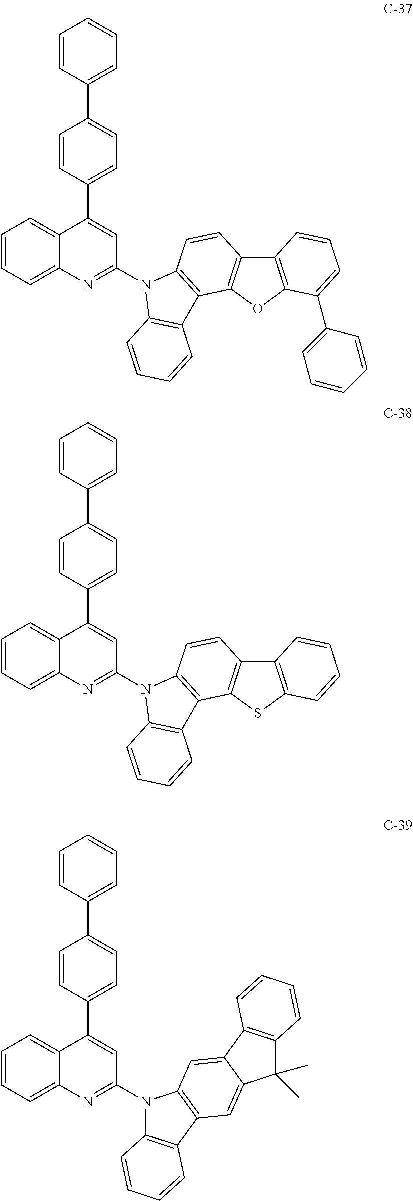 [SMILES: c1ccc(-c2ccc(-c3cc(-n4c5ccccc5c5c6oc7c(-c8ccccc8)cccc7c6ccc54)nc4ccccc34)cc2)cc1 ; c1ccc(-c2ccc(-c3cc(-n4c5ccccc5c5c6sc7ccccc7c6ccc54)nc4ccccc34)cc2)cc1 ; CC1(C)c2ccccc2-c2cc3c(cc21)c1ccccc1n3-c1cc(-c2ccc(-c3ccccc3)cc2)c2ccccc2n1]

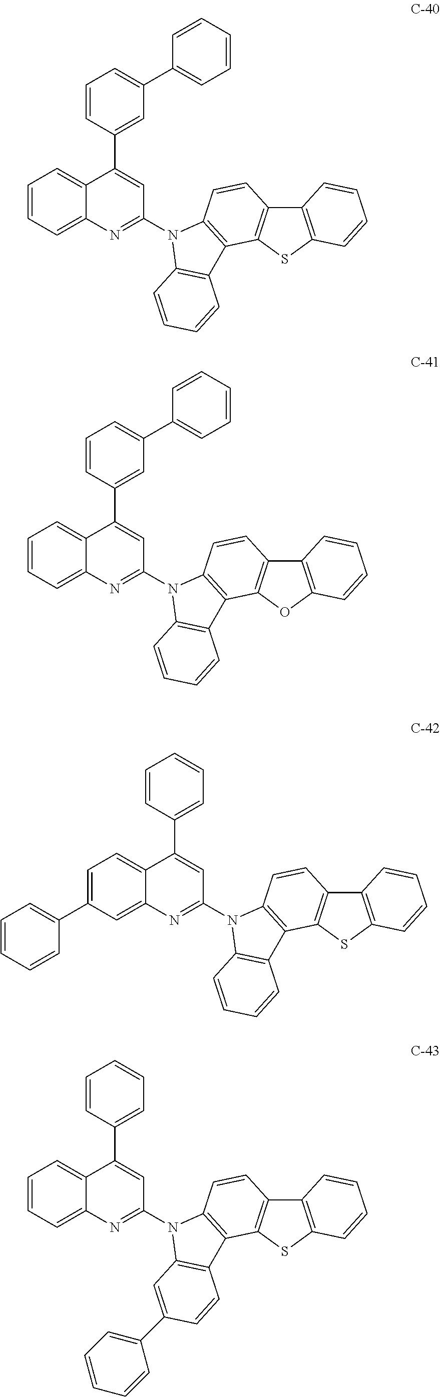

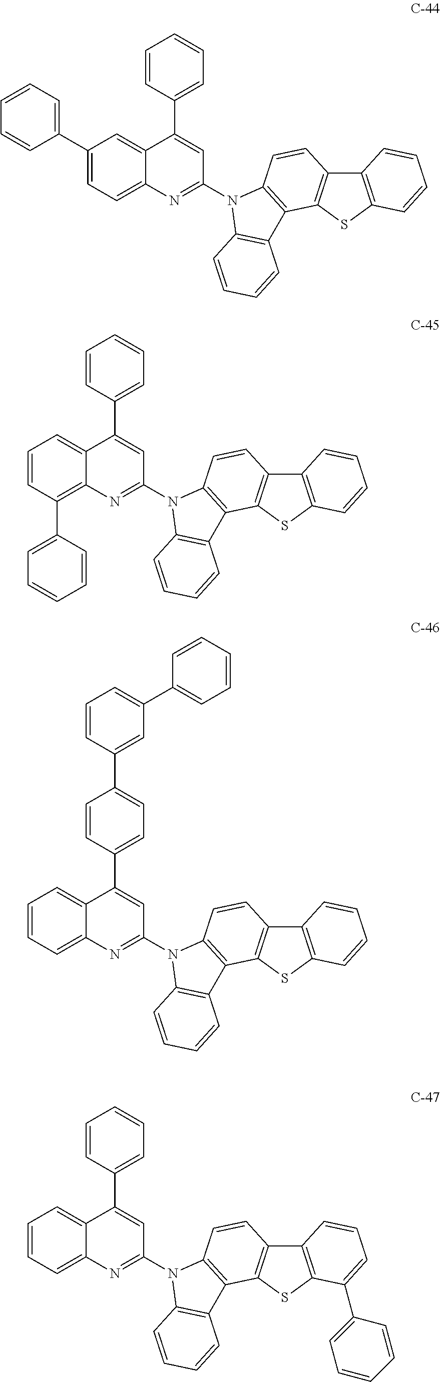

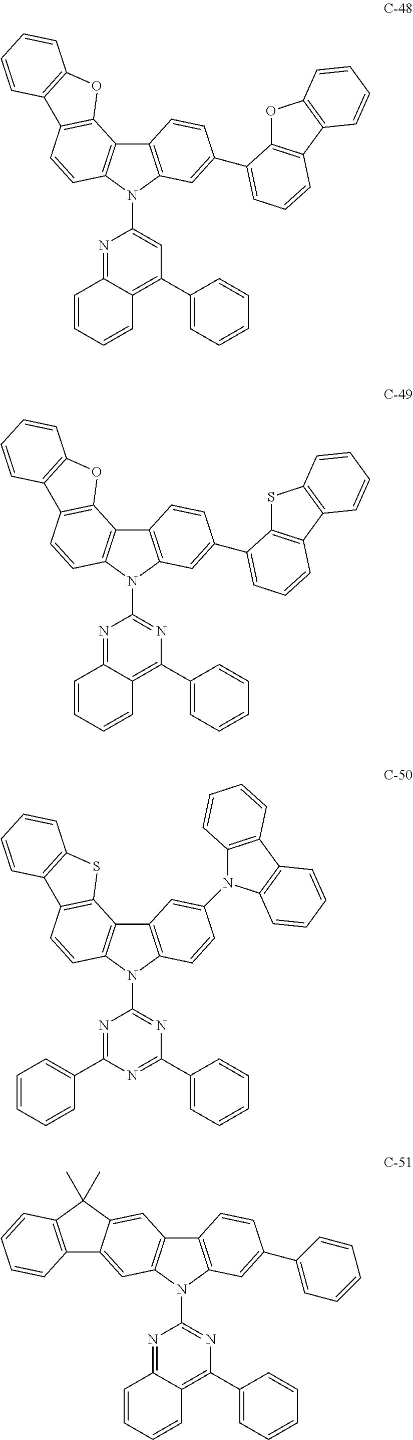 [SMILES: c1ccc(-c2cc(-n3c4cc(-c5cccc6c5oc5ccccc56)ccc4c4c5oc6ccccc6c5ccc43)nc3ccccc23)cc1 ; c1ccc(-c2nc(-n3c4cc(-c5cccc6c5sc5ccccc56)ccc4c4c5oc6ccccc6c5ccc43)nc3ccccc23)cc1 ; c1ccc(-c2nc(-c3ccccc3)nc(-n3c4ccc(-n5c6ccccc6c6ccccc65)cc4c4c5sc6ccccc6c5ccc43)n2)cc1 ; CC1(C)c2ccccc2-c2cc3c(cc21)c1ccc(-c2ccccc2)cc1n3-c1nc(-c2ccccc2)c2ccccc2n1]

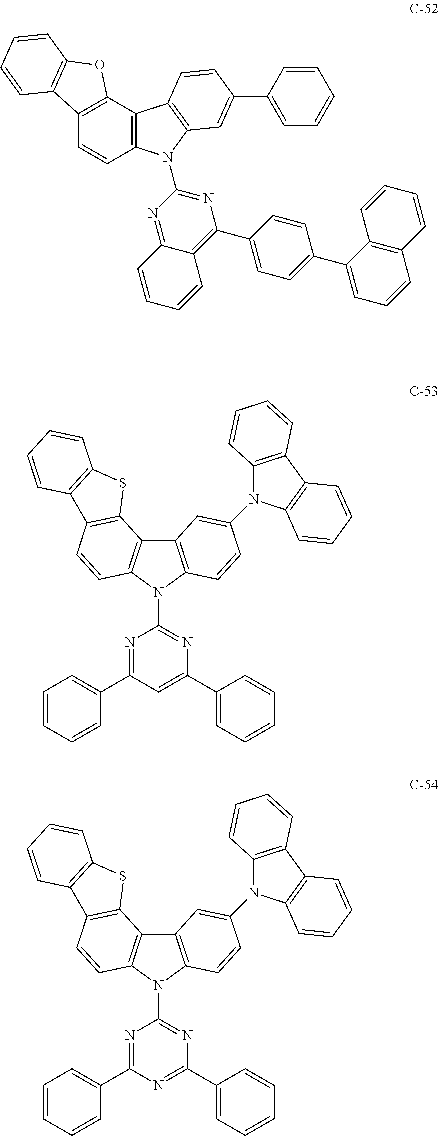 [SMILES: c1ccc(-c2ccc3c4c5oc6ccccc6c5ccc4n(-c4nc(-c5ccc(-c6cccc7ccccc67)cc5)c5ccccc5n4)c3c2)cc1 ; c1ccc(-c2cc(-c3ccccc3)nc(-n3c4ccc(-n5c6ccccc6c6ccccc65)cc4c4c5sc6ccccc6c5ccc43)n2)cc1 ; c1ccc(-c2nc(-c3ccccc3)nc(-n3c4ccc(-n5c6ccccc6c6ccccc65)cc4c4c5sc6ccccc6c5ccc43)n2)cc1]

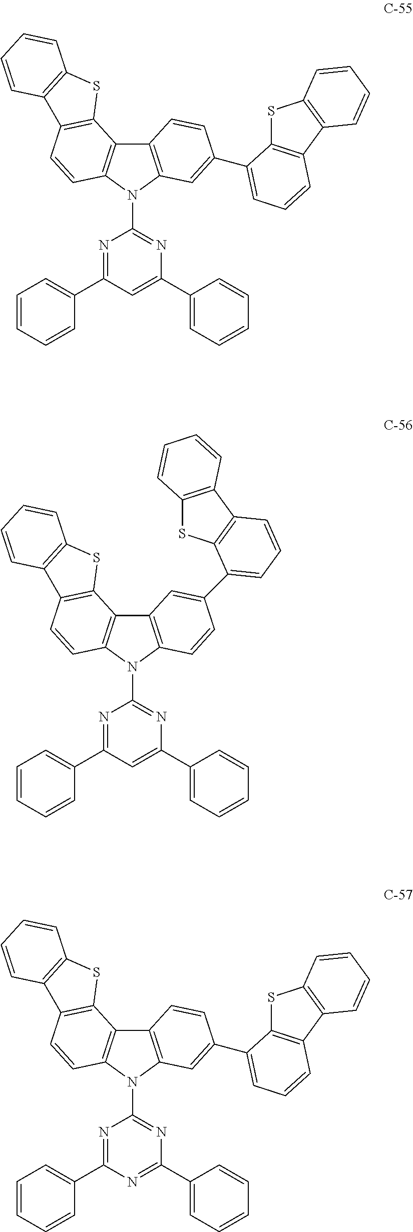 [SMILES: c1ccc(-c2cc(-c3ccccc3)nc(-n3c4cc(-c5cccc6c5sc5ccccc56)ccc4c4c5sc6ccccc6c5ccc43)n2)cc1 ; c1ccc(-c2cc(-c3ccccc3)nc(-n3c4ccc(-c5cccc6c5sc5ccccc56)cc4c4c5sc6ccccc6c5ccc43)n2)cc1 ; c1ccc(-c2nc(-c3ccccc3)nc(-n3c4cc(-c5cccc6c5sc5ccccc56)ccc4c4c5sc6ccccc6c5ccc43)n2)cc1]

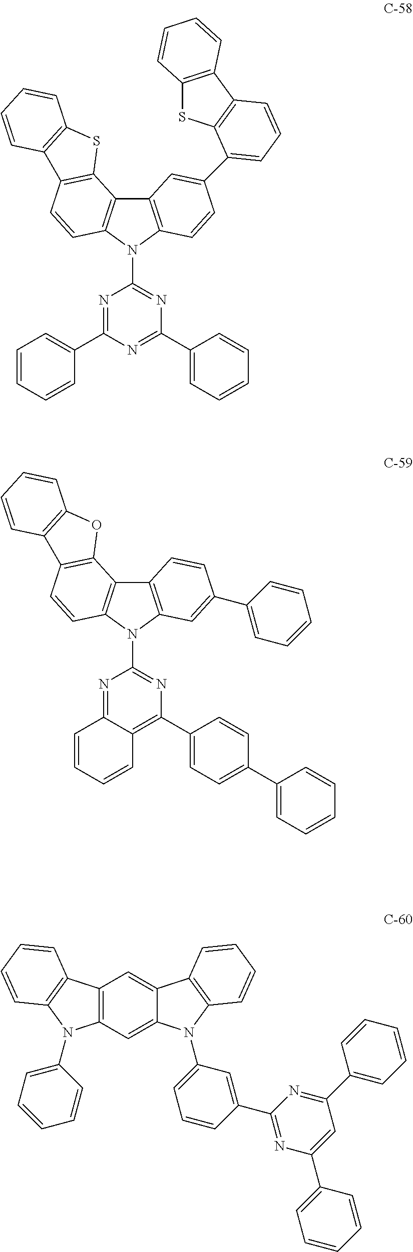 [SMILES: c1ccc(-c2nc(-c3ccccc3)nc(-n3c4ccc(-c5cccc6c5sc5ccccc56)cc4c4c5sc6ccccc6c5ccc43)n2)cc1 ; c1ccc(-c2ccc(-c3nc(-n4c5cc(-c6ccccc6)ccc5c5c6oc7ccccc7c6ccc54)nc4ccccc34)cc2)cc1 ; c1ccc(-c2cc(-c3ccccc3)nc(-c3cccc(-n4c5ccccc5c5cc6c7ccccc7n(-c7ccccc7)c6cc54)c3)n2)cc1]

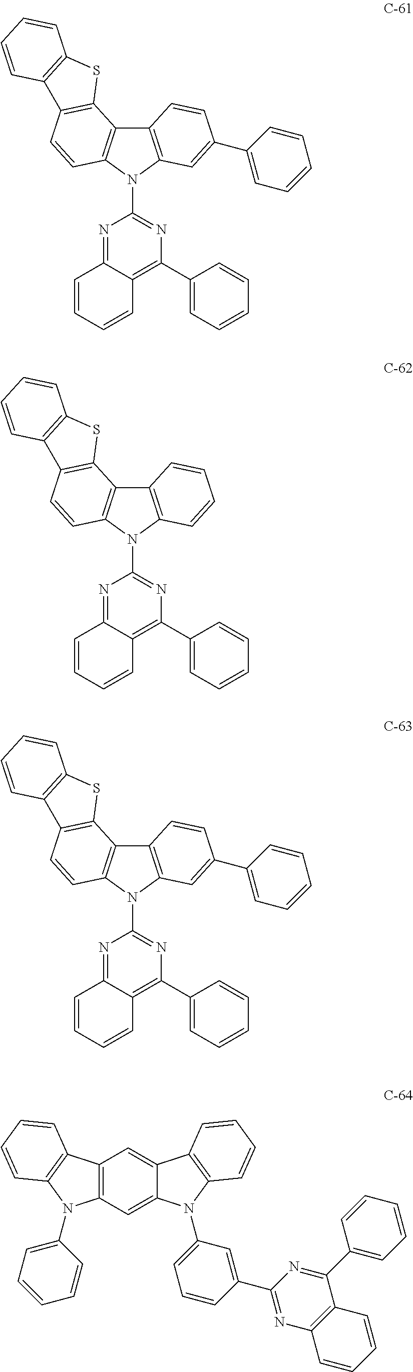 [SMILES: c1ccc(-c2ccc3c4c5sc6ccccc6c5ccc4n(-c4nc(-c5ccccc5)c5ccccc5n4)c3c2)cc1 ; c1ccc(-c2nc(-n3c4ccccc4c4c5sc6ccccc6c5ccc43)nc3ccccc23)cc1 ; c1ccc(-c2ccc3c4c5sc6ccccc6c5ccc4n(-c4nc(-c5ccccc5)c5ccccc5n4)c3c2)cc1 ; c1ccc(-c2nc(-c3cccc(-n4c5ccccc5c5cc6c7ccccc7n(-c7ccccc7)c6cc54)c3)nc3ccccc23)cc1]

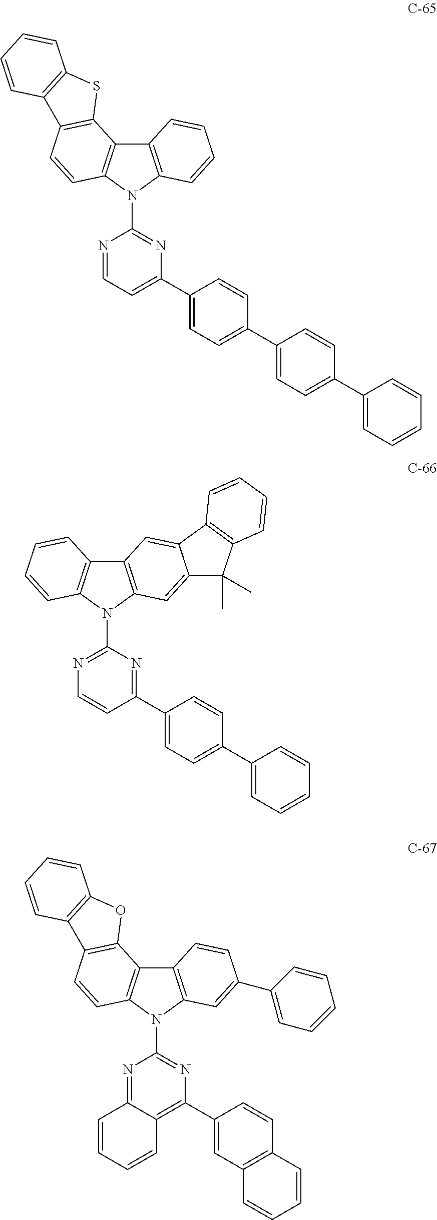 [SMILES: c1ccc(-c2ccc(-c3ccc(-c4ccnc(-n5c6ccccc6c6c7sc8ccccc8c7ccc65)n4)cc3)cc2)cc1 ; CC1(C)c2ccccc2-c2cc3c4ccccc4n(-c4nccc(-c5ccc(-c6ccccc6)cc5)n4)c3cc21 ; c1ccc(-c2ccc3c4c5oc6ccccc6c5ccc4n(-c4nc(-c5ccc6ccccc6c5)c5ccccc5n4)c3c2)cc1]

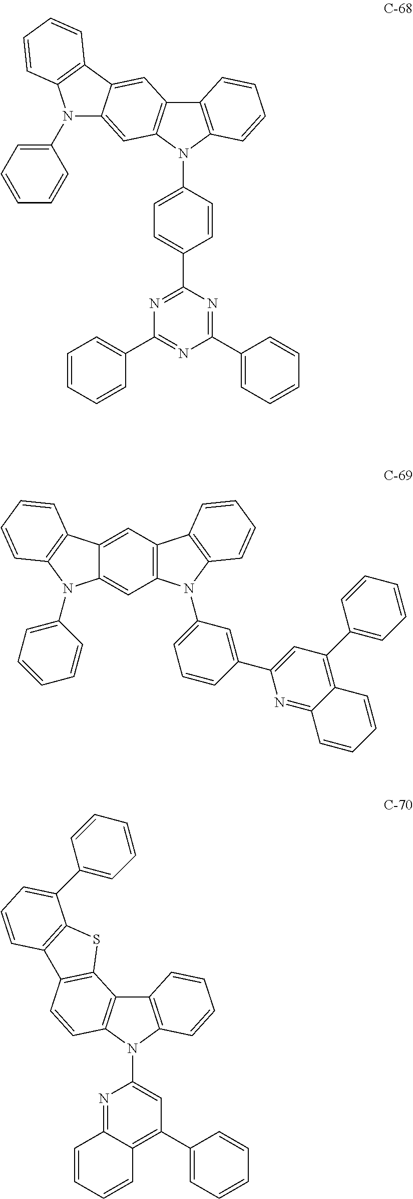 [SMILES: c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2cc(-c3cccc(-n4c5ccccc5c5cc6c7ccccc7n(-c7ccccc7)c6cc54)c3)nc3ccccc23)cc1 ; c1ccc(-c2cc(-n3c4ccccc4c4c5sc6c(-c7ccccc7)cccc6c5ccc43)nc3ccccc23)cc1]

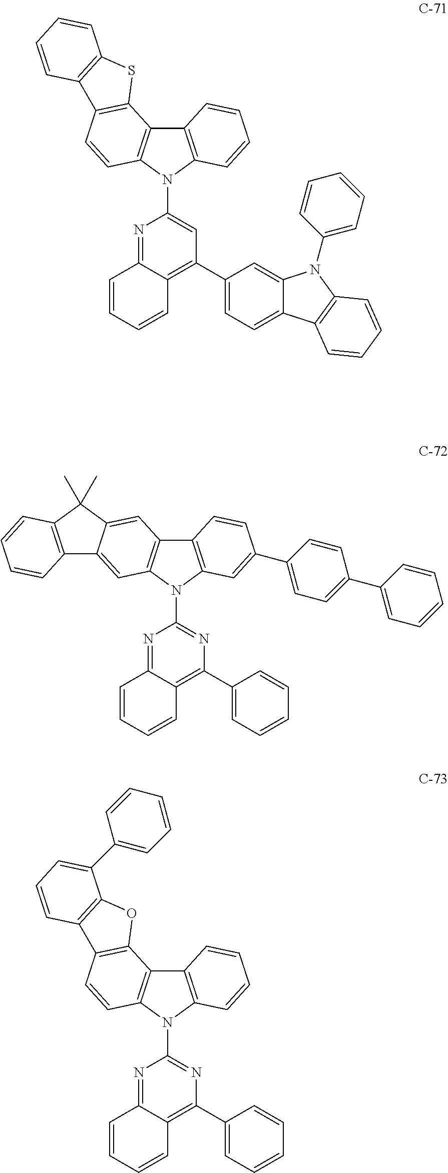 [SMILES: c1ccc(-n2c3ccccc3c3ccc(-c4cc(-n5c6ccccc6c6c7sc8ccccc8c7ccc65)nc5ccccc45)cc32)cc1 ; CC1(C)c2ccccc2-c2cc3c(cc21)c1ccc(-c2ccc(-c4ccccc4)cc2)cc1n3-c1nc(-c2ccccc2)c2ccccc2n1 ; c1ccc(-c2nc(-n3c4ccccc4c4c5oc6c(-c7ccccc7)cccc6c5ccc43)nc3ccccc23)cc1]

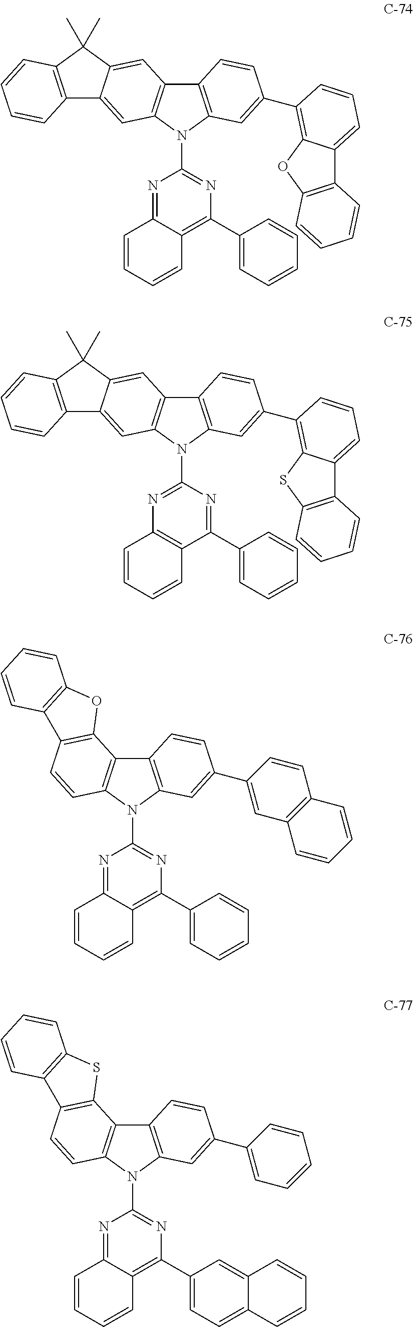

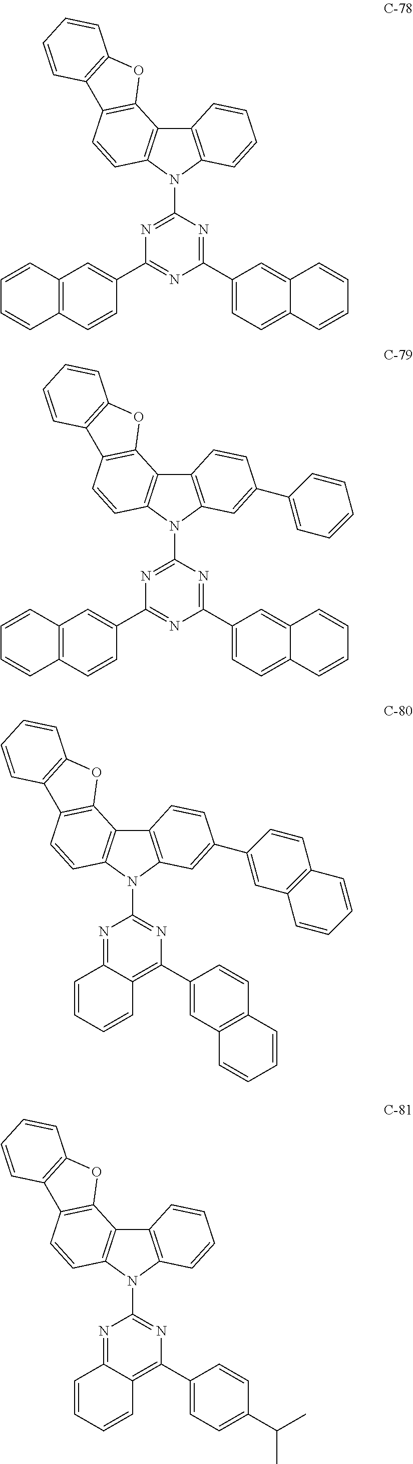

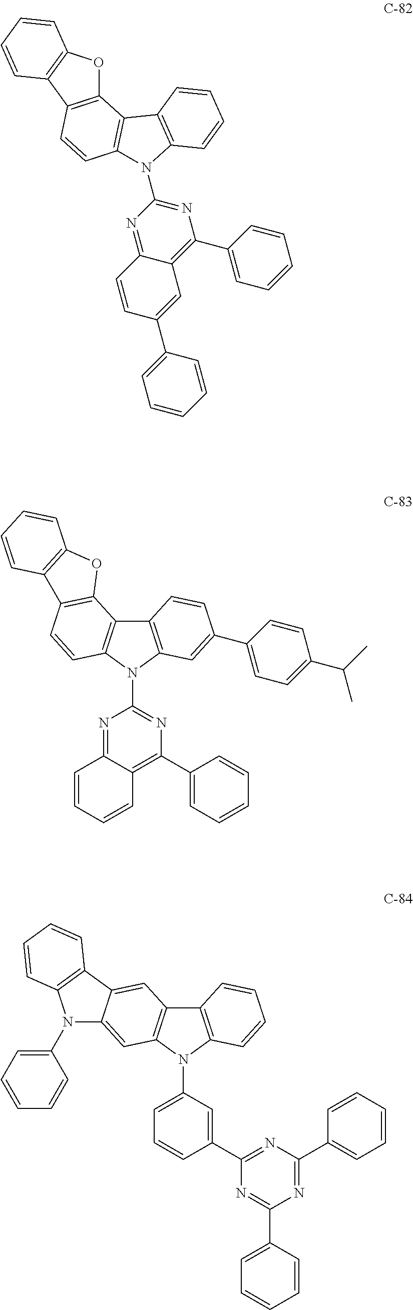 [SMILES: c1ccc(-c2ccc3nc(-n4c5ccccc5c5c6oc7ccccc7c6ccc54)nc(-c4ccccc4)c3c2)cc1 ; CC(C)c1ccc(-c2ccc3c4c5oc6ccccc6c5ccc4n(-c4nc(-c5ccccc5)c5ccccc5n4)c3c2)cc1 ; c1ccc(-c2nc(-c3ccccc3)nc(-c3cccc(-n4c5ccccc5c5cc6c7ccccc7n(-c7ccccc7)c6cc54)c3)n2)cc1]

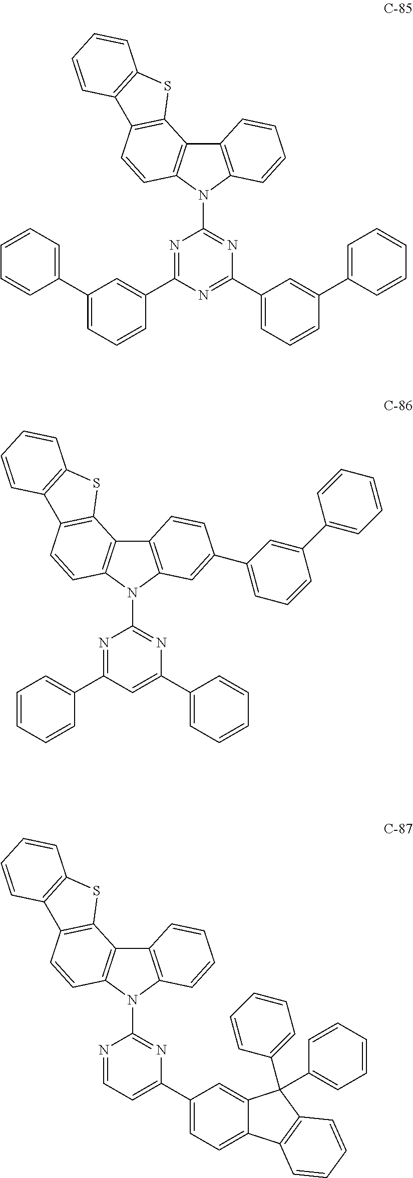 [SMILES: c1ccc(-c2cccc(-c3nc(-c4cccc(-c5ccccc5)c4)nc(-n4c5ccccc5c5c6sc7ccccc7c6ccc54)n3)c2)cc1 ; c1ccc(-c2cccc(-c3ccc4c5c6sc7ccccc7c6ccc5n(-c5nc(-c6ccccc6)cc(-c6ccccc6)n5)c4c3)c2)cc1 ; c1ccc(C2(c3ccccc3)c3ccccc3-c3ccc(-c4ccnc(-n5c6ccccc6c6c7sc8ccccc8c7ccc65)n4)cc32)cc1]

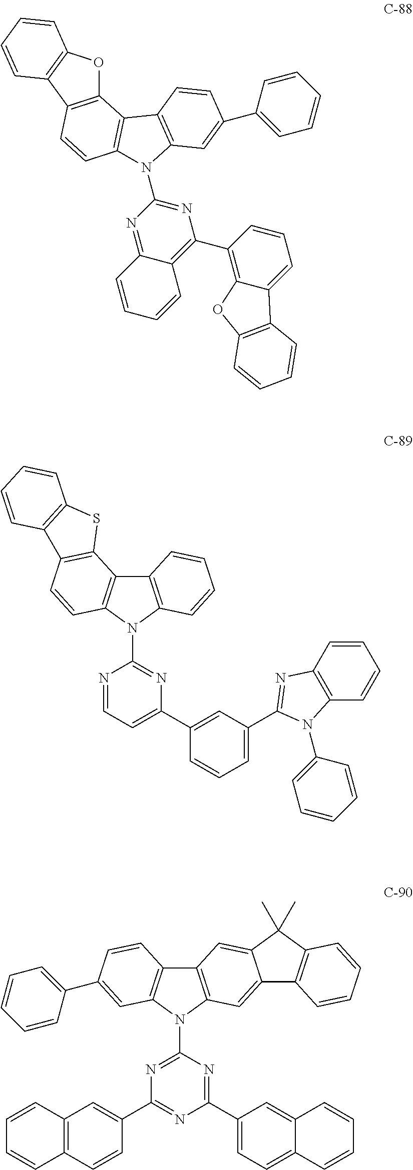 [SMILES: c1ccc(-c2ccc3c4c5oc6ccccc6c5ccc4n(-c4nc(-c5cccc6c5oc5ccccc56)c5ccccc5n4)c3c2)cc1 ; c1ccc(-n2c(-c3cccc(-c4ccnc(-n5c6ccccc6c6c7sc8ccccc8c7ccc65)n4)c3)nc3ccccc32)cc1 ; CC1(C)c2ccccc2-c2cc3c(cc21)c1ccc(-c2ccccc2)cc1n3-c1nc(-c2ccc3ccccc3c2)nc(-c2ccc3ccccc3c2)n1]

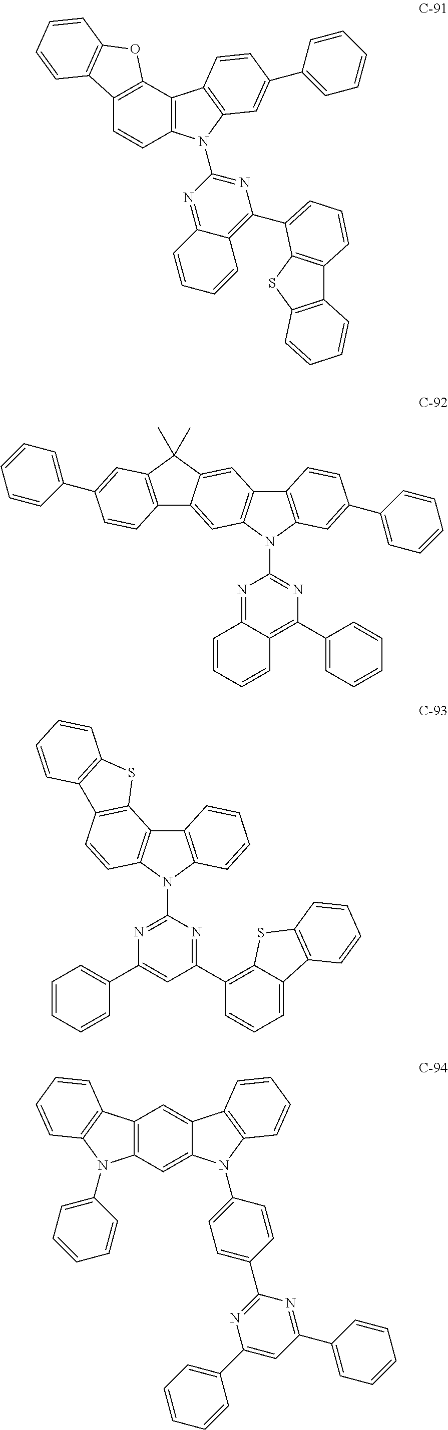 [SMILES: c1ccc(-c2ccc3c4c5oc6ccccc6c5ccc4n(-c4nc(-c5cccc6c5sc5ccccc56)c5ccccc5n4)c3c2)cc1 ; CC1(C)c2cc(-c3ccccc3)ccc2-c2cc3c(cc21)c1ccc(-c2ccccc2)cc1n3-c1nc(-c2ccccc2)c2ccccc2n1 ; c1ccc(-c2cc(-c3cccc4c3sc3ccccc34)nc(-n3c4ccccc4c4c5sc6ccccc6c5ccc43)n2)cc1 ; c1ccc(-c2cc(-c3ccccc3)nc(-c3ccc(-n4c5ccccc5c5cc6c7ccccc7n(-c7ccccc7)c6cc54)cc3)n2)cc1]

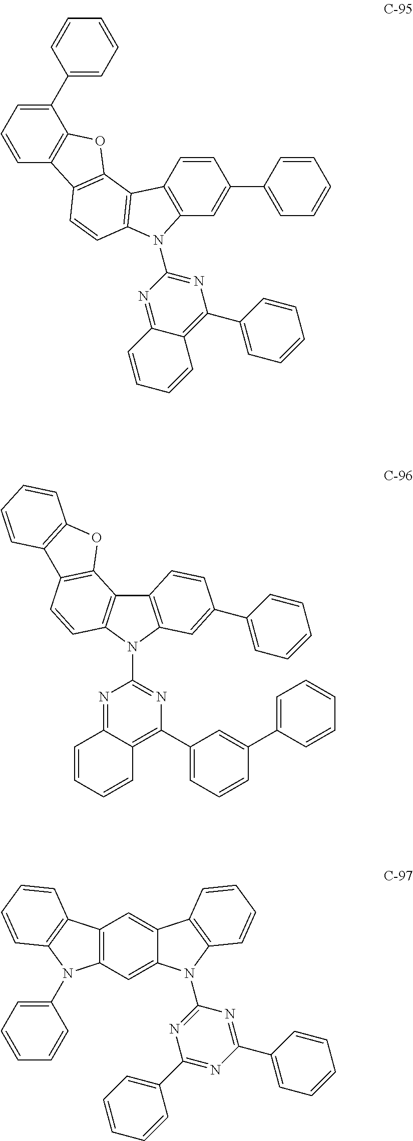 [SMILES: c1ccc(-c2ccc3c4c5oc6c(-c7ccccc7)cccc6c5ccc4n(-c4nc(-c5ccccc5)c5ccccc5n4)c3c2)cc1 ; c1ccc(-c2cccc(-c3nc(-n4c5cc(-c6ccccc6)ccc5c5c6oc7ccccc7c6ccc54)nc4ccccc34)c2)cc1 ; c1ccc(-c2nc(-c3ccccc3)nc(-n3c4ccccc4c4cc5c6ccccc6n(-c6ccccc6)c5cc43)n2)cc1]

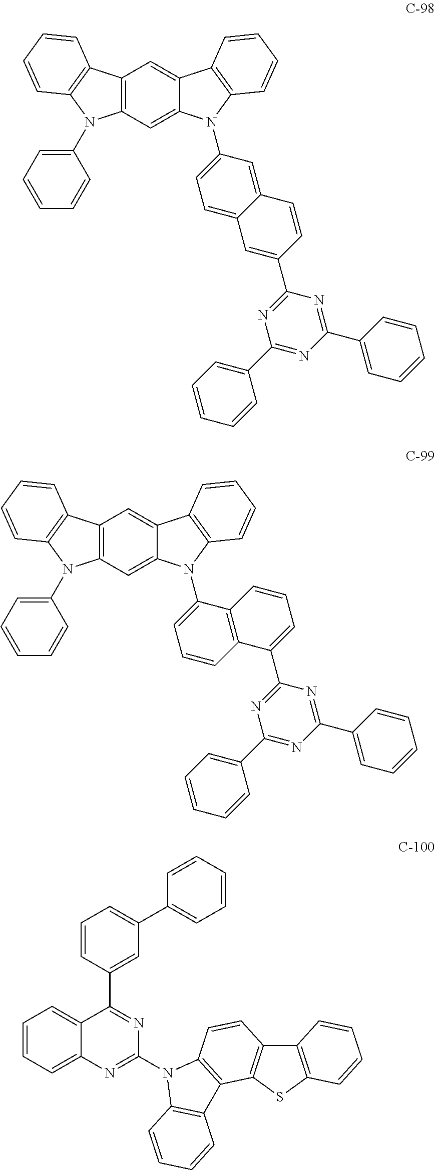 [SMILES: c1ccc(-c2nc(-c3ccccc3)nc(-c3ccc4cc(-n5c6ccccc6c6cc7c8ccccc8n(-c8ccccc8)c7cc65)ccc4c3)n2)cc1 ; c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2cccc(-c3nc(-n4c5ccccc5c5c6sc7ccccc7c6ccc54)nc4ccccc34)c2)cc1]

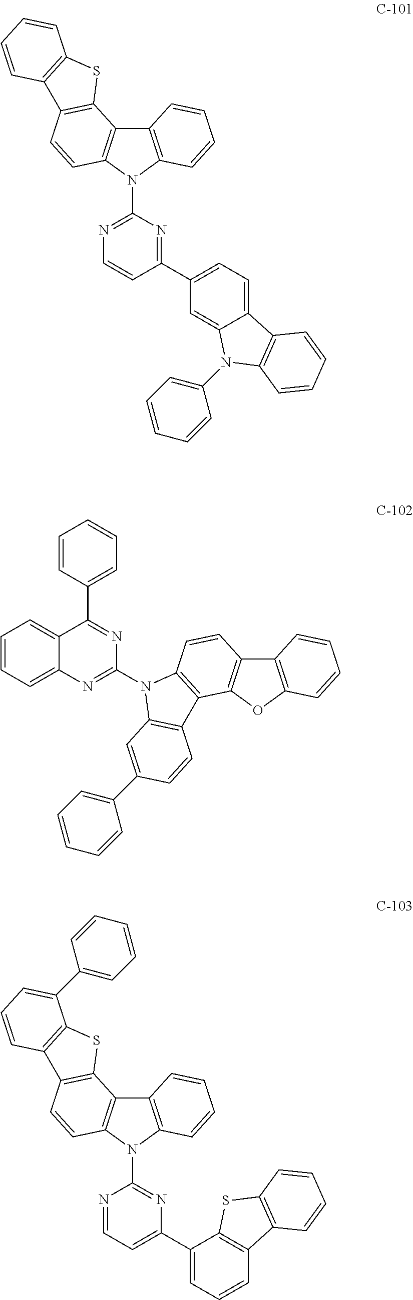 [SMILES: c1ccc(-n2c3ccccc3c3ccc(-c4ccnc(-n5c6ccccc6c6c7sc8ccccc8c7ccc65)n4)cc32)cc1 ; c1ccc(-c2ccc3c4c5oc6ccccc6c5ccc4n(-c4nc(-c5ccccc5)c5ccccc5n4)c3c2)cc1 ; c1ccc(-c2cccc3c2sc2c3ccc3c2c2ccccc2n3-c2nccc(-c3cccc4c3sc3ccccc34)n2)cc1]

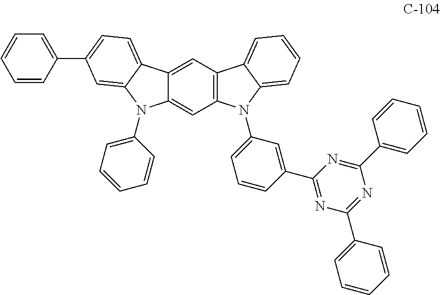 [SMILES: c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1]

The compounds represented by formula 1 can be prepared according to the following reaction scheme 1, but not limited thereto. In addition, modifying the synthetic method is obvious to a person skilled in the art.

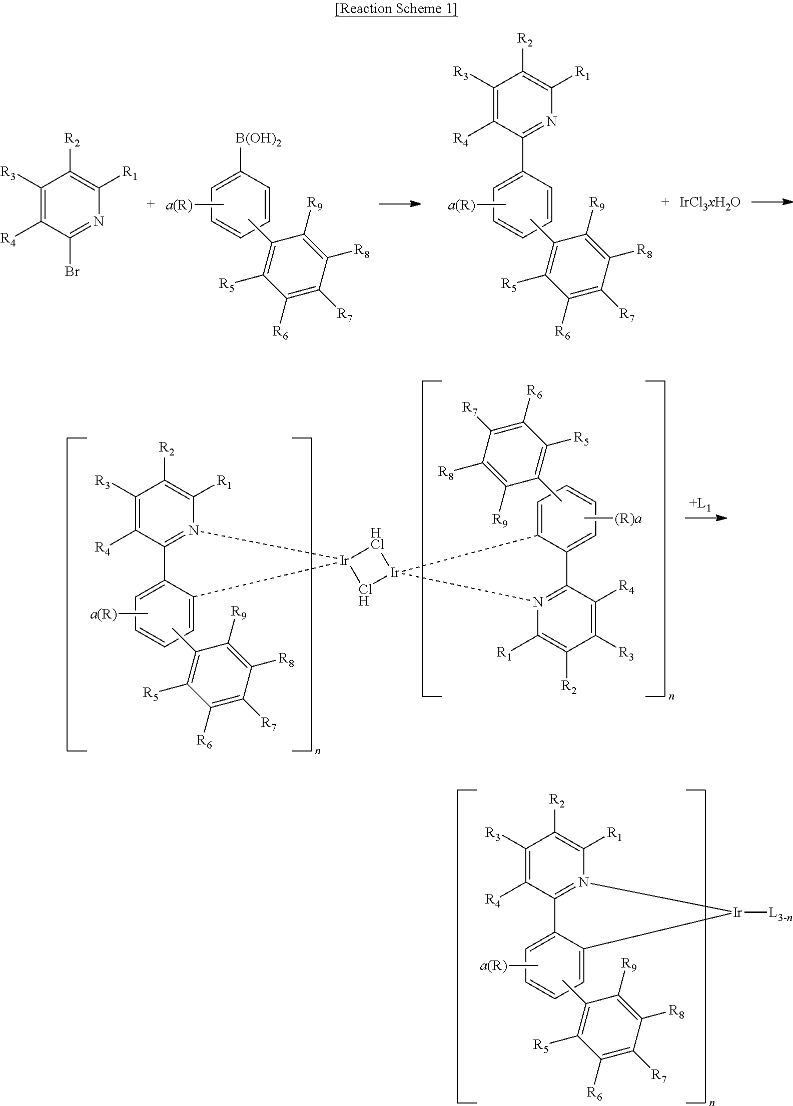

wherein L, R, R₁ to R₉, n, and a are as defined in formula 1 above.

Specifically, said organic electroluminescent device comprises a first electrode; a second electrode; and at least one organic layer between said first and second electrodes. Said organic layer comprises a light-emitting layer, and said light-emitting layer comprises a combination of one or more dopant compounds represented by formula 1, and one or more host compounds represented by formula 2.

Said light-emitting layer is a layer which emits light, and it may be a single layer, or it may be a multi layer of which two or more layers are laminated. The light-emitting layer can also inject/transfer electrons/holes, besides emitting light. The doping concentration, the proportion of the dopant compound to the host compound may be preferably less than 20 wt %.

Another embodiment of the present invention provides a dopant and host combination of one or more dopant compounds represented by formula 1, and one or more host compounds represented by formula 2, and an organic EL device comprising the dopant and host combination.

Still another embodiment of the present invention provides an organic layer consisting of the combination of one or more dopant compounds represented by formula 1, and one or more host compounds represented by formula 2. Said organic layer comprises plural layers. Said dopant compound and said host compound can be comprised in the same layer, or can be comprised in different layers. In addition, the present invention provides an organic EL device comprising the organic layer.

In the organic electroluminescent device according to the present invention, a mixed region of an electron transport compound and an reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting white light.

Hereinafter, the compound, the preparation method of the compound, and the luminescent properties of the device will be explained in detail with reference to the following examples. However, these are just for exemplifying the embodiment of the present invention, so the scope of the present invention cannot be limited thereto.

### Example 1

### Preparation of Compound D-5

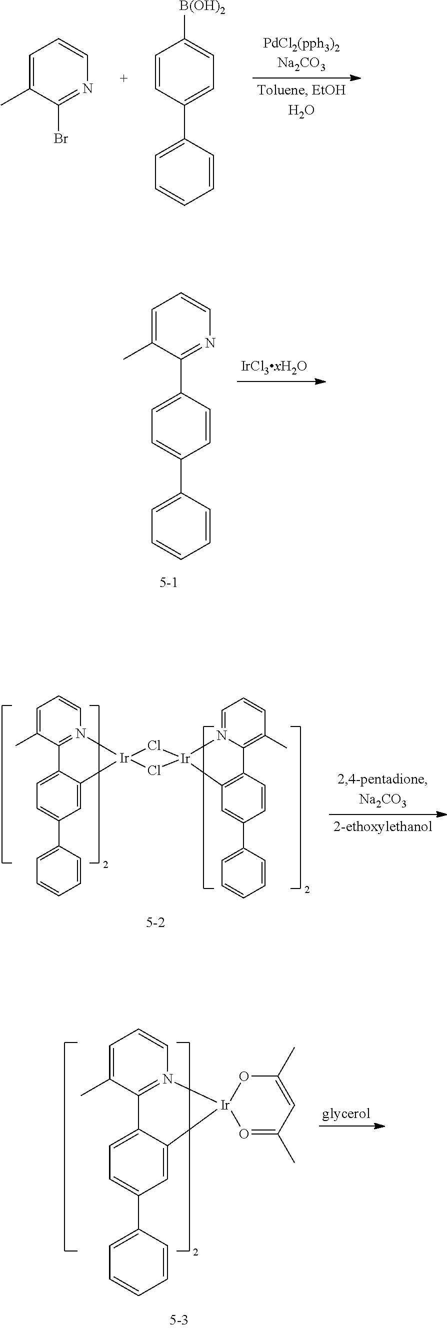

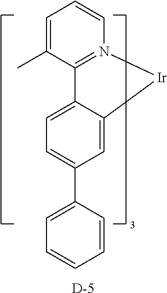

Preparation of Compound 5-1

After adding 4-biphenyl boronic acid 12 g (64 mmol), 2-bromo-3-methylpyridine 10 g (58 mmol), PdCl₂(PPh₃)₂ 1.2 g (1.7 mmol), and Na₂CO₃ 10 g (94 mmol) to a mixture solvent of toluene 100 mL, ethanol 50 mL, and H₂O 50 mL, the mixture was stirred at 120° C. for 4 hours. Then, the reactant mixture was worked up with ethylacetate (EA)/H₂O, moisture was removed with MgSO₄, and the remaining product was distilled under reduced pressure. Then, the product was purified using column chromatography with methylenechloride (MC):hexane (Hex) to obtain white solid compound 5-1 14 g (70%).

Preparation of Compound 5-2

After adding compound 5-1 10 g (41 mmol), and IrCl₃.xH₂O 5 g (17 mmol) to a mixture solvent of 2-ethoxyethanol 120 mL, and H₂O 40 mL, the mixture was stirred at 120° C. for 24 hours under reflux. After the reaction was completed, the mixture was washed using H₂O/MeOH/Hex, and dried to obtain compound 5-2 10 g (75%).

Preparation of Compound 5-3

After adding compound 5-2 10 g (7.0 mmol), 2,4-pentanedion 1.4 g (14 mmol), and Na₂CO₃ 3.7 g (34.7 mmol) to 2-ethoxyethanol 120 mL, the mixture was stirred at 110° C. for 12 hours. After the reaction was completed, the produced solid was washed using H₂O/MeOH/Hex. After sufficiently drying, the product was dissolved with CHCl₃, and purified using column chromatography with MC/Hex to obtain compound 5-3 7.5 g (68%).

Preparation of Compound D-5

After adding glycerol to a mixture of compound 5-3 5 g (6.25 mmol), and compound 5-1 3.1 g (12.4 mmol), the mixture was stirred for 16 hours under reflux. After the reaction was conducted, the obtained solid was filtered, washed using H₂O/MeOH/Hex, and dried. After sufficiently drying, the product was dissolved with CHCl₃, and purified using column chromatography with MC/Hex to obtain compound D-5 3.8 g (64%).

### Example 2

### Preparation of Compound D-9

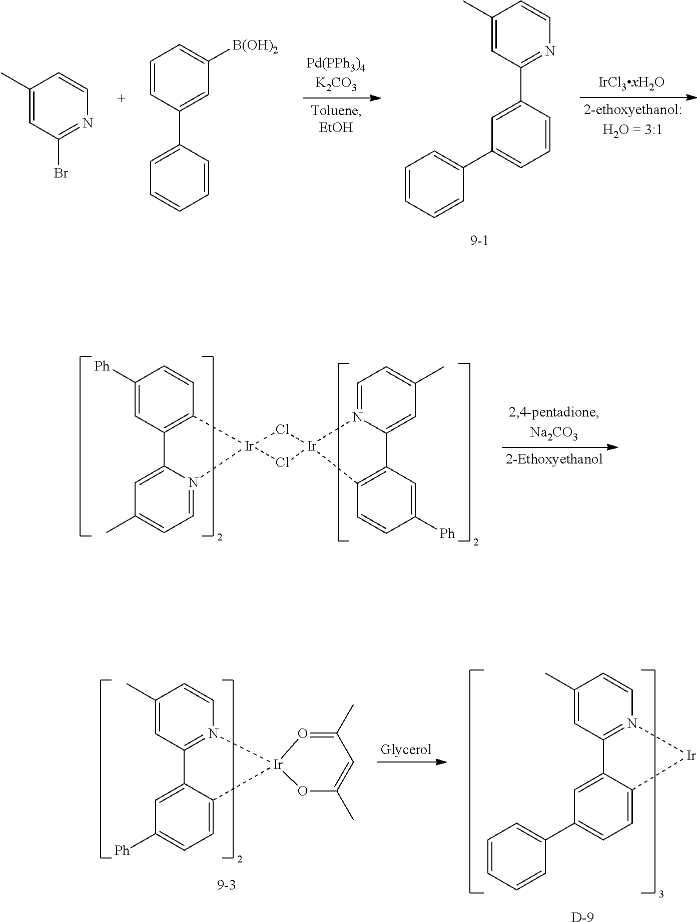

Preparation of Compound 9-1

After adding 3-biphenyl boronic acid 35 g (174 mmol), 2-bromo-4-methylpyridine 20 g (116 mmol), Pd(PPh₃)₄ 4 g (3.5 mmol), and 2 M K₂CO₃ 200 mL (400 mmol) to a mixture solvent of toluene 400 mL, and ethanol 400 mL, the mixture was stirred at 100° C. for 3 hours. Then, the reactant mixture was worked up with EA/H₂O, moisture was removed with MgSO₄, and the remaining product was distilled under reduced pressure. Then, the product was purified using column chromatography with MC/Hex to obtain white solid compound 9-1 18 g (63%).

Preparation of Compound 9-2

After adding compound 9-1 7.6 g (31 mmol), and IrCl₃.xH₂O 4.2 g (14 mmol) to a mixture solvent of 2-ethoxyethanol 110 mL, and H₂O 37 mL, the mixture was stirred at 130° C. for 24 hours. After the reaction was completed, the reactant was cooled to room temperature, washed with water and MeOH, and dried to obtain compound 9-2 8 g (80%).

Preparation of Compound 9-3

After adding compound 9-2 7 g (5 mmol), 2,4-pentanedion 1.5 g (15 mmol), and Na₂CO₃ 1.6 g (15 mmol) to 2-ethoxyethanol 80 mL, the reaction was conducted at 110° C. for 3 hours. After the reaction was completed, the produced solid was purified using column chromatography to obtain compound 9-3 5 g (70%).

Preparation of Compound D-9

After adding glycerol to a mixture of compound 9-3 4 g (5 mmol), and compound 9-1 2.5 g (10 mmol), the mixture was stirred at 220° C. for 24 hours under reflux. After the reaction was completed, the produced solid was purified using column chromatography to obtain compound D-9 4 g (80%).

### Example 3

### Preparation of Compound D-28

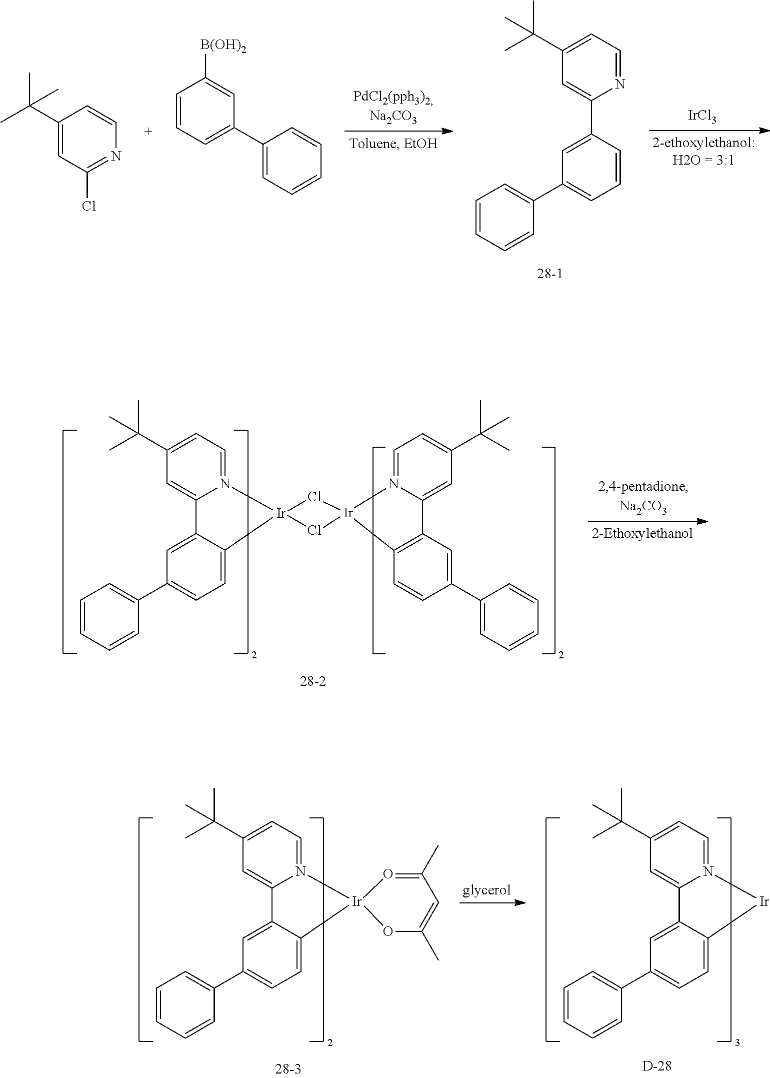

Compound 28-1 to compound 28-3 were prepared in the same manner as the synthetic method of compound 9-1 to compound 9-3 of Example 2.

Preparation of Compound D-28

After adding glycerol to a mixture of compound 28-3 4.5 g (5.2 mmol), and compound 28-1 3.0 g (10.4 mmol), the mixture was stirred for 16 hours under reflux. After the reaction was conducted, the obtained solid was filtered, washed using H₂O/MeOH/Hex, and dried. After sufficiently drying, the product was dissolved with CHCl₃, and purified using column chromatography with MC/Hex to obtain compound D-28 1.8 g (33%).

The detailed data of the dopant compounds prepared in Examples 1 to 3, and the dopant compounds easily prepared using Examples 1 to 3 are shown in table 1 below.

TABLE 1

| | Yield | UV spectrum | PL spectrum | Melting Point |
|---|---|---|---|---|
| Compound | (%) | (nm) | (nm) | (° C.) |
| D-2 | 63 | 322 | 540 | 310 |
| D-5 | 64 | 326 | 534 | over 400 |
| D-9 | 80 | 286 | 513 | over 400 |
| D-10 | 56 | 269 | 517 | 389 |
| D-14 | 23 | 324 | 510 | 335 |
| D-18 | 68 | 294 | 515 | 350 |
| D-28 | 27 | 296 | 511.94 | over 400 |

### Example 4

### Preparation of Compound C-11

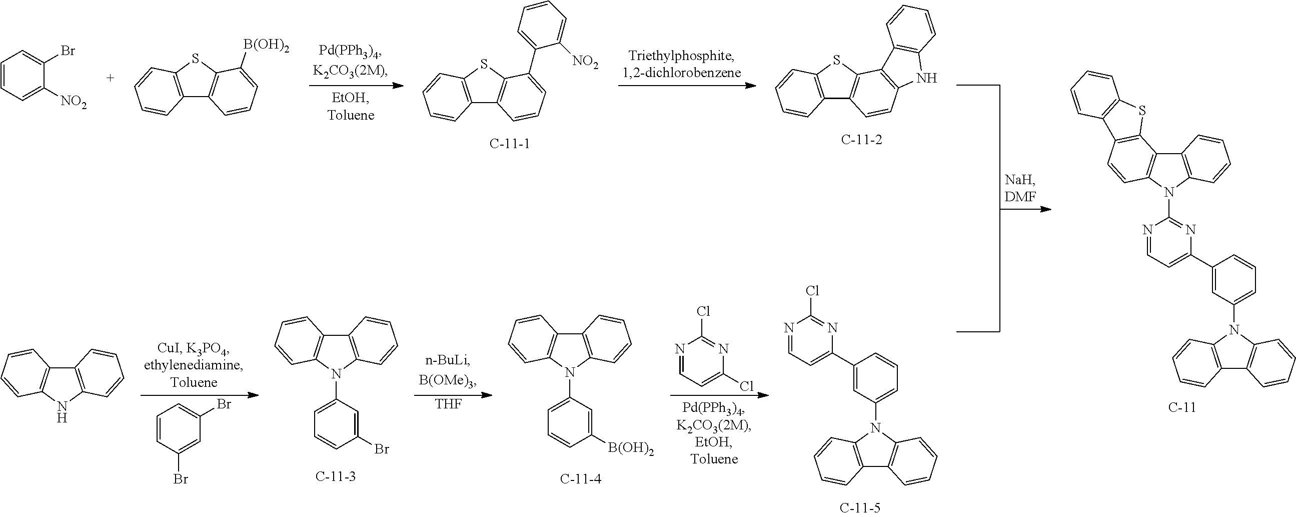

Preparation of Compound C-11-1

After mixing 1-bromo-2-nitrobenzene 85 g (0.42 mol), dibenzo[b,d]thiophen-4-yl boronic acid 80 g (0.35 mol), Pd(PPh₃)₄ 20 g (0.018 mol), K₂CO₃ 116 g (1.0 mol), toluene 1700 mL, ethanol 440 mL, and H₂O 440 mL in a round bottom flask, the mixture was stirred at 120° C. for 12 hours. After the reaction was completed, the mixture was extracted with ethylacetate, and the organic layer was dried with MgSO₄. Then, the remaining product was filtered, solvent was removed under reduced pressure, and the remaining product was separated with a column to obtain white solid compound C-11-1 93 g (87%).

Preparation of Compound C-11-2

After mixing compound C-11-1 88 g (0.29 mol), 1,2-dichlorobenzene 960 mL (0.4 M), and triethylphosphite 960 mL in a round bottom flask under anhydrous condition, the mixture was stirred at 90° C. for 12 hours. After the reaction was completed, the mixture was distilled to remove triethylphosphite, and the remaining product was separated with a column to obtain white solid compound C-11-2 40 g (70%).

Preparation of Compound C-11-3

After mixing 9H-carbazole 30 g (0.18 mol), 1,3-dibromobenzene 85 g (0.36 mol), CuI 34 g (0.18 mol), K₃PO₄ 114 g (0.54 mol), and toluene 1200 mL in a round bottom flask, the mixture was stirred at 120° C. for 10 minutes. Then, ethylenediamine 24 mL (0.36 mol) was added to the mixture, and the mixture was stirred at 120° C. for 12 hours. After the reaction was completed, the mixture was extracted with ethylacetate (EA), and the organic layer was dried with MgSO₄. Then, the remaining product was filtered, solvent was removed under reduced pressure, and the remaining product was separated with a column to obtain white solid compound C-11-3 30 g (52%).

Preparation of Compound C-11-4

After dissolving compound C-11-3 25 g (80.85 mmol) in tetrahydrofuran (THF), n-buLi 42 mL (105.10 mmol, 2.5 M in hexane) was slowly added to the mixture at −78° C. After 1 hour, trimethylborate 14.42 mL (129.3 mmol) was added to the mixture. Then, the mixture was stirred for 12 hours at room temperature, and distilled water was added to the mixture. Then, the mixture was extracted with EA, dried with MgSO₄, distilled under reduced pressure, and recrystallized with MC and hexane to obtain compound C-11-4 15 g (58%).

Preparation of Compound C-11-5

After adding compound C-11-4 20 g (72.96 mmol), 2,4-dichloropyrimidine 9.8 g (80.25 mmol), Pd(PPh₃)₄ 2.28 g (2.18 mmol), 2 M K₂CO₃ 80 mL, toluene 150 mL, and ethanol 50 mL in a flask, the mixture was stirred for 5 hours under reflux. Then, the mixture was cooled to room temperature, and distilled water was added to the mixture. Then, the mixture was extracted with EA, dried with MgSO₄, distilled under reduced pressure, and recrystallized with EA and methanol to obtain compound C-11-5 9.8 g (80%).

Preparation of Compound C-11

After mixing NaH 0.9 g (60%, 20 mmol), and dimethylformamide (DMF) 50 mL in a 500 mL round bottom flask under anhydrous condition, compound C-11-2 4.69 g (17 mmol) was dissolved in DMF 20 mL, and added into the round bottom flask comprising NaH. After stirring the mixture for 1 hour, compound C-11-5 6.1 g (17 mmol) was dissolved in DMF 100 mL, and added into the flask comprising compound C-11-2. After stirring the mixture for 12 hours, yellow solid was filtered, and recrystallized to obtain compound C-11 3 g (30%).

### Example 5

### Preparation of Compound C-38

Preparation of Compound C-38-1

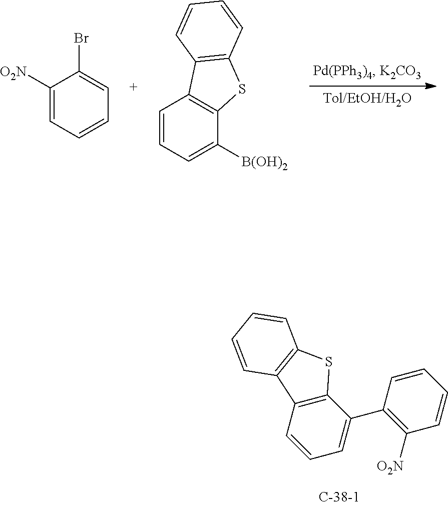 [SMILES: O=[N+]([O-])c1ccccc1Br ; OB(O)c1cccc2c1sc1ccccc12 ; O=[N+]([O-])c1ccccc1-c1cccc2c1sc1ccccc12]

After adding 1-bromo-2-nitrobenzene 50 g (248 mmol), dibenzothiophen-4-boronic acid 62 g (272 mmol), K₂CO₃ 85 g (619 mmol), and Pd(PPh₃)₄ 14 g (12.4 mmol) in a mixture solvent of toluene 900 mL, EtOH 200 mL, and purified water 300 mL, the mixture was stirred for a day under reflux. After the reaction was completed, the mixture was cooled to room temperature, and extracted with distilled water and EA. Then, the organic layer was distilled under reduced pressure, and separated with a column using MC/Hex to obtain compound C-38-1 60 g (80%).

Preparation of Compound C-38-2

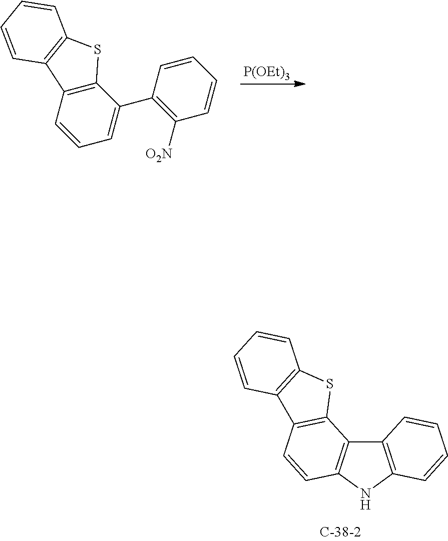

After adding P(OEt)₃ 1 L to compound C-38-1 130 g (426 mmol), the mixture was stirred at 150° C. for a day. After the reaction was completed, the mixture was concentrated under reduced pressure, extracted with MC, and the organic layer was concentrated. Then, the obtained product was separated with a column using MC/Hex to obtain compound C-38-2 46 g (40%).

Preparation of Compound C-38-3

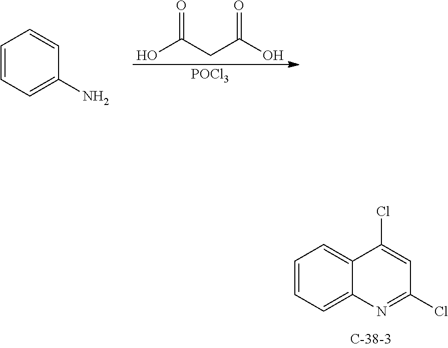

After slowly adding dropwise POCl₃ to a mixture of aniline 15 mL (169 mmol), and malonic acid 25 g (492 mmol), the mixture was stirred for a day under reflux. After adding the reactant mixture slowly to iced water, the mixture was neutralized using 5 M NaOH. Then, the produced solid was extracted with distilled water and MC, the organic layer was distilled under reduced pressure, and separated with a column using MC/Hex to obtain compound C-38-3 17 g (53%).

Preparation of Compound C-38-4

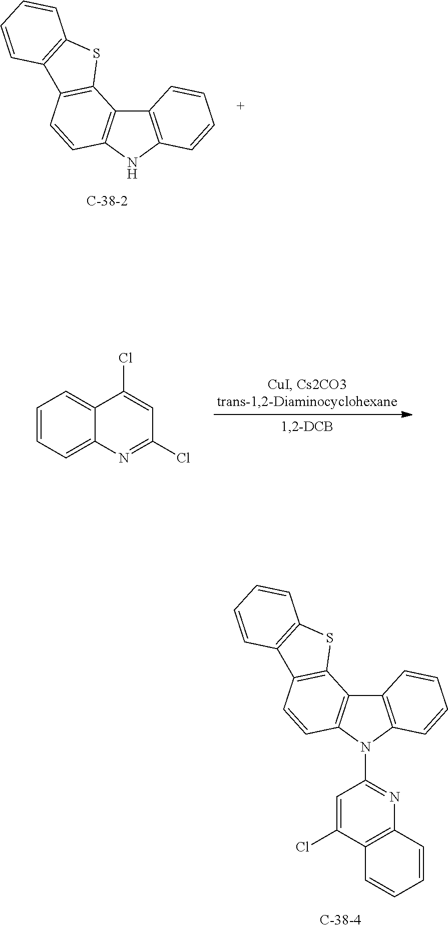 [SMILES: c1ccc2c(c1)[nH]c1ccc3c4ccccc4sc3c12 ; Clc1cc(Cl)c2ccccc2n1 ; Clc1cc(-n2c3ccccc3c3c4sc5ccccc5c4ccc32)nc2ccccc12]

After adding 1,2-dichlorobenzene to a mixture of compound C-38-2 6.2 g (31.4 mmol), compound C-38-3 8.6 g (31.4 mmol), CuI 12 g (63 mmol), Cs₂CO₃ 31 g (95 mmol), and trans-1,2-diaminocyclohexane, the mixture was stirred for a day under reflux. After the reaction was completed, the mixture was concentrated under reduced pressure, extracted with MC, and the organic layer was concentrated. Then, the obtained product was separated with a column using MC/Hex to obtain compound C-38-4 7.4 g (55%).

Preparation of Compound C-38

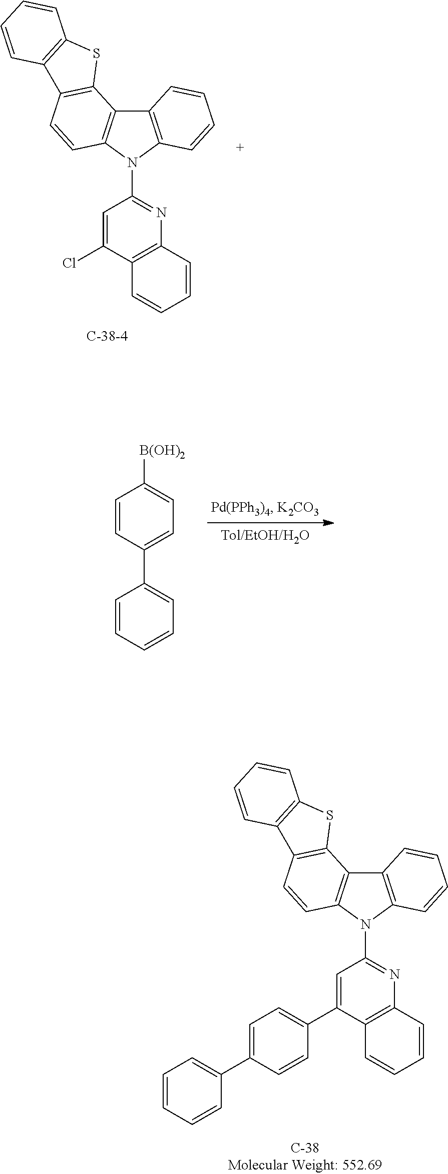 [SMILES: Clc1cc(-n2c3ccccc3c3c4sc5ccccc5c4ccc32)nc2ccccc12 ; OB(O)c1ccc(-c2ccccc2)cc1 ; c1ccc(-c2ccc(-c3cc(-n4c5ccccc5c5c6sc7ccccc7c6ccc54)nc4ccccc34)cc2)cc1]

After adding compound C-38-4 6.8 g (15.6 mmol), 4-biphenyl boronic acid 6.2 g (31 mmol), K₂CO₃ 5.4 g (38.9 mmol), and Pd(PPh₃)₄ 1.8 g (1.6 mmol) in a mixture solvent of toluene 100 mL, EtOH 15 mL, and purified water 20 mL, the mixture was stirred for a day under reflux. After the reaction was completed, the mixture was cooled to room temperature, and extracted with distilled water and MC. Then, the organic layer was distilled under reduced pressure, and separated with a column using MC/Hex to obtain compound C-38 3.5 g (41%).

The detailed data of the host compounds prepared in Examples 4 and 5, and the host compounds easily prepared using Examples 4 and 5 are shown in table 2 below.

TABLE 2

| | | PL spectrum | Melting | | |
|---|---|---|---|---|---|
| | Yield | (in toluene, | Point | MS/EIMS | |
| Compound | (%) | nm) | (° C.) | Found | Calculated |
| C-1 | 57.5 | 506 | 379 | 719.85 | 719.20 |
| C-2 | 28 | 492 | 298 | 757.2 | 755.9 |
| C-3 | 45 | 495 | 265 | 756.2 | 754.9 |
| C-4 | 28 | Not dissolved | 323 | 746.80 | 745.80 |
| C-5 | 42 | 475 | 288 | 745.90 | 744.90 |
| C-6 | 43 | 505 | 346 | 756.91 | 755.91 |
| C-7 | 19.3 | 420(MC) | 329 | 579.7 | 579.18 |
| C-8 | 57 | 420 | 295 | 563.6 | 563.2 |
| C-9 | 54 | 406 | 339 | 579.7 | 579.18 |
| C-10 | 36 | 388 | 198 | 603 | 602.73 |
| C-11 | 40 | 471 | 270 | 593 | 592.71 |
| C-12 | 60 | 468 | 280 | 593 | 592.71 |
| C-13 | 18 | 469 | 282 | 577 | 576.64 |
| C-14 | 40 | 474 | 284 | 669 | 668.81 |
| C-15 | 30 | 469 | 293 | 669 | 668.81 |
| C-16 | 18 | 466 | 186 | 630 | 629.79 |
| C-17 | 50 | 386 | 218 | 590 | 589.73 |
| C-18 | 67.3 | — | 249 | 581 | 580.90 |
| C-19 | 67 | 451 | 249 | 620 | 619.7 |
| C-20 | 49 | 491(MC) | 274 | 580 | 579.7 |
| C-21 | 26 | 460 | 245 | 656 | 655.81 |
| C-22 | 34 | — | 294 | 656 | 655.81 |
| C-23 | 67 | 486 | 277 | 656 | 655.81 |
| C-24 | 53 | 465 | 272 | 656 | 655.81 |
| C-25 | 34.3 | 396 | 222 | 656 | 655.81 |

### Device Example 1

### Production of an OLED Device Using the Organic Electroluminescent Compound According to the Present Invention

An OLED device was produced using the light emitting material according to the present invention. A transparent electrode indium tin oxide (ITO) thin film (15 Ω/sq) on a glass substrate for an organic light-emitting diode (OLED) device (Samsung Corning, Republic of Korea) was subjected to an ultrasonic washing with trichloroethylene, acetone, ethanol and distilled water, sequentially, and then was stored in isopropanol. Then, the ITO substrate was mounted on a substrate holder of a vacuum vapor depositing apparatus. N¹,N^{1′}-([1,1′-biphenyl]-4,4′-diyl)bis(N¹-(naphthalen-1-yl)-N⁴,N⁴-diphenylbenzen-1,4-diamine) was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a hole injection layer having a thickness of 60 nm on the ITO substrate. Then, N,N′-di(4-biphenyl)-N,N′-di(4-biphenyl)-4,4′-diaminophenyl was introduced into another cell of said vacuum vapor depositing apparatus, and was evaporated by applying an electric current to the cell, thereby forming a hole transport layer having a thickness of 20 nm on the hole injection layer. Thereafter, compound C-11 was introduced into one cell of the vacuum vapor depositing apparatus, as a host material, and compound D-9 was introduced into another cell as a dopant. The two materials were evaporated at different rates and were deposited in a doping amount of 15 wt % based on the total amount of the host and dopant to form a light-emitting layer having a thickness of 30 nm on the hole transport layer. Then, 2-(4-(9,10-di(naphthalen-2-yl)anthracen-2-yl)phenyl)-1-phenyl-1H-benzo[d]imidazole was introduced into one cell and lithium quinolate was introduced into another cell. The two materials were evaporated at the same rate and were deposited in a doping amount of 50 wt % each to form an electron transport layer having a thickness of 30 nm on the light-emitting layer. Then, after depositing lithium quinolate as an electron injection layer having a thickness of 2 nm on the electron transport layer, an Al cathode having a thickness of 150 nm was deposited by another vacuum vapor deposition apparatus on the electron injection layer. Thus, an OLED device was produced. All the materials used for producing the OLED device were purified by vacuum sublimation at 10⁻⁶ torr prior to use.

The produced OLED device showed a green emission having a luminance of 5030 cd/m² and a current density of 13.97 mA/cm² at a driving voltage of 5.0 V.

### Device Example 2

### Production of an OLED Device Using the Organic Electroluminescent Compound According to the Present Invention

An OLED device was produced in the same manner as in Device Example 1, except for using compound C-17 as a host, and using compound D-28 as a dopant of the light emitting material.

The produced OLED device showed a green emission having a luminance of 2060 cd/m² and a current density of 4.63 mA/cm² at a driving voltage of 3.2 V.

### Comparative Example 1

### Production of an OLED Device Using Conventional Organic Electroluminescent Material

An OLED device was produced in the same manner as in Device Example 1, except for using 4,4′-N,N′-dicarbazol-biphenyl as a host, compound Ir(ppy)₃ as a dopant to form a light-emitting layer having a thickness of 30 nm on the hole transport layer; and depositing aluminum(III)bis(2-methyl-8-quinolinato)4-phenylphenolate to form a hole blocking layer having a thickness of 10 nm.

The produced OLED device showed a green emission having a luminance of 3000 cd/m² and a current density of 9.8 mA/cm² at a driving voltage of 7.5 V.

As shown above, the organic EL device of the present invention contains a light emitting material comprising a specific combination of a dopant and a host compound, and provides improved luminous efficiency at a lower driving voltage than the device using conventional luminous materials. This is because the energy gap is controlled by introducing alkyl and aryl groups to a Ir(ppy)₃ structure which is a conventional dopant compound. By this method, the energy gap of the host compound of the present invention is better combined with the dopant compound of the present invention than that of the conventional host compound, and finally the organic EL device of the present invention provides excellent luminous efficiency.

1. A combination of one or more dopant compound represented by the following formula 1, and one or more host compound represented by the following formula 2: 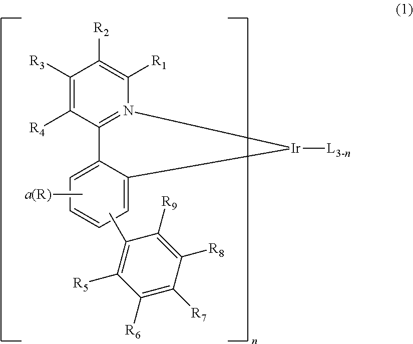
wherein
L is an organic ligand;
R₁ to R₉ each independently represent hydrogen, deuterium, a halogen, a substituted or unsubstituted (C1-C30)alkyl, a substituted or unsubstituted (C3-C30)cycloalkyl, a substituted or unsubstituted (C1-C30)alkoxy, a substituted or unsubstituted (C6-C30)aryl, or a substituted or unsubstituted 3- to 30-membered heteroaryl;
R represents hydrogen, a halogen, a substituted or unsubstituted (C1-C30)alkyl, or a substituted or unsubstituted (C3-C30)cycloalkyl;
a represents an integer of 1 to 3; where a is an integer of 2 or more, each of R are same or different; and
n represents an integer of 1 to 3;
A-D-C (2)
wherein
D represents a single bond, a substituted or unsubstituted (C3-C30)cycloalkylene, a substituted or unsubstituted (C6-C30)arylene, or a substituted or unsubstituted 5- to 30-membered heteroarylene;
A is represented by the following formula 3 or 4: 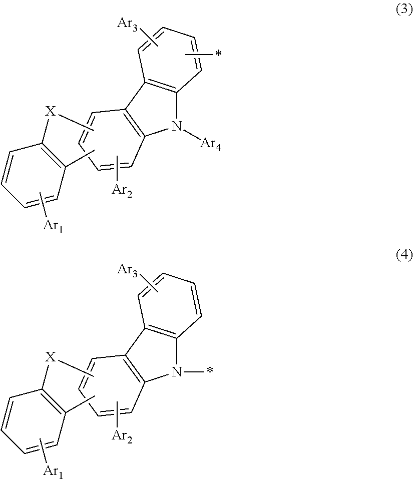
wherein
-* represents a position to which D is linked;
X represents NR₁₀, O, S, or CR₁₁R₁₂;
Ar₁ to Ar₄ each independently represent hydrogen, a substituted or unsubstituted (C1-C30)alkyl, a substituted or unsubstituted (C6-C30)aryl, a substituted or unsubstituted 5- to 30-membered heteroaryl, a substituted or unsubstituted (C3-C30)cycloalkyl, —NR₁₃R₁₄, —SiR₁₅R₁₆R₁₇, —SR₁₈, or —OR₁₉; and
R₁₀ to R₁₉ each independently represent hydrogen, a substituted or unsubstituted (C1-C30)alkyl, or a substituted or unsubstituted (C6-C30)aryl;
C is represented by the following formula 5: 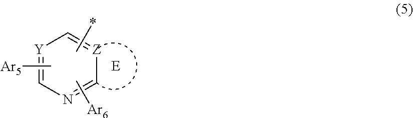
wherein
-* represents a position to which D is linked;
Y and Z each independently represent CH or N;
E ring represents a substituted or unsubstituted benzene ring or is absent;
Ar₅ and Ar₆ each independently represent hydrogen, a substituted or unsubstituted (C6-C30)aryl, a substituted or unsubstituted 5- to 30-membered heteroaryl, a substituted or unsubstituted (C6-C30)cycloalkyl, —NR₂₁R₂₂, —SiR₂₃R₂₄R₂₅, —SR₂₆, or —OR₂₇; and
R₂₁ to R₂₇ each independently represent hydrogen, a substituted or unsubstituted (C1-C30)alkyl, a substituted or unsubstituted (C6-C30)aryl, or a substituted or unsubstituted 5- to 30-membered heteroaryl.

2. The combination according to claim 1, wherein the compound represented by formula 1 is represented by the following formula 6 or 7: 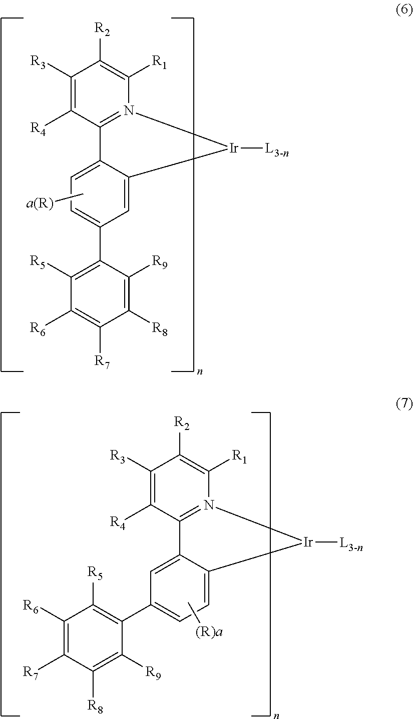
wherein R, R₁ to R₉, L, n and a are as defined in claim 1.

3. The combination according to claim 1, wherein L in formula 1 is selected from the group consisting of: 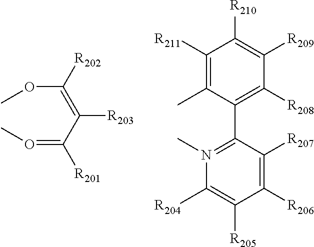
wherein R₂₀₁ to R₂₁₁ each independently represent hydrogen, deuterium, a halogen, a substituted or unsubstituted (C1-C30)alkyl, or a substituted or unsubstituted (C3-C30)cycloalkyl.

4. The combination according to claim 1, wherein D in formula 2 is a single bond, or is selected from the group consisting of: 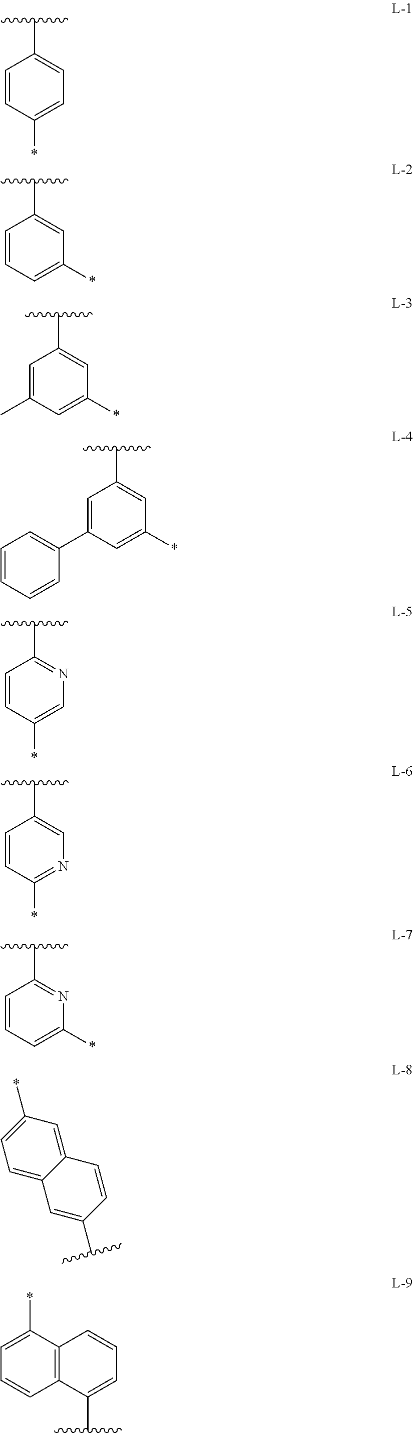
wherein -* represents a position to which C is linked, and [symbol] represents a position to which A is linked.

5. The combination according to claim 1, wherein A in formula 2 is selected from the group consisting of:

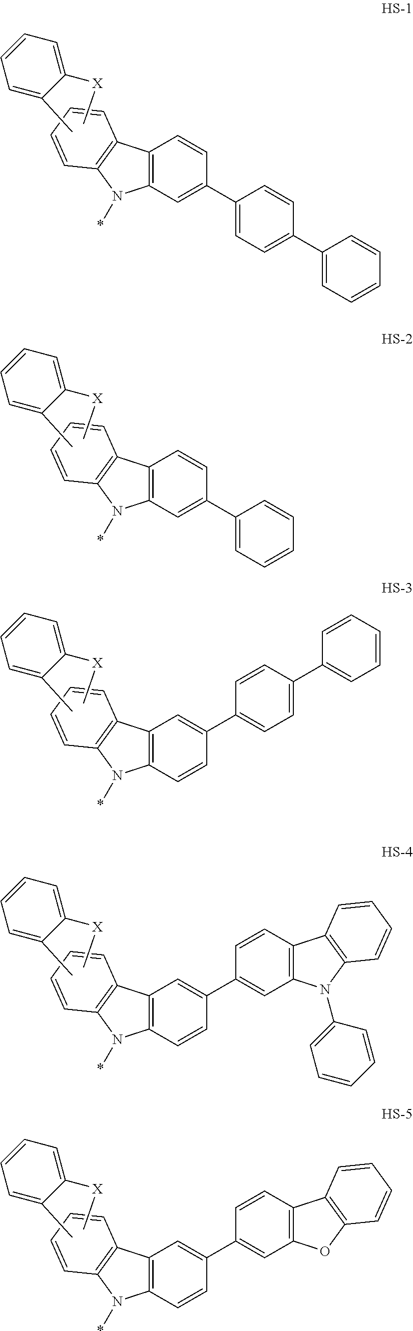

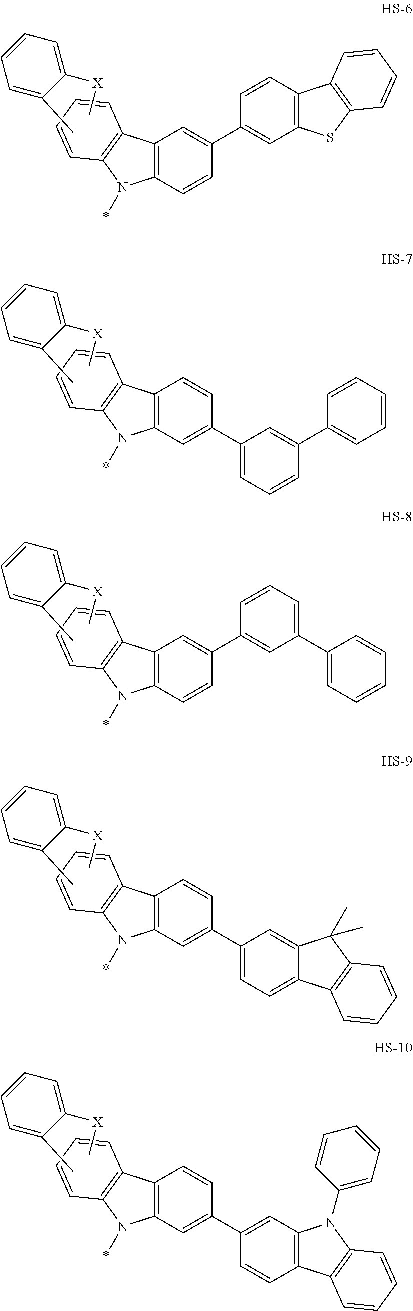

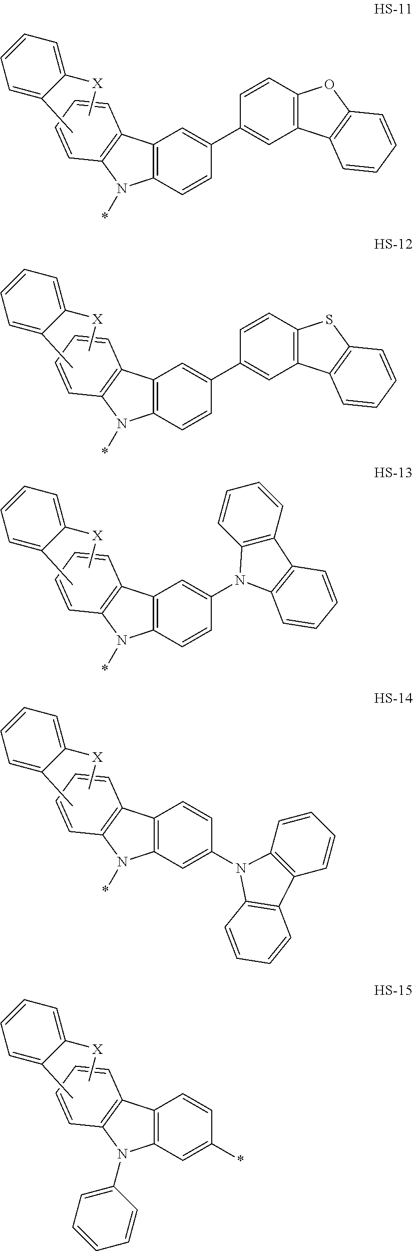

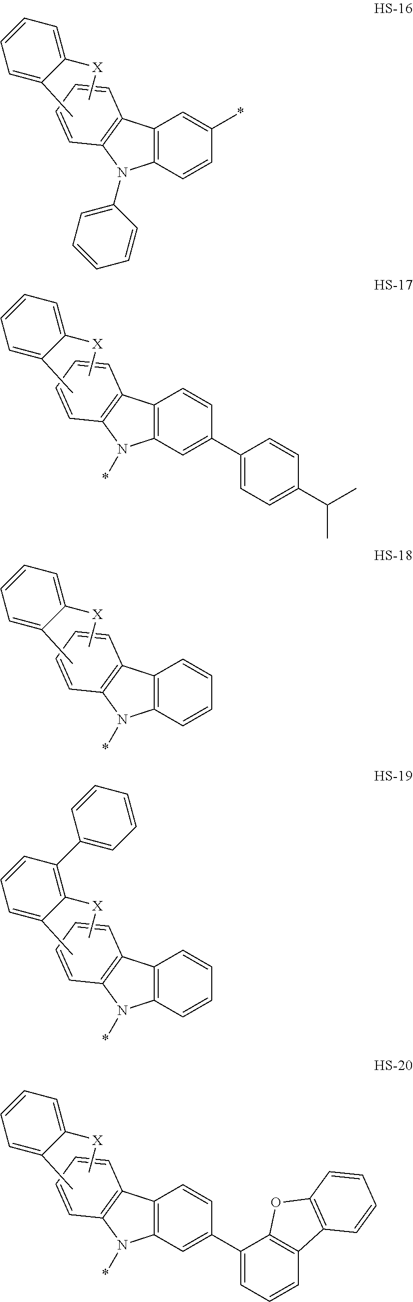

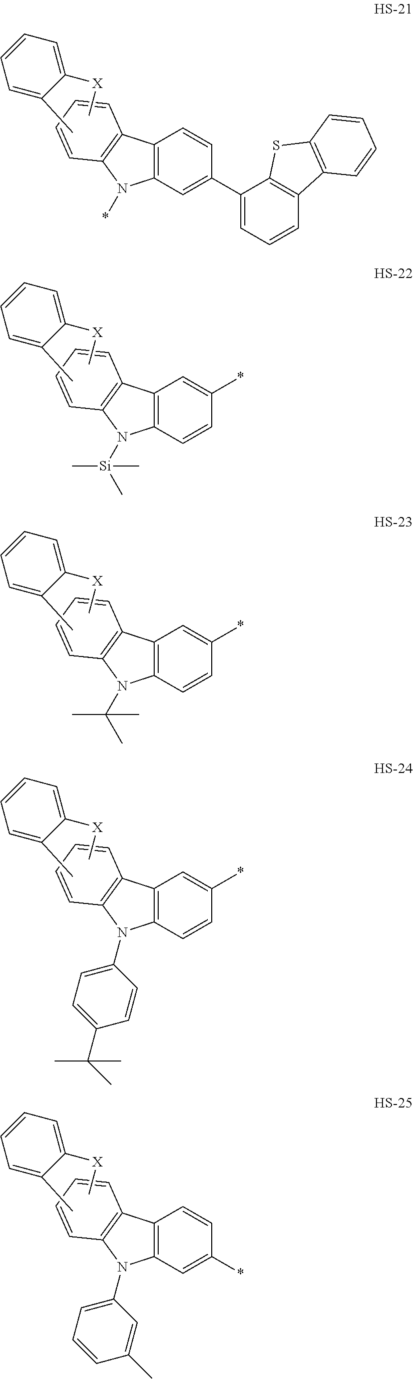

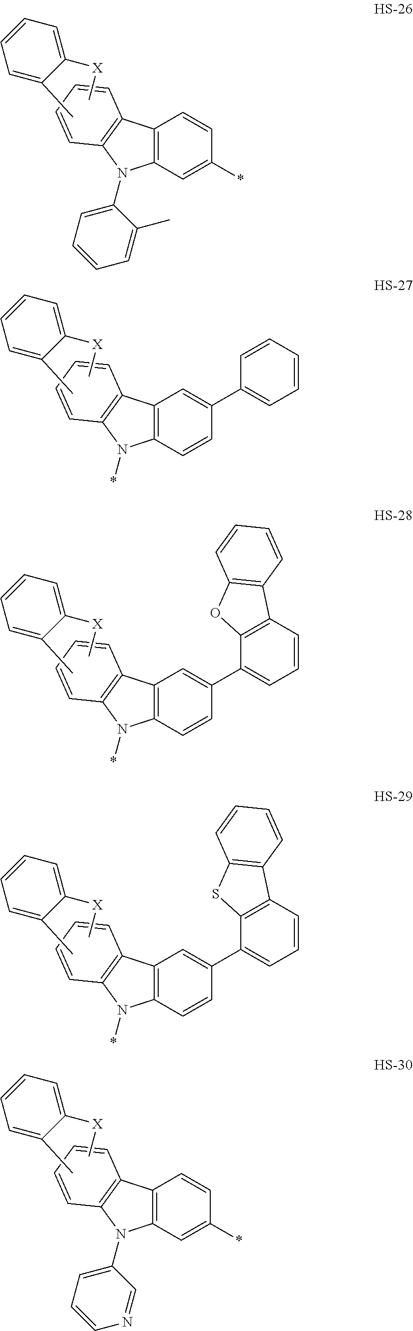

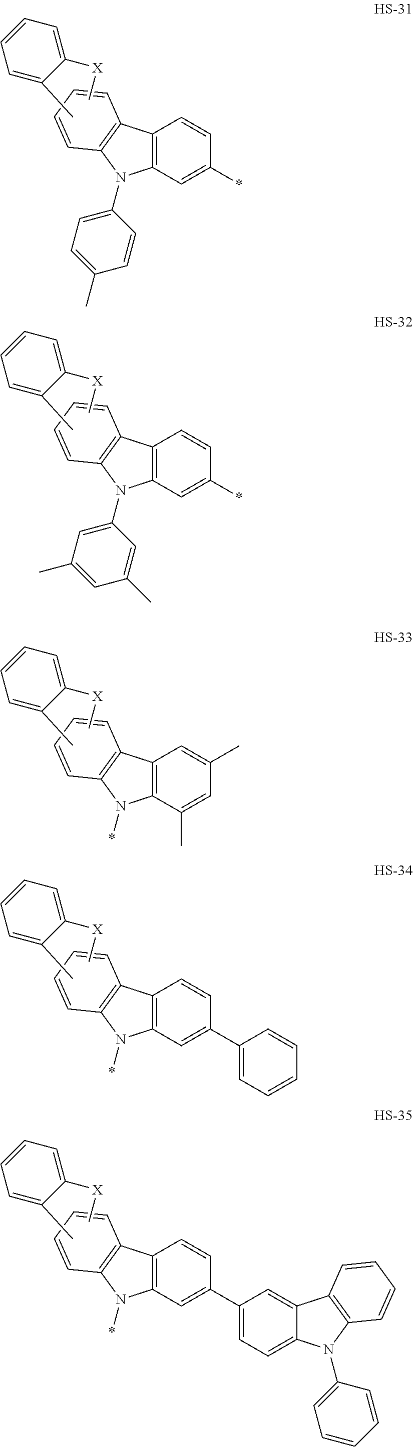

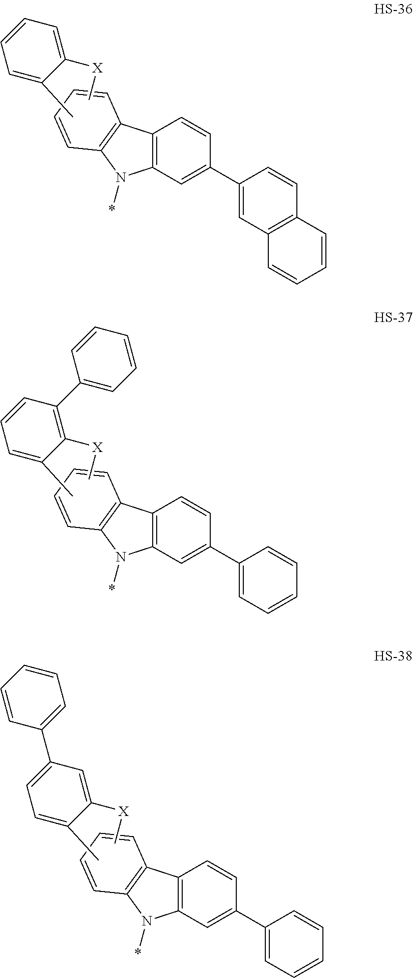

6. The combination according to claim 1, wherein C in formula 2 is selected from the group consisting of:

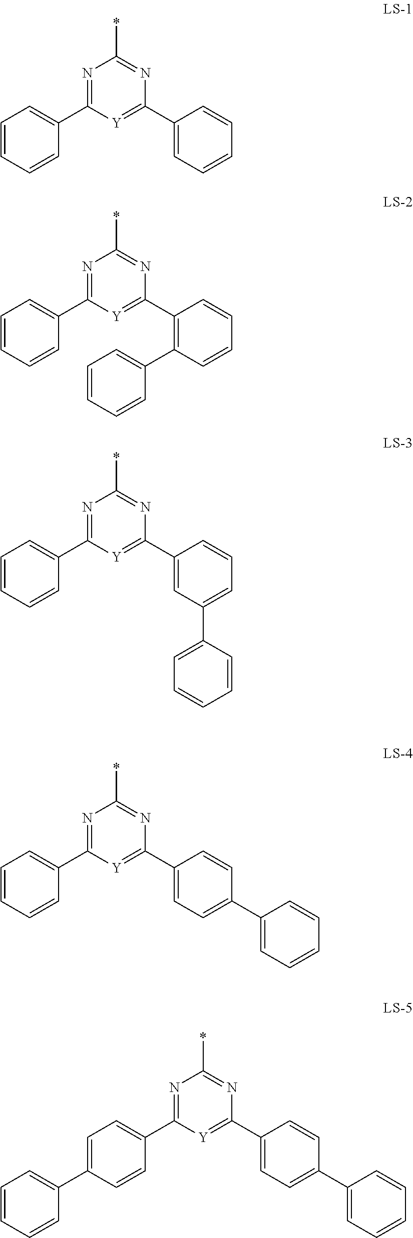

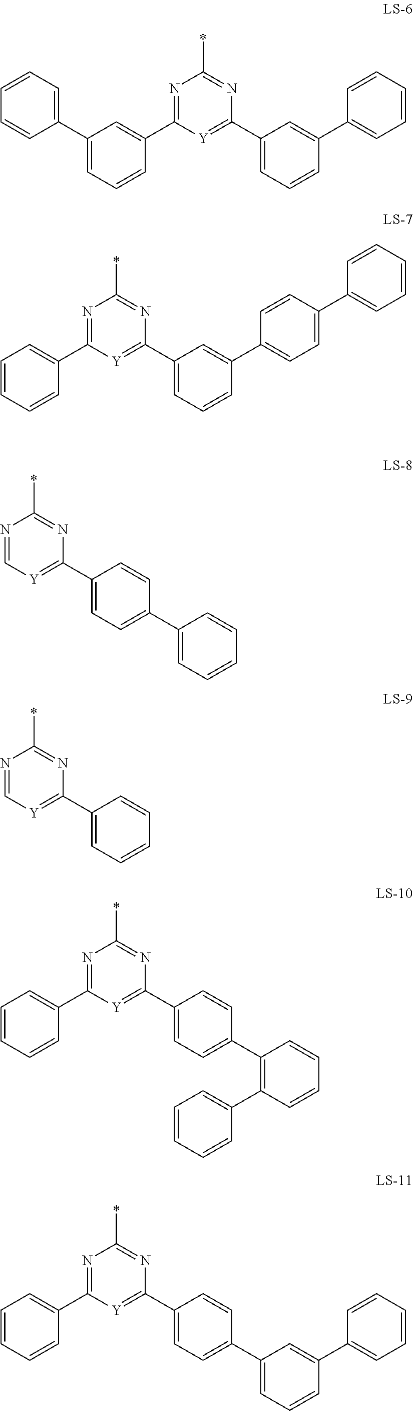

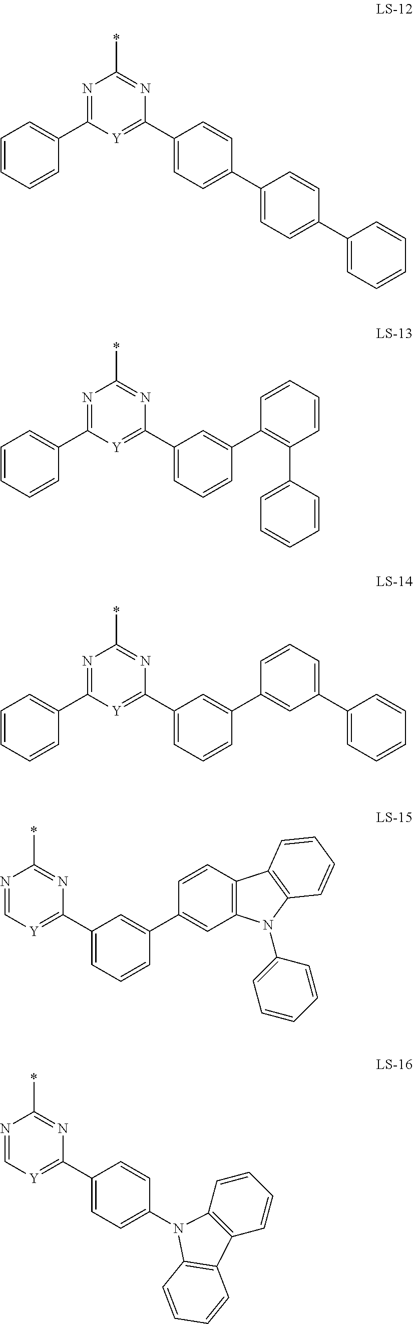

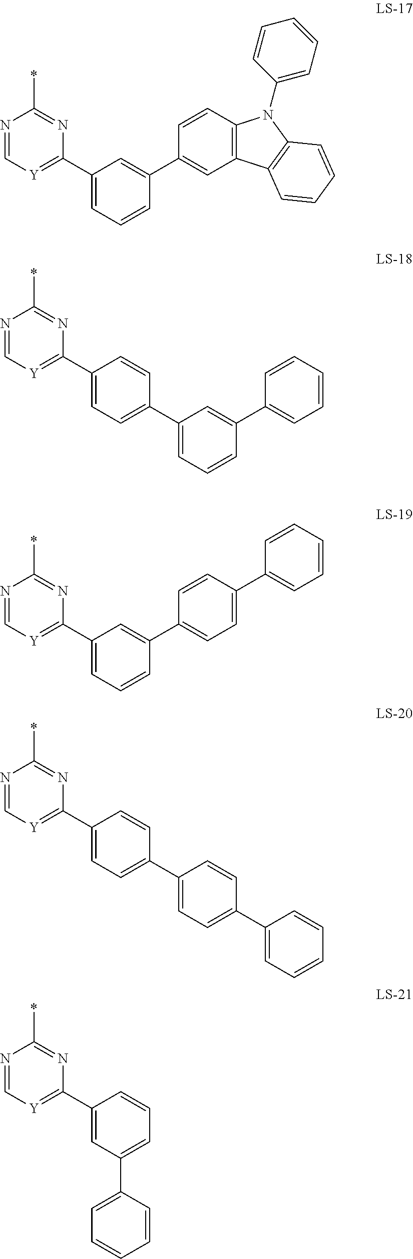

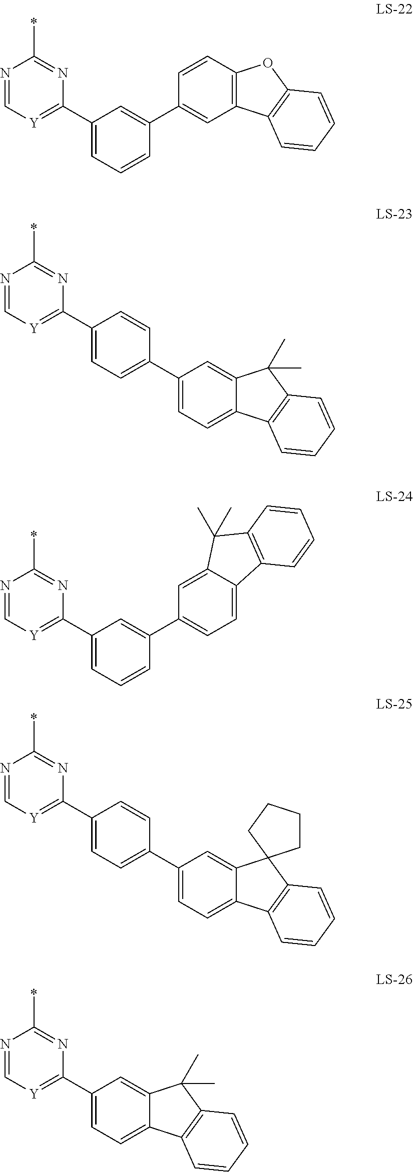

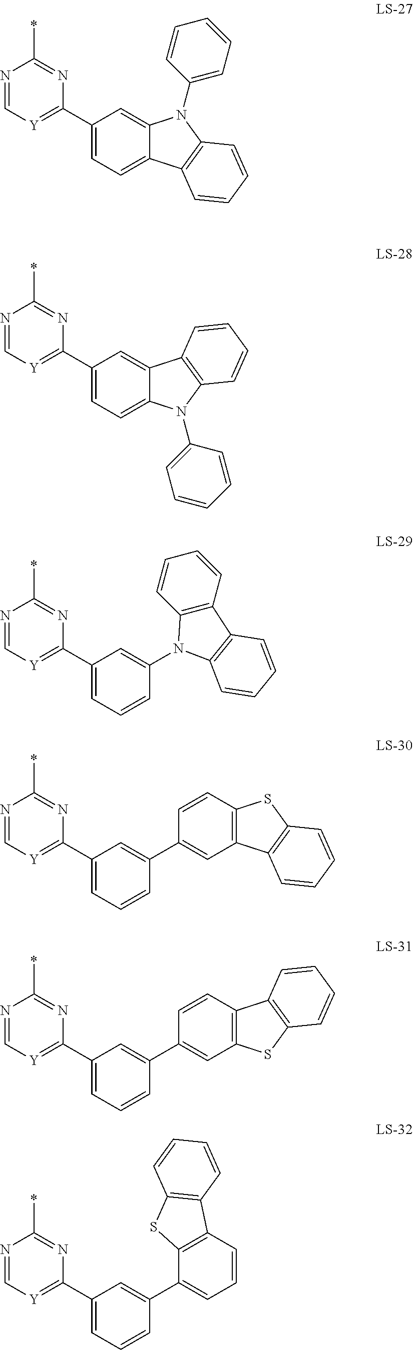

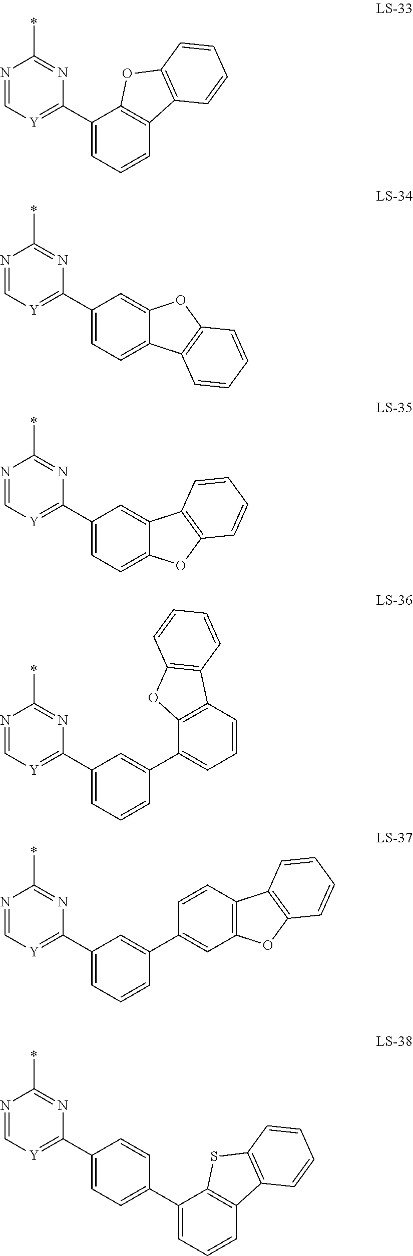

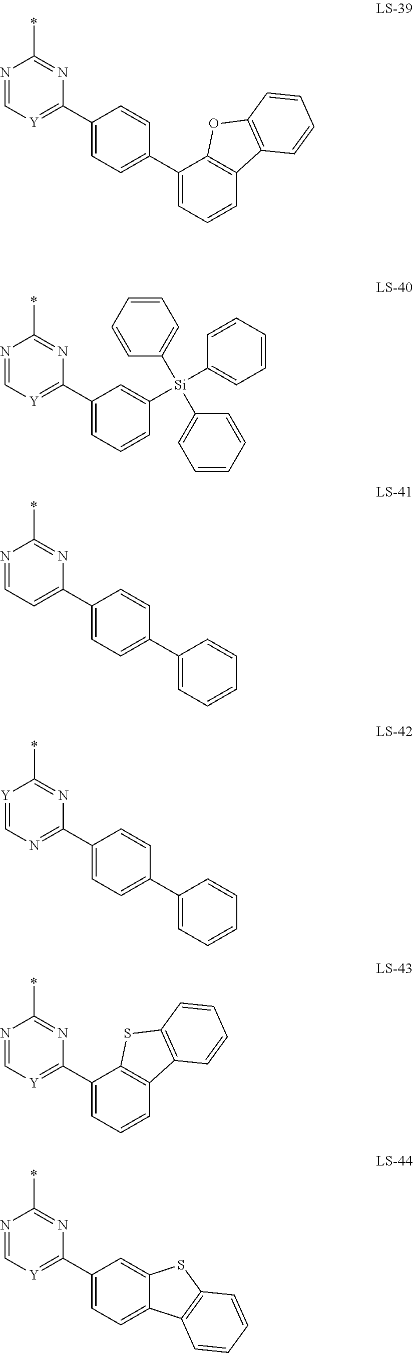

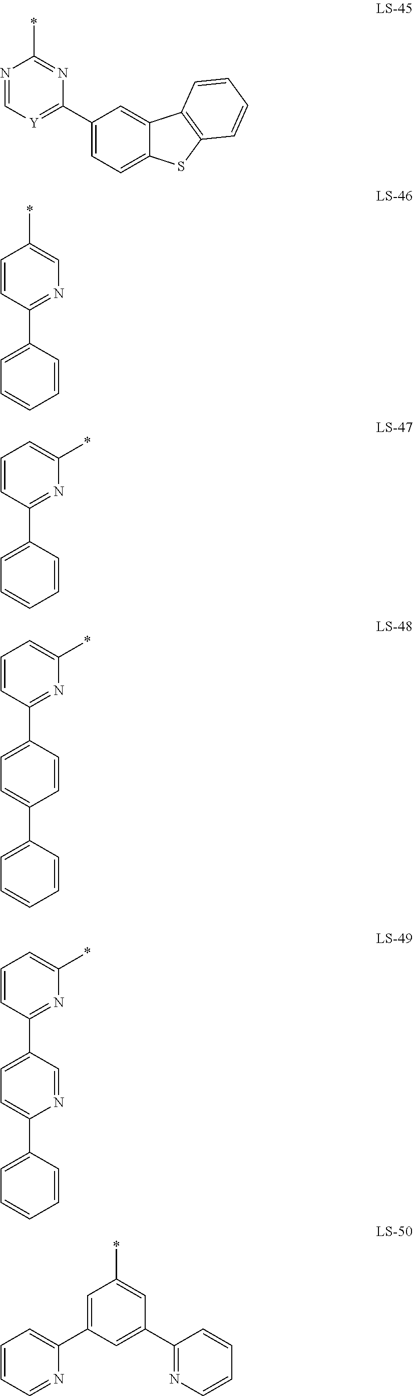

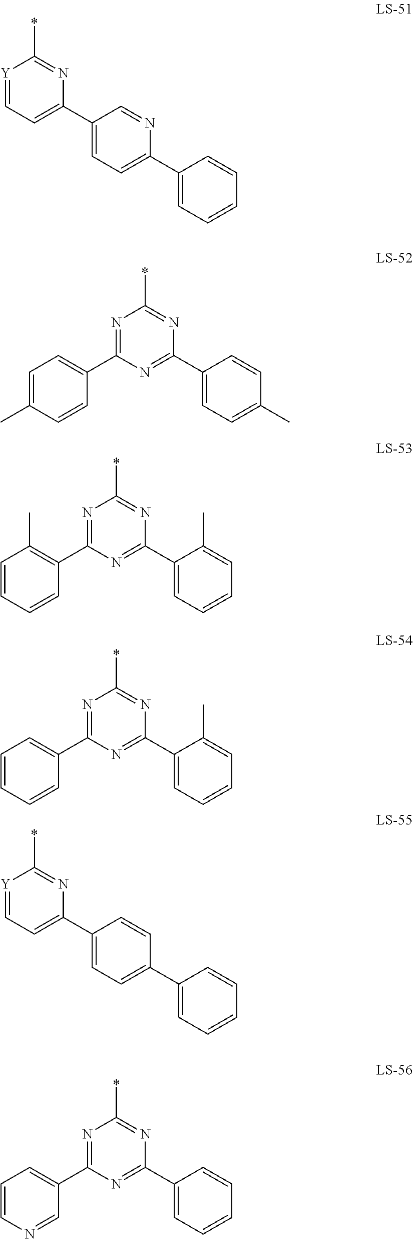

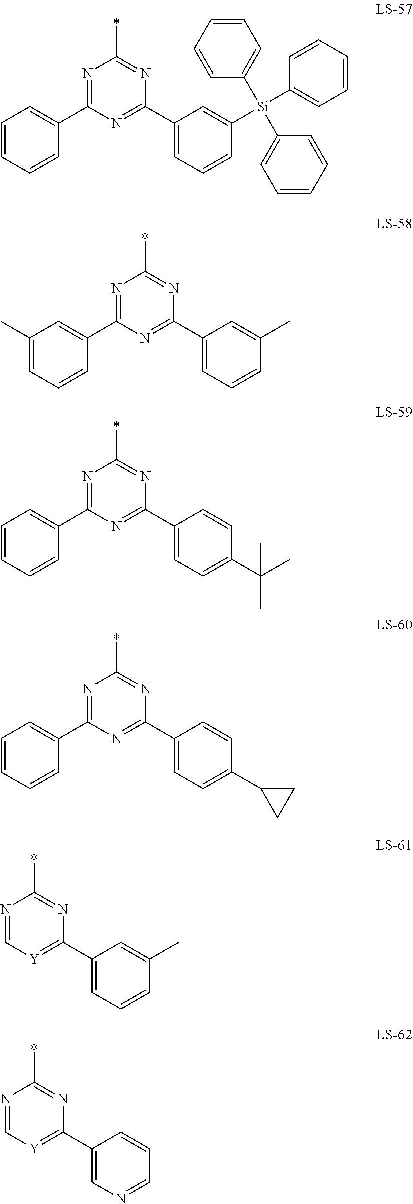

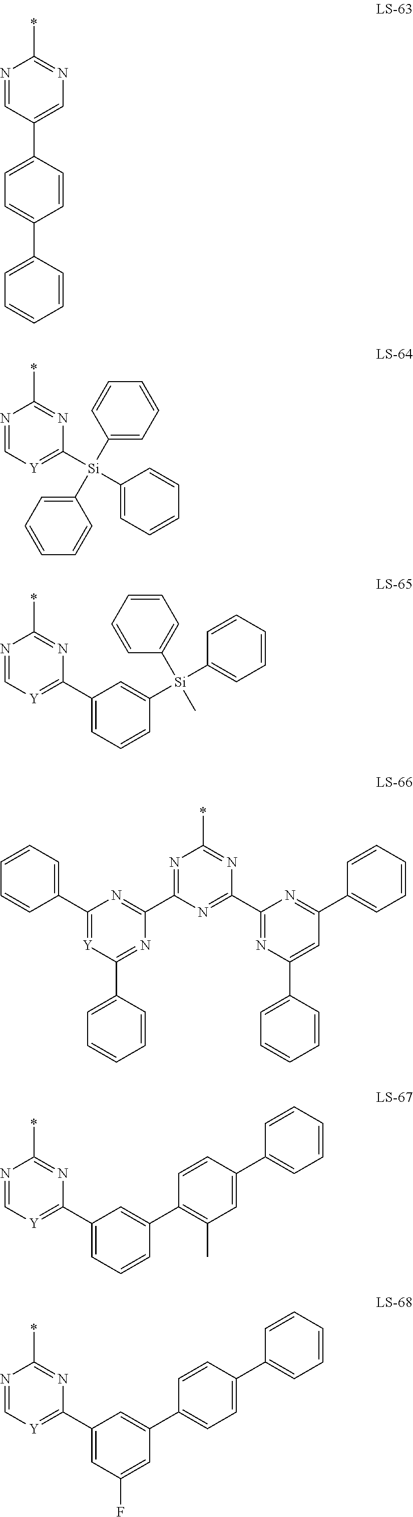

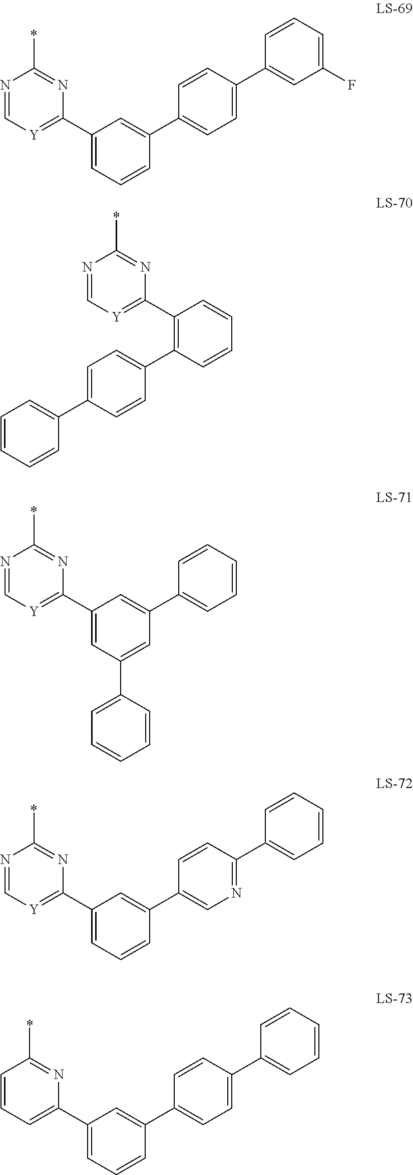

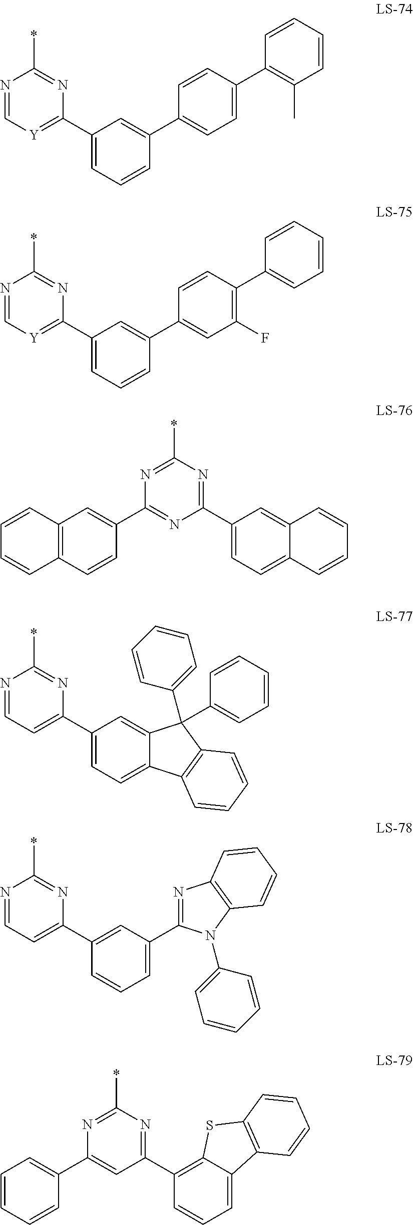

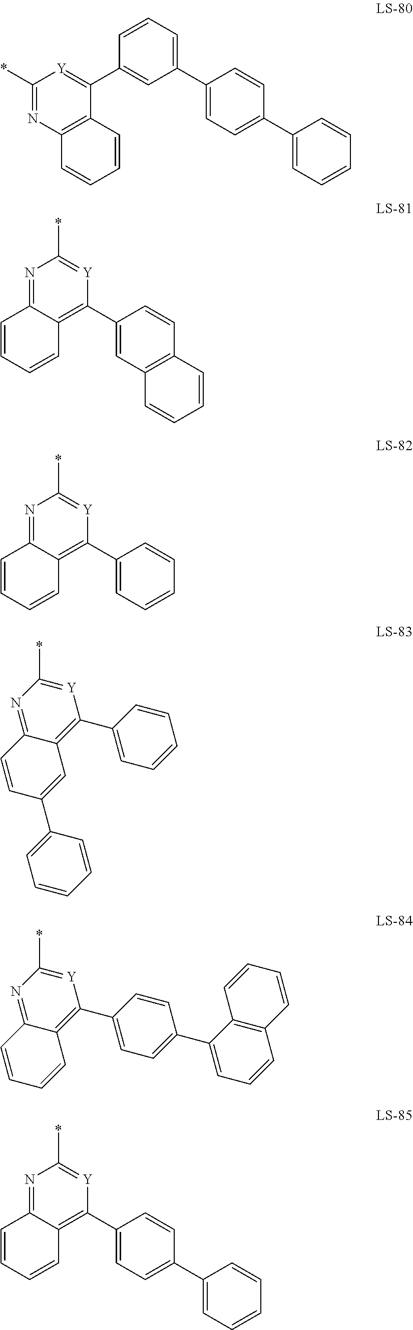

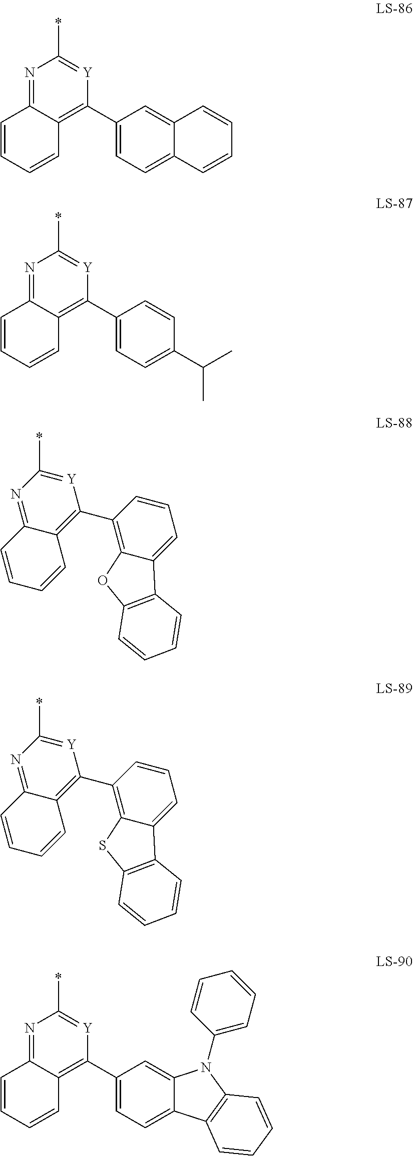

7. The combination according to claim 1, wherein the compound represented by formula 1 is selected from the group consisting of:

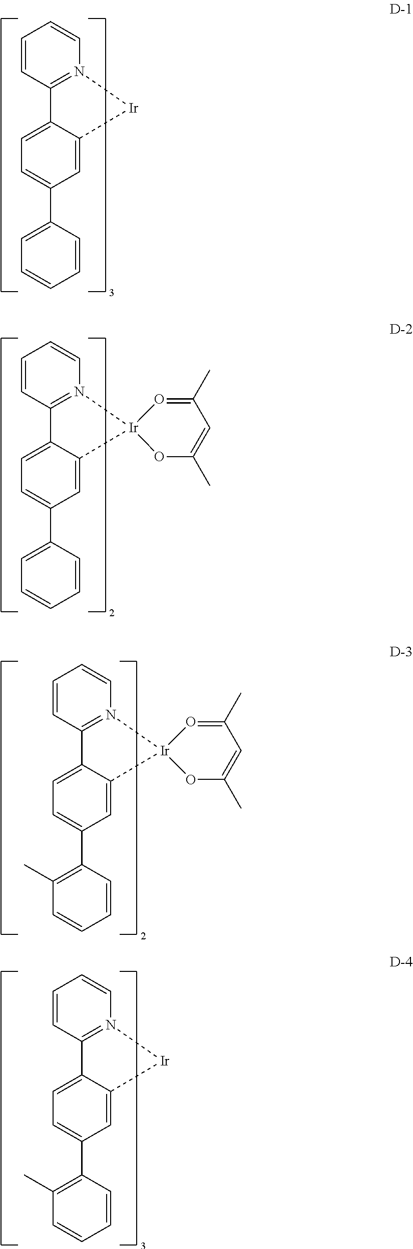

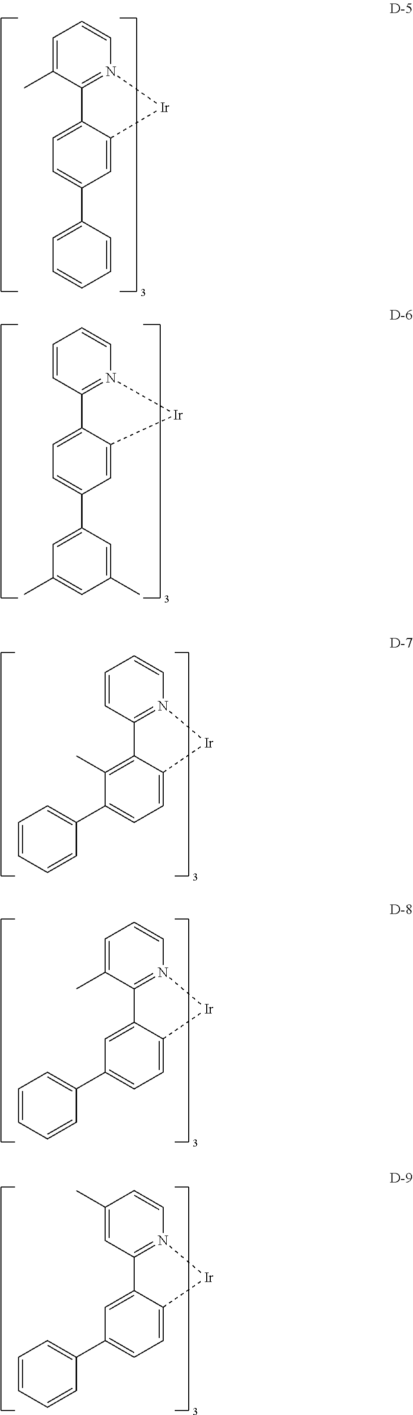

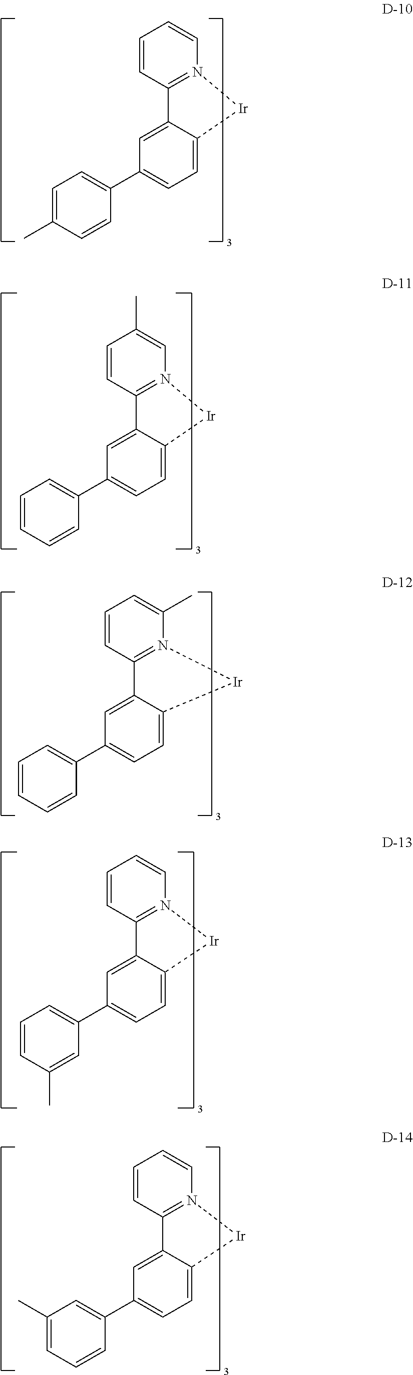

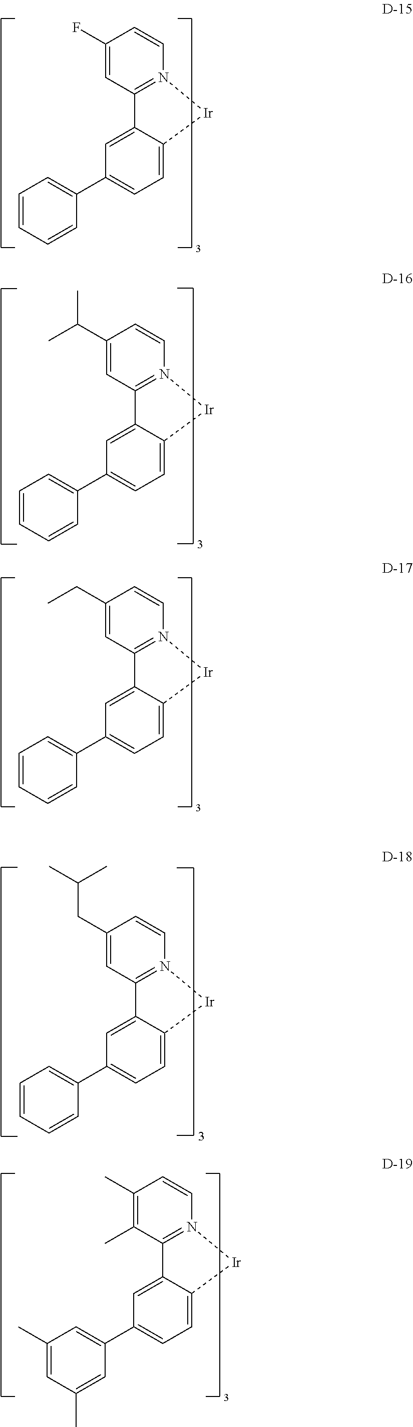

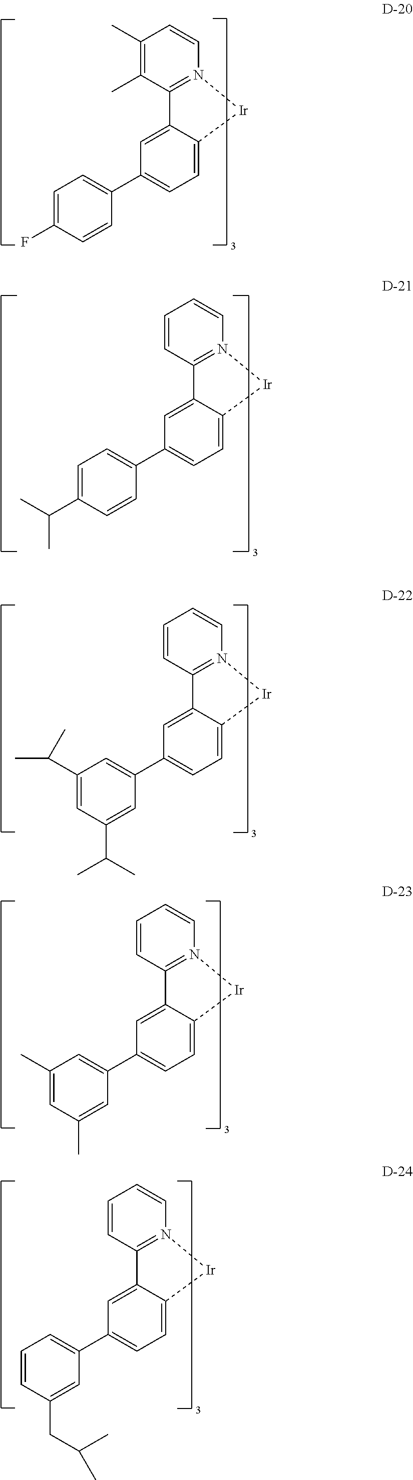

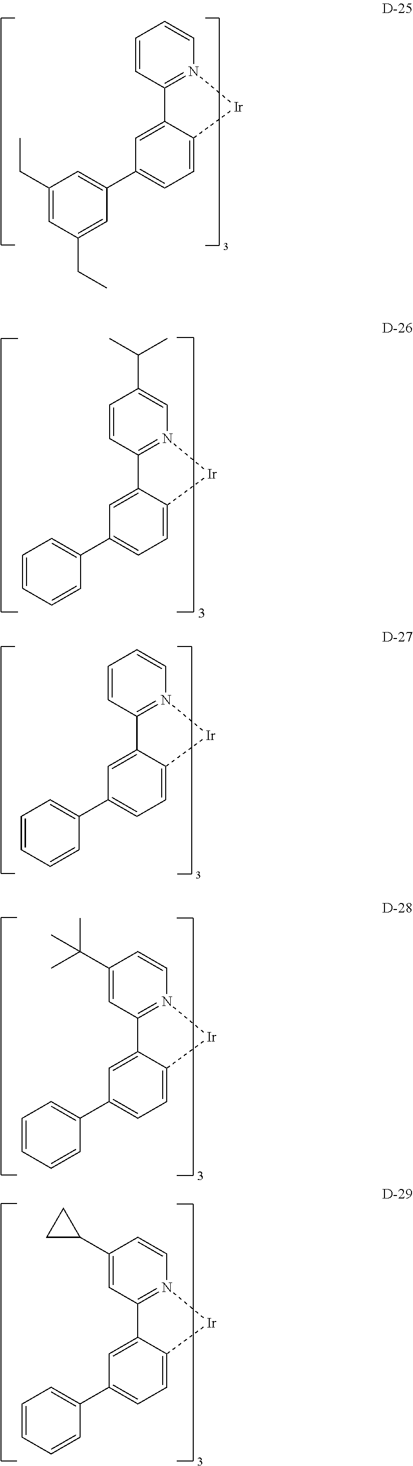

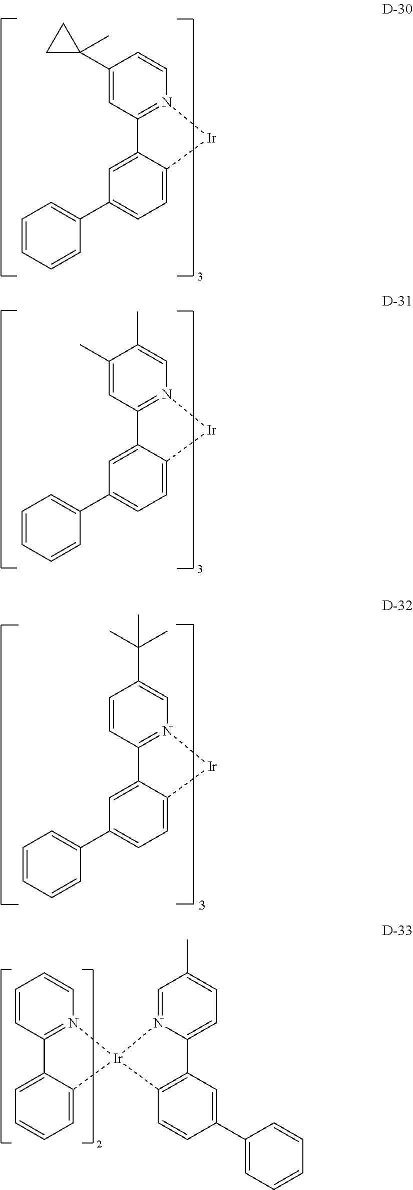

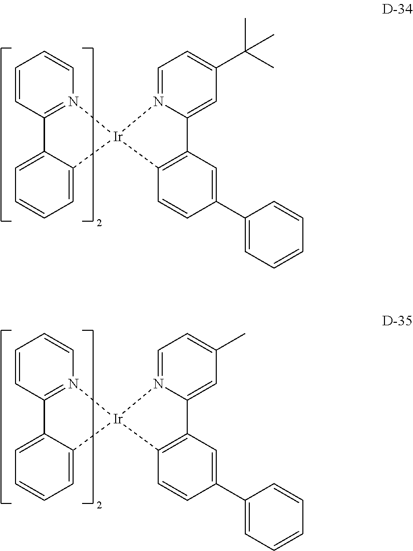

8. The combination according to claim 1, wherein the compound represented by formula 2 is selected from the group consisting of:

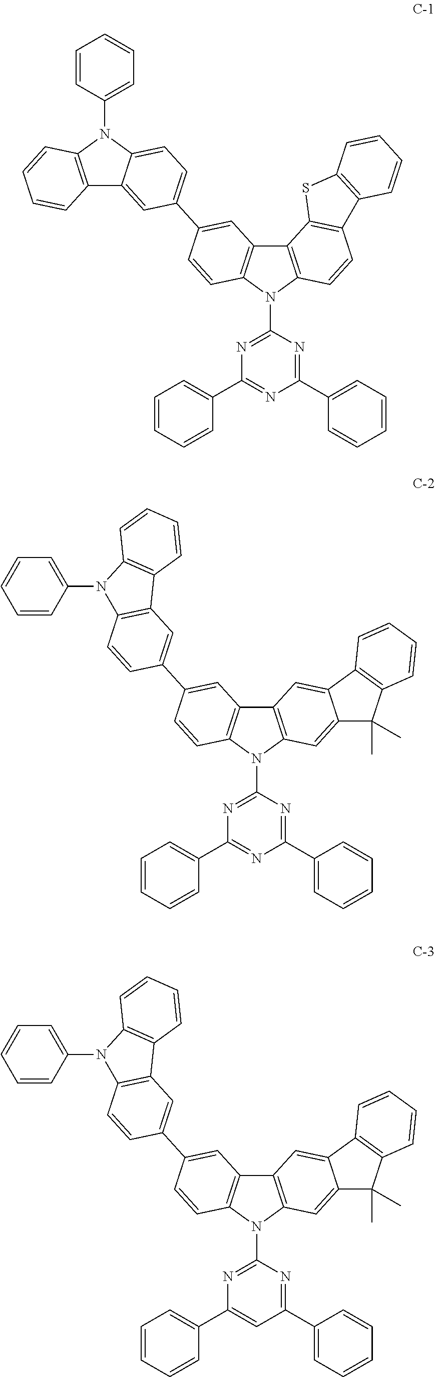 [SMILES: c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1 ; CC1(C)c2ccccc2-c2cc3c4cc(-c5ccc6c(c5)c5ccccc5n6-c5ccccc5)ccc4n(-c4nc(-c5ccccc5)nc(-c5ccccc5)n4)c3cc21 ; CC1(C)c2ccccc2-c2cc3c4cc(-c5ccc6c(c5)c5ccccc5n6-c5ccccc5)ccc4n(-c4nc(-c5ccccc5)cc(-c5ccccc5)n4)c3cc21]

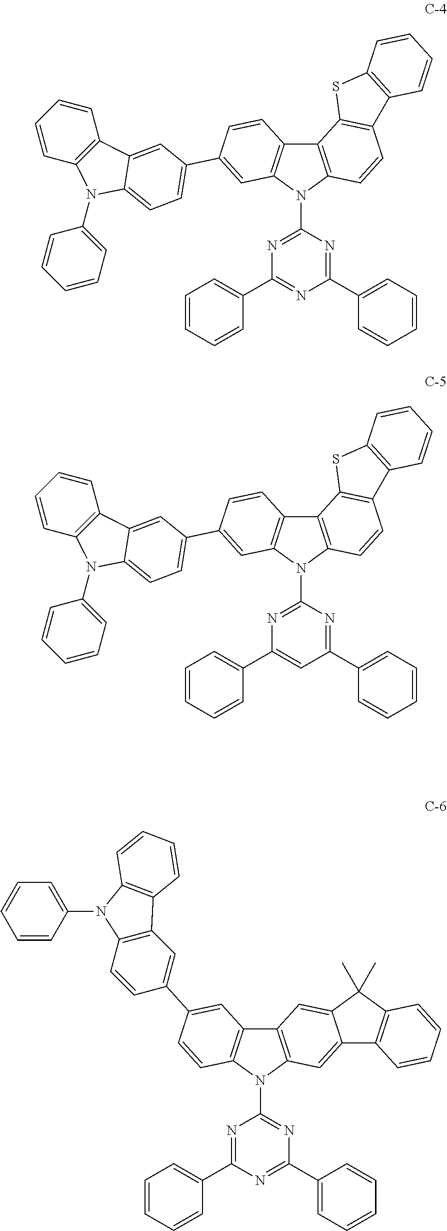 [SMILES: c1ccc(-c2nc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1 ; CC1(C)c2ccccc2-c2cc3c(cc21)c1cc(-c2ccc4c(c2)c2ccccc2n4-c2ccccc2)ccc1n3-c1nc(-c2ccccc2)nc(-c2ccccc2)n1]

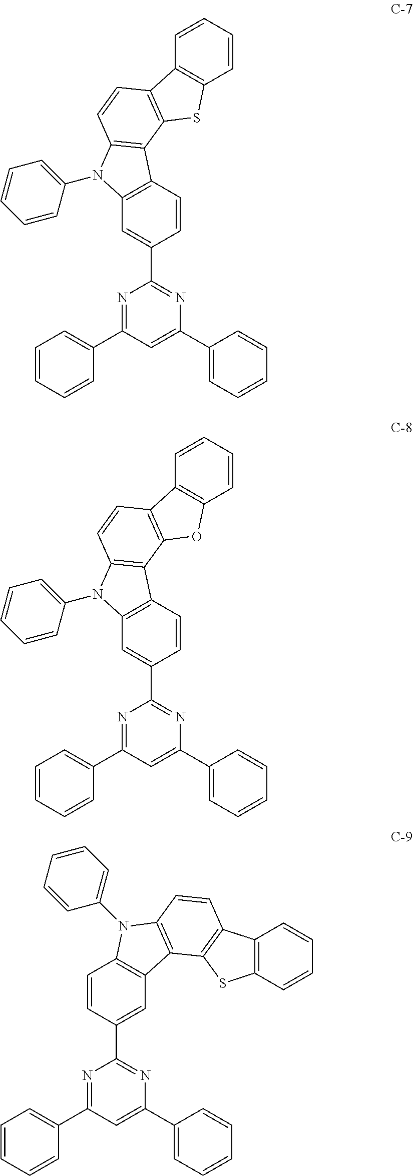

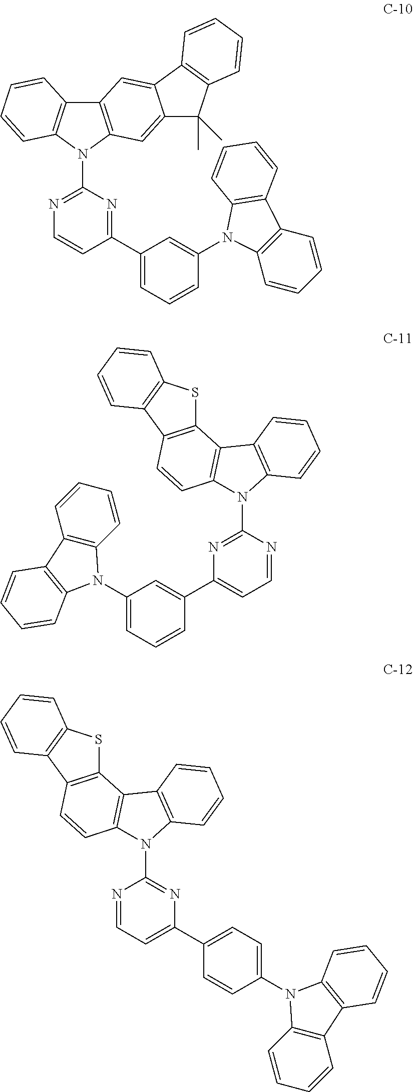 [SMILES: CC1(C)c2ccccc2-c2cc3c4ccccc4n(-c4nccc(-c5cccc(-n6c7ccccc7c7ccccc76)c5)n4)c3cc21 ; c1cc(-c2ccnc(-n3c4ccccc4c4c5sc6ccccc6c5ccc43)n2)cc(-n2c3ccccc3c3ccccc32)c1 ; c1ccc2c(c1)sc1c2ccc2c1c1ccccc1n2-c1nccc(-c2ccc(-n3c4ccccc4c4ccccc43)cc2)n1]

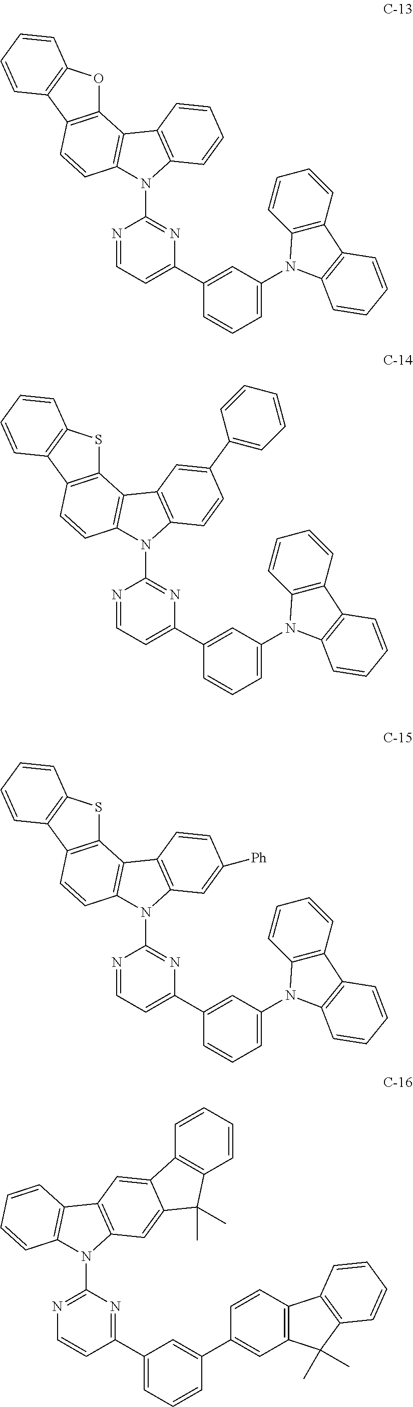 [SMILES: c1cc(-c2ccnc(-n3c4ccccc4c4c5oc6ccccc6c5ccc43)n2)cc(-n2c3ccccc3c3ccccc32)c1 ; c1ccc(-c2ccc3c(c2)c2c4sc5ccccc5c4ccc2n3-c2nccc(-c3cccc(-n4c5ccccc5c5ccccc54)c3)n2)cc1 ; c1ccc(-c2ccc3c4c5sc6ccccc6c5ccc4n(-c4nccc(-c5cccc(-n6c7ccccc7c7ccccc76)c5)n4)c3c2)cc1 ; CC1(C)c2ccccc2-c2ccc(-c3cccc(-c4ccnc(-n5c6ccccc6c6cc7c(cc65)C(C)(C)c5ccccc5-7)n4)c3)cc21]

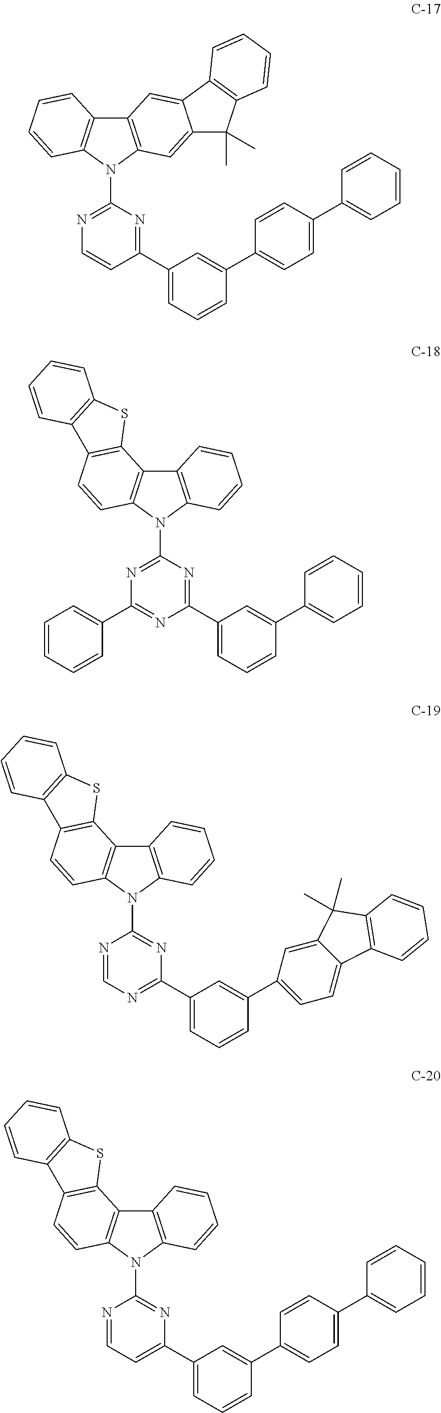 [SMILES: CC1(C)c2ccccc2-c2cc3c4ccccc4n(-c4nccc(-c5cccc(-c6ccc(-c7ccccc7)cc6)c5)n4)c3cc21 ; c1ccc(-c2cccc(-c3nc(-c4ccccc4)nc(-n4c5ccccc5c5c6sc7ccccc7c6ccc54)n3)c2)cc1 ; CC1(C)c2ccccc2-c2ccc(-c3cccc(-c4ncnc(-n5c6ccccc6c6c7sc8ccccc8c7ccc65)n4)c3)cc21 ; c1ccc(-c2ccc(-c3cccc(-c4ccnc(-n5c6ccccc6c6c7sc8ccccc8c7ccc65)n4)c3)cc2)cc1]

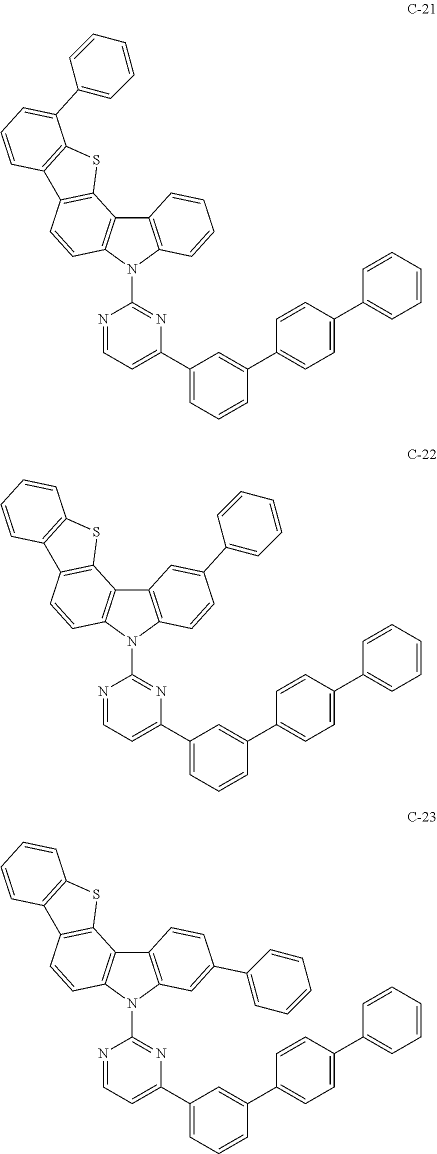 [SMILES: c1ccc(-c2ccc(-c3cccc(-c4ccnc(-n5c6ccccc6c6c7sc8c(-c9ccccc9)cccc8c7ccc65)n4)c3)cc2)cc1 ; c1ccc(-c2ccc(-c3cccc(-c4ccnc(-n5c6ccc(-c7ccccc7)cc6c6c7sc8ccccc8c7ccc65)n4)c3)cc2)cc1 ; c1ccc(-c2ccc(-c3cccc(-c4ccnc(-n5c6cc(-c7ccccc7)ccc6c6c7sc8ccccc8c7ccc65)n4)c3)cc2)cc1]

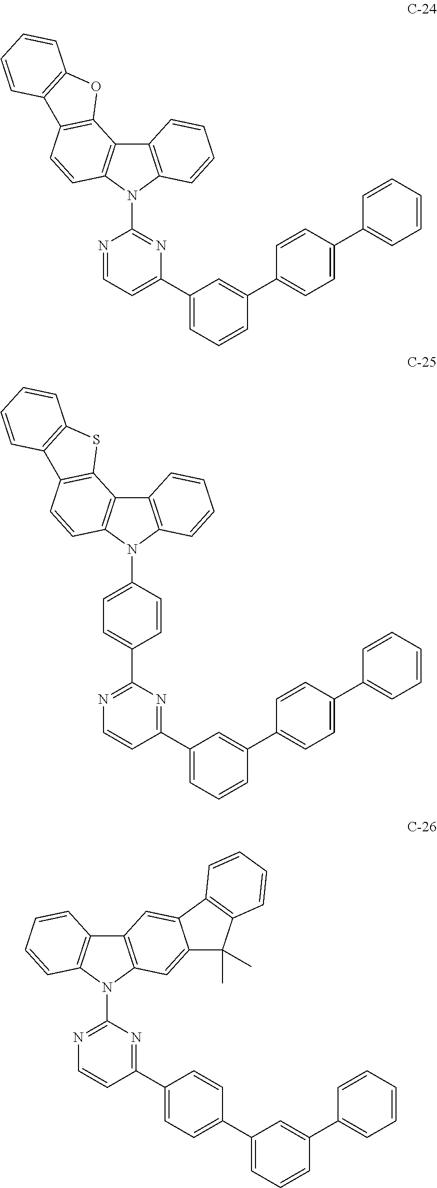 [SMILES: c1ccc(-c2ccc(-c3cccc(-c4ccnc(-n5c6ccccc6c6c7oc8ccccc8c7ccc65)n4)c3)cc2)cc1 ; c1ccc(-c2ccc(-c3cccc(-c4ccnc(-c5ccc(-n6c7ccccc7c7c8sc9ccccc9c8ccc76)cc5)n4)c3)cc2)cc1 ; CC1(C)c2ccccc2-c2cc3c4ccccc4n(-c4nccc(-c5ccc(-c6cccc(-c7ccccc7)c6)cc5)n4)c3cc21]

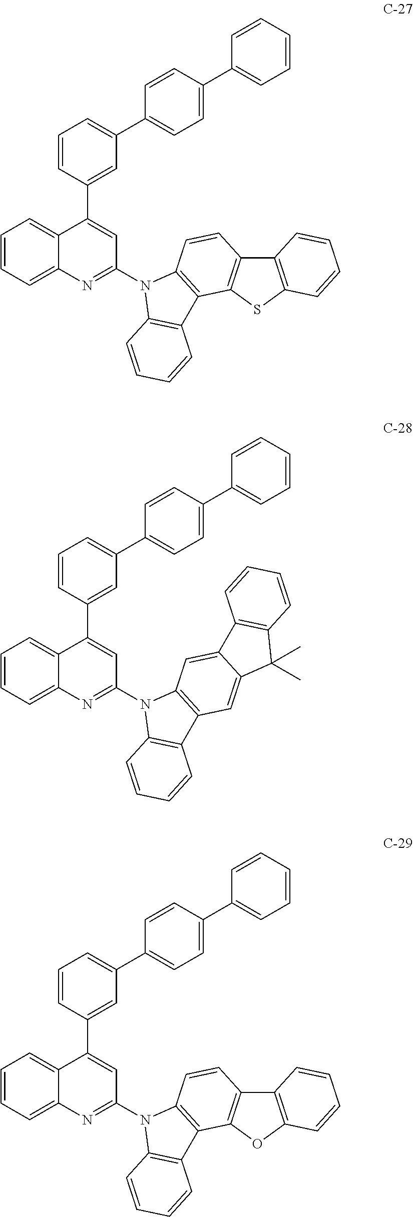 [SMILES: c1ccc(-c2ccc(-c3cccc(-c4cc(-n5c6ccccc6c6c7sc8ccccc8c7ccc65)nc5ccccc45)c3)cc2)cc1 ; CC1(C)c2ccccc2-c2cc3c(cc21)c1ccccc1n3-c1cc(-c2cccc(-c3ccc(-c4ccccc4)cc3)c2)c2ccccc2n1 ; c1ccc(-c2ccc(-c3cccc(-c4cc(-n5c6ccccc6c6c7oc8ccccc8c7ccc65)nc5ccccc45)c3)cc2)cc1]

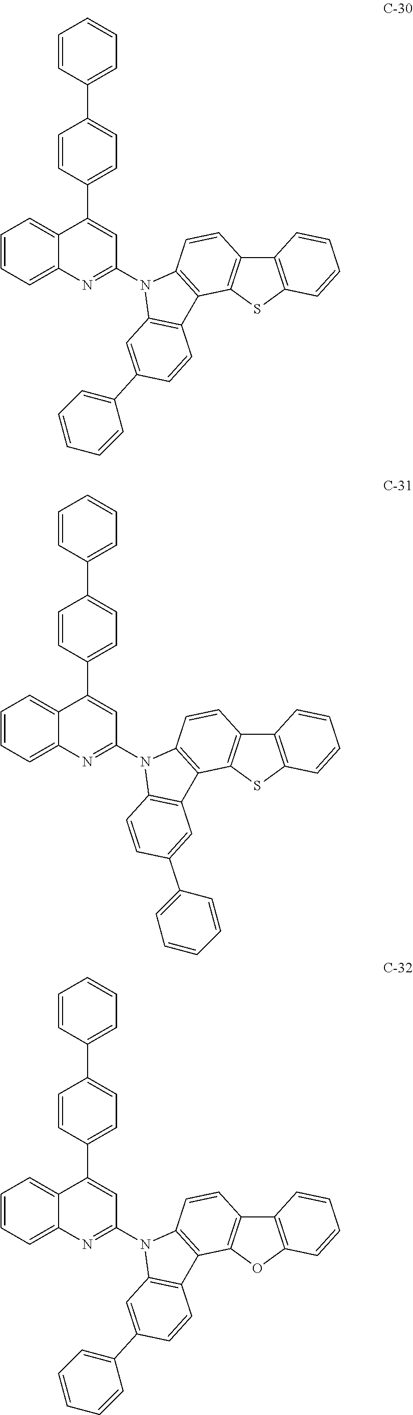

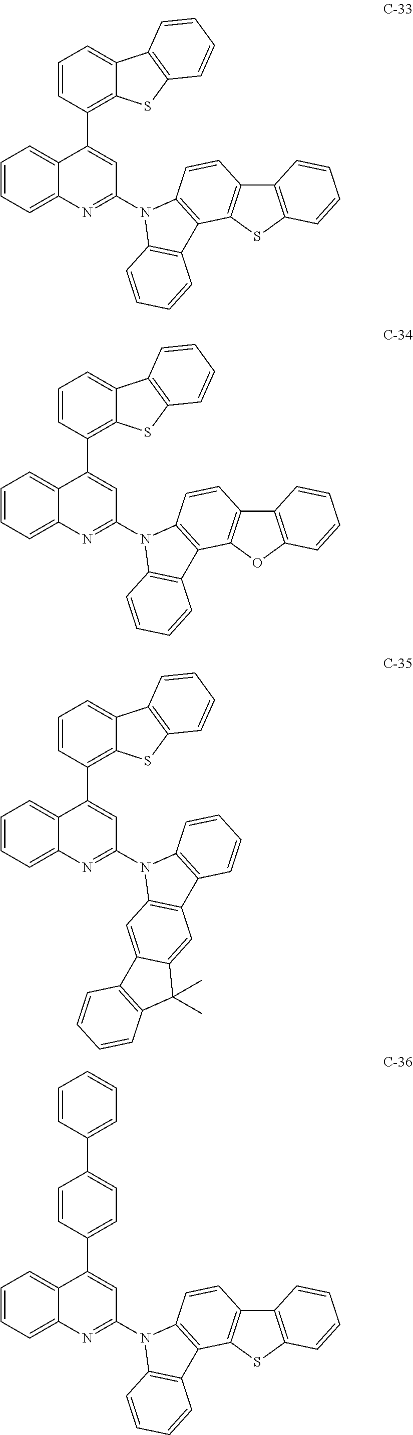

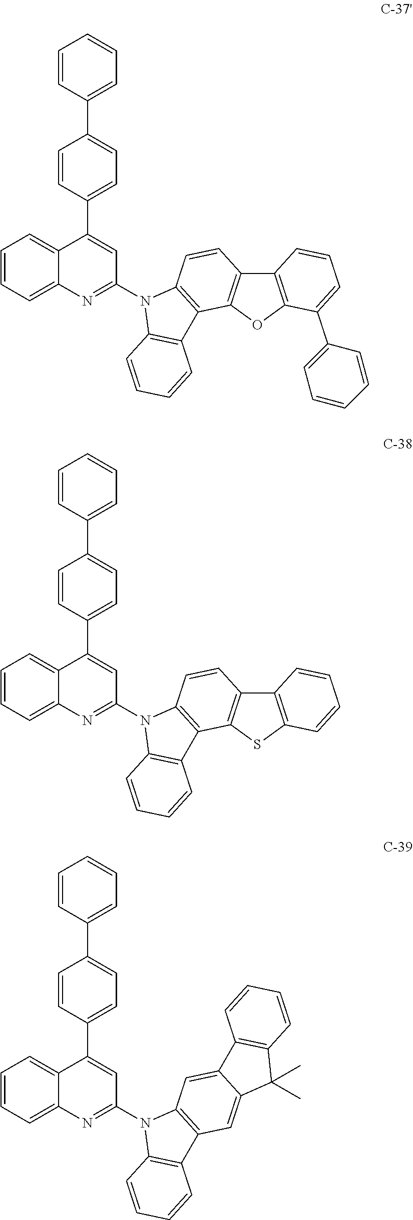 [SMILES: c1ccc(-c2ccc(-c3cc(-n4c5ccccc5c5c6oc7c(-c8ccccc8)cccc7c6ccc54)nc4ccccc34)cc2)cc1 ; c1ccc(-c2ccc(-c3cc(-n4c5ccccc5c5c6sc7ccccc7c6ccc54)nc4ccccc34)cc2)cc1 ; CC1(C)c2ccccc2-c2cc3c(cc21)c1ccccc1n3-c1cc(-c2ccc(-c3ccccc3)cc2)c2ccccc2n1]

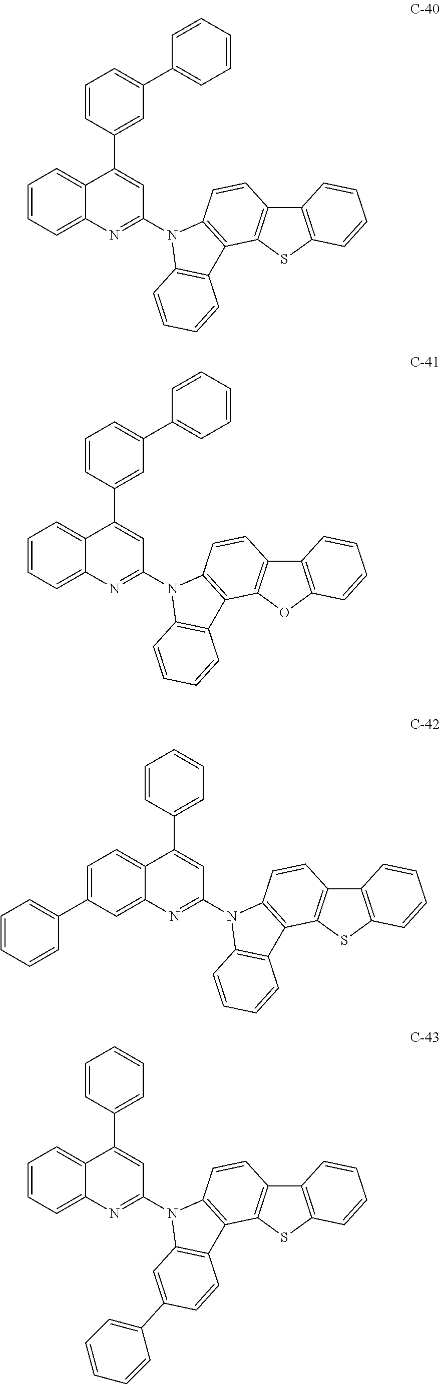

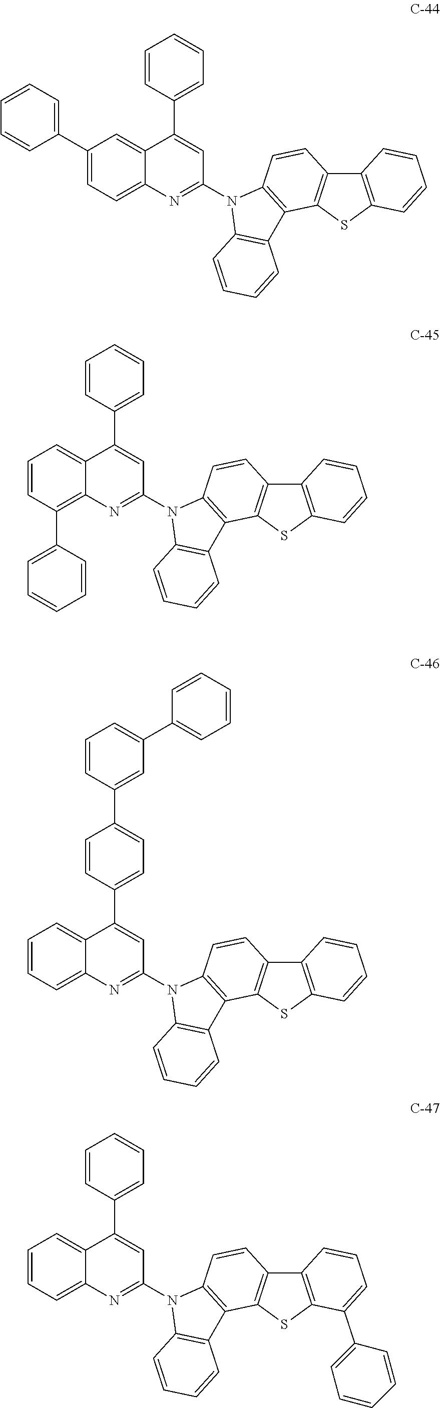 [SMILES: c1ccc(-c2ccc3nc(-n4c5ccccc5c5c6sc7ccccc7c6ccc54)cc(-c4ccccc4)c3c2)cc1 ; c1ccc(-c2cc(-n3c4ccccc4c4c5sc6ccccc6c5ccc43)nc3c(-c4ccccc4)cccc23)cc1 ; c1ccc(-c2cccc(-c3ccc(-c4cc(-n5c6ccccc6c6c7sc8ccccc8c7ccc65)nc5ccccc45)cc3)c2)cc1 ; c1ccc(-c2cc(-n3c4ccccc4c4c5sc6c(-c7ccccc7)cccc6c5ccc43)nc3ccccc23)cc1]

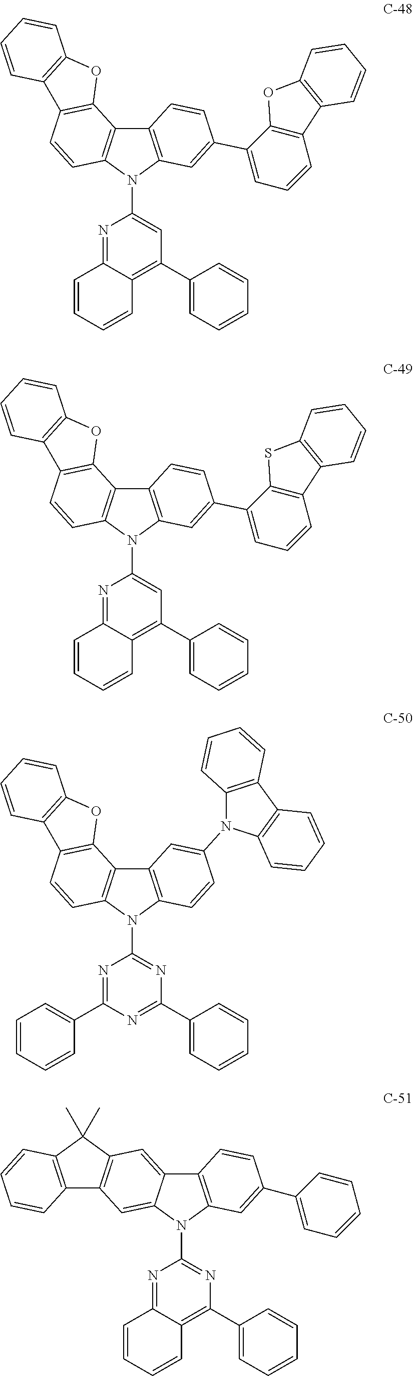

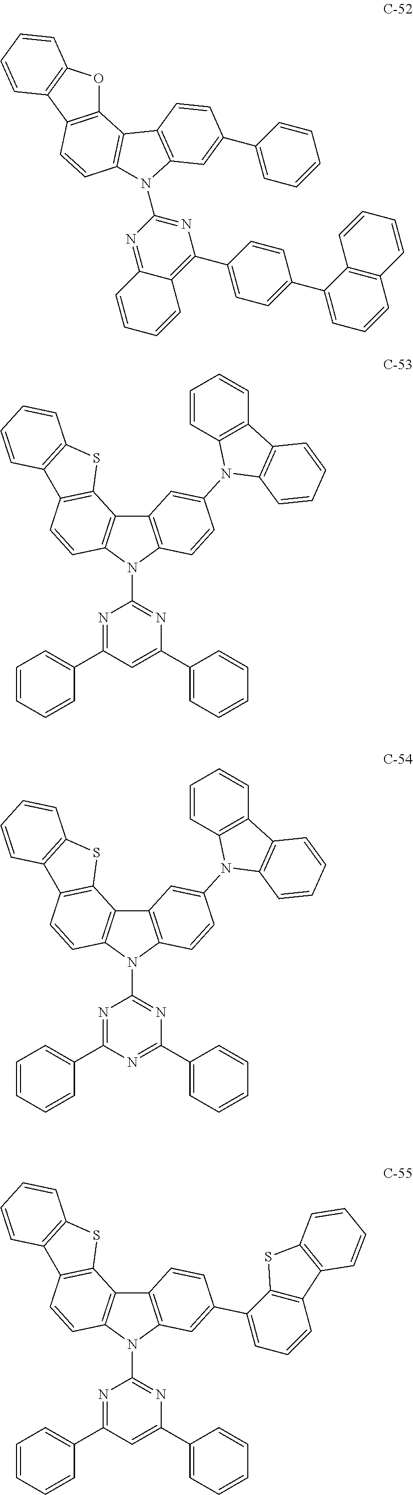 [SMILES: c1ccc(-c2ccc3c4c5oc6ccccc6c5ccc4n(-c4nc(-c5ccc(-c6cccc7ccccc67)cc5)c5ccccc5n4)c3c2)cc1 ; c1ccc(-c2cc(-c3ccccc3)nc(-n3c4ccc(-n5c6ccccc6c6ccccc65)cc4c4c5sc6ccccc6c5ccc43)n2)cc1 ; c1ccc(-c2nc(-c3ccccc3)nc(-n3c4ccc(-n5c6ccccc6c6ccccc65)cc4c4c5sc6ccccc6c5ccc43)n2)cc1 ; c1ccc(-c2cc(-c3ccccc3)nc(-n3c4cc(-c5cccc6c5sc5ccccc56)ccc4c4c5sc6ccccc6c5ccc43)n2)cc1]

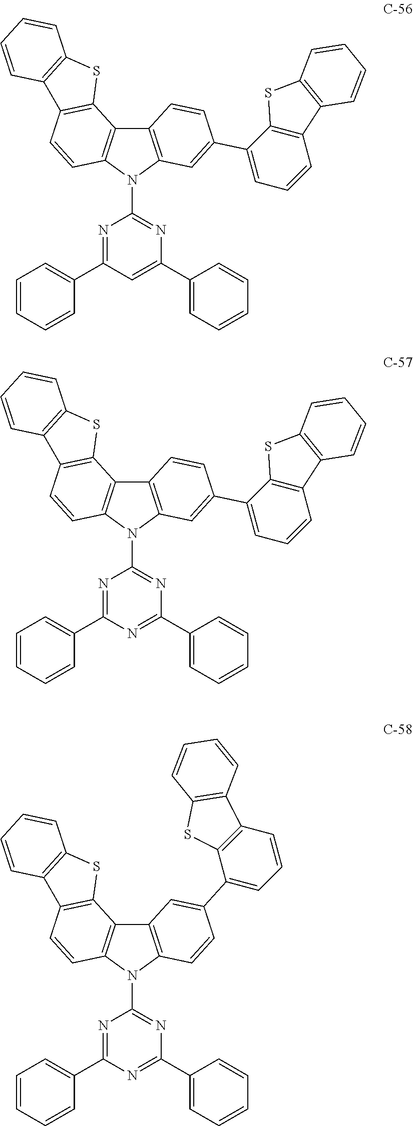 [SMILES: c1ccc(-c2cc(-c3ccccc3)nc(-n3c4cc(-c5cccc6c5sc5ccccc56)ccc4c4c5sc6ccccc6c5ccc43)n2)cc1 ; c1ccc(-c2nc(-c3ccccc3)nc(-n3c4cc(-c5cccc6c5sc5ccccc56)ccc4c4c5sc6ccccc6c5ccc43)n2)cc1 ; c1ccc(-c2nc(-c3ccccc3)nc(-n3c4ccc(-c5cccc6c5sc5ccccc56)cc4c4c5sc6ccccc6c5ccc43)n2)cc1]

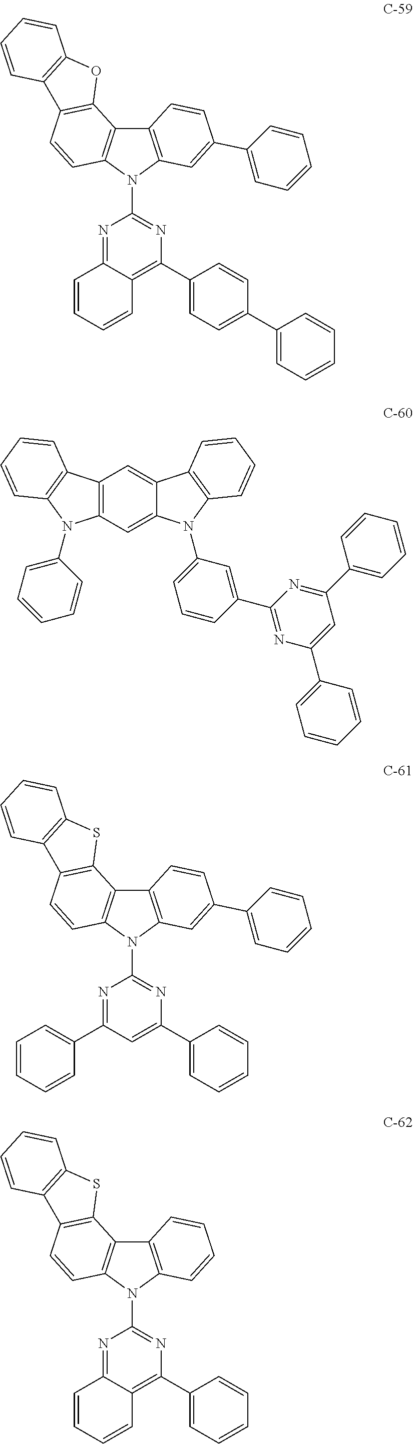

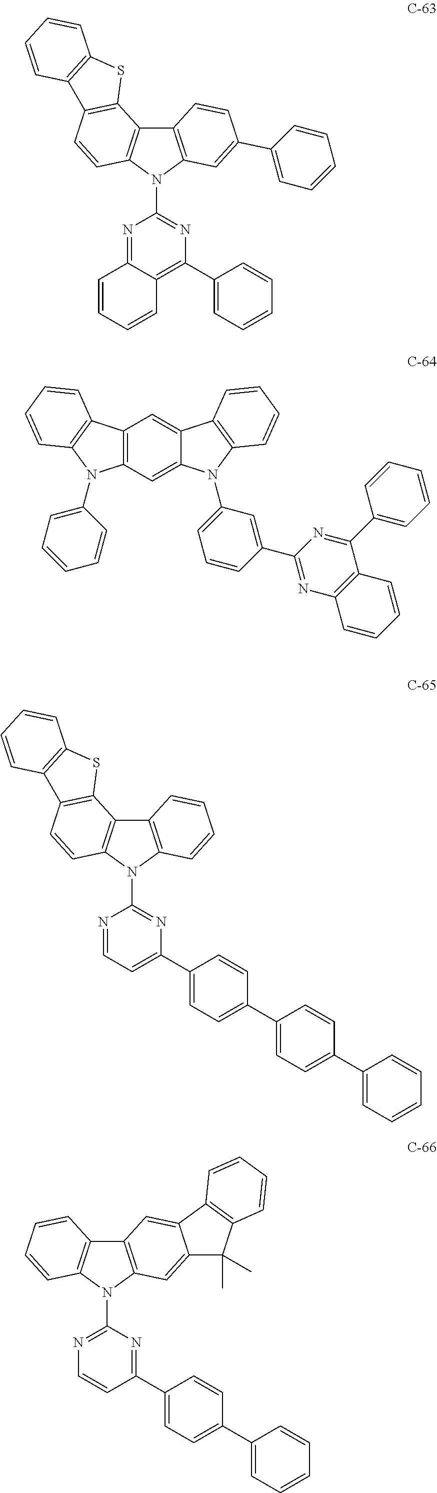 [SMILES: c1ccc(-c2ccc3c4c5sc6ccccc6c5ccc4n(-c4nc(-c5ccccc5)c5ccccc5n4)c3c2)cc1 ; c1ccc(-c2nc(-c3cccc(-n4c5ccccc5c5cc6c7ccccc7n(-c7ccccc7)c6cc54)c3)nc3ccccc23)cc1 ; c1ccc(-c2ccc(-c3ccc(-c4ccnc(-n5c6ccccc6c6c7sc8ccccc8c7ccc65)n4)cc3)cc2)cc1 ; CC1(C)c2ccccc2-c2cc3c4ccccc4n(-c4nccc(-c5ccc(-c6ccccc6)cc5)n4)c3cc21]

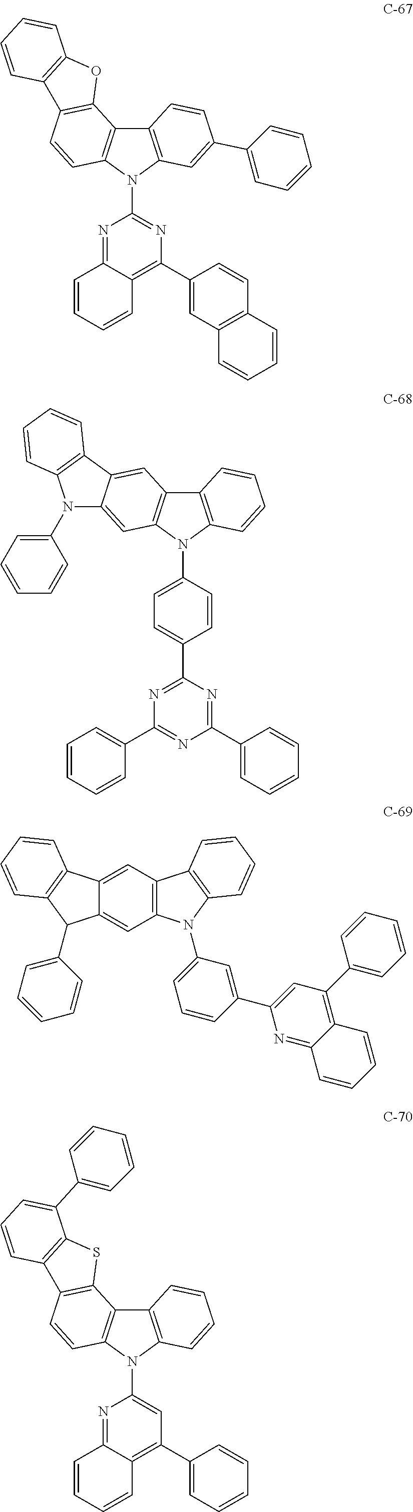 [SMILES: c1ccc(-c2ccc3c4c5oc6ccccc6c5ccc4n(-c4nc(-c5ccc6ccccc6c5)c5ccccc5n4)c3c2)cc1 ; c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2cc(-c3cccc(-n4c5ccccc5c5cc6c(cc54)C(c4ccccc4)c4ccccc4-6)c3)nc3ccccc23)cc1 ; c1ccc(-c2cc(-n3c4ccccc4c4c5sc6c(-c7ccccc7)cccc6c5ccc43)nc3ccccc23)cc1]

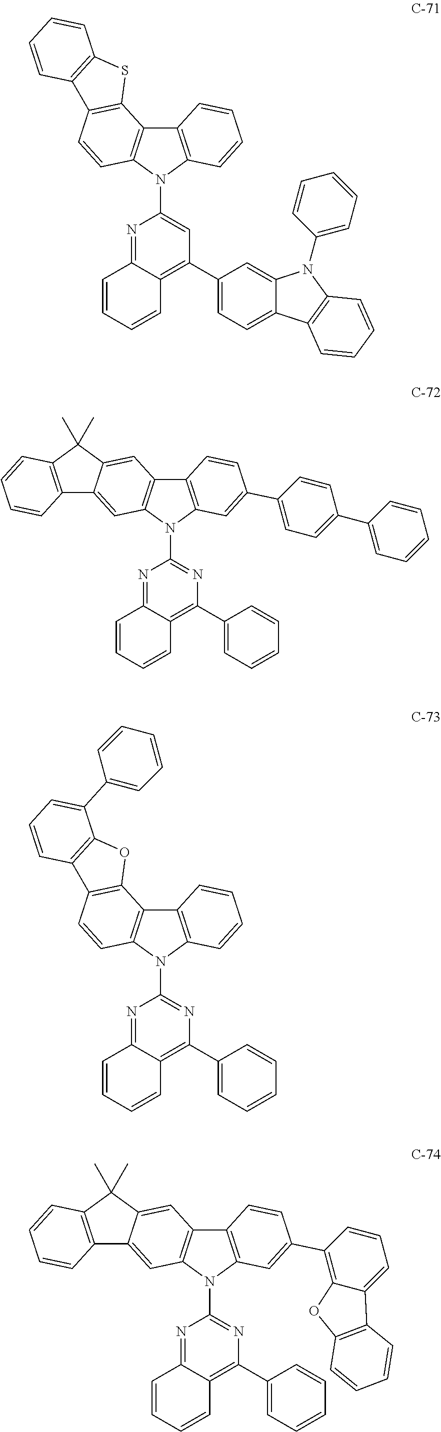 [SMILES: c1ccc(-n2c3ccccc3c3ccc(-c4cc(-n5c6ccccc6c6c7sc8ccccc8c7ccc65)nc5ccccc45)cc32)cc1 ; CC1(C)c2ccccc2-c2cc3c(cc21)c1ccc(-c2ccc(-c4ccccc4)cc2)cc1n3-c1nc(-c2ccccc2)c2ccccc2n1 ; c1ccc(-c2nc(-n3c4ccccc4c4c5oc6c(-c7ccccc7)cccc6c5ccc43)nc3ccccc23)cc1 ; CC1(C)c2ccccc2-c2cc3c(cc21)c1ccc(-c2cccc4c2oc2ccccc24)cc1n3-c1nc(-c2ccccc2)c2ccccc2n1]

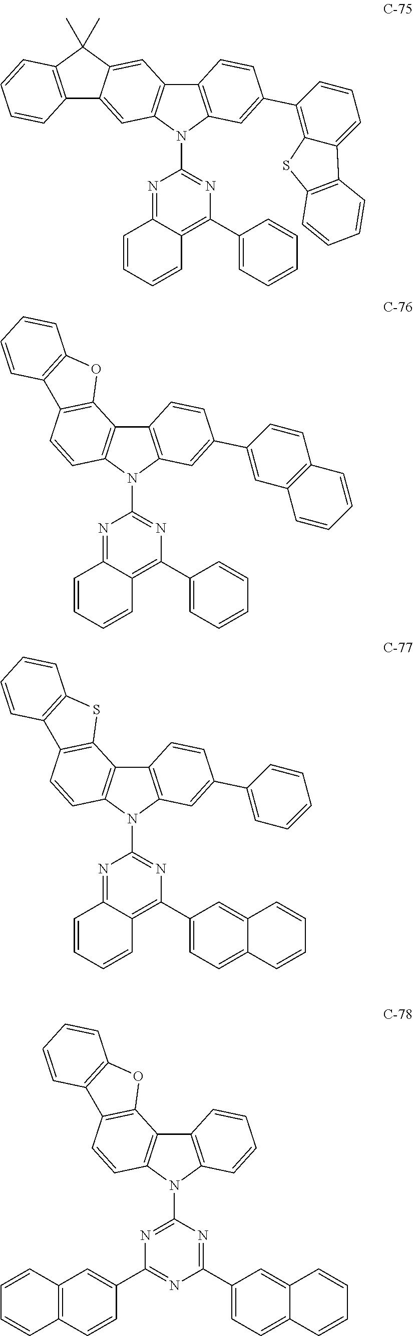 [SMILES: CC1(C)c2ccccc2-c2cc3c(cc21)c1ccc(-c2cccc4c2sc2ccccc24)cc1n3-c1nc(-c2ccccc2)c2ccccc2n1 ; c1ccc(-c2nc(-n3c4cc(-c5ccc6ccccc6c5)ccc4c4c5oc6ccccc6c5ccc43)nc3ccccc23)cc1 ; c1ccc(-c2ccc3c4c5sc6ccccc6c5ccc4n(-c4nc(-c5ccc6ccccc6c5)c5ccccc5n4)c3c2)cc1 ; c1ccc2cc(-c3nc(-c4ccc5ccccc5c4)nc(-n4c5ccccc5c5c6oc7ccccc7c6ccc54)n3)ccc2c1]

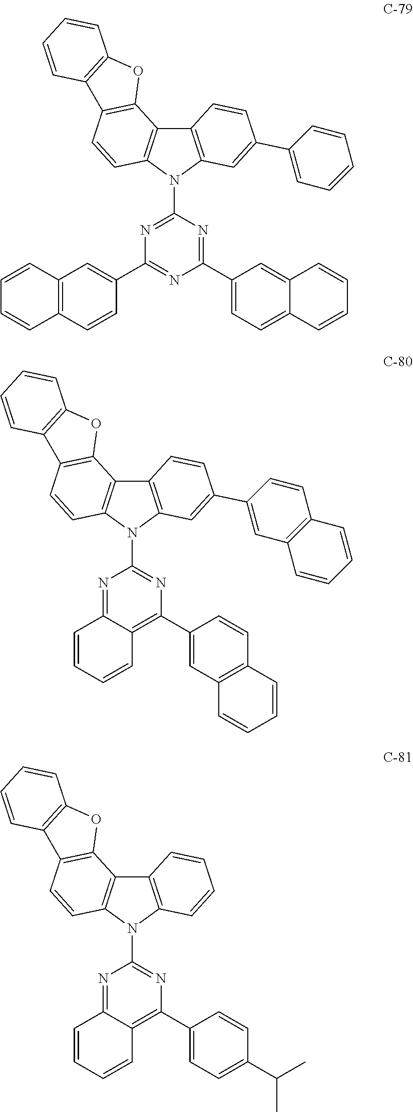

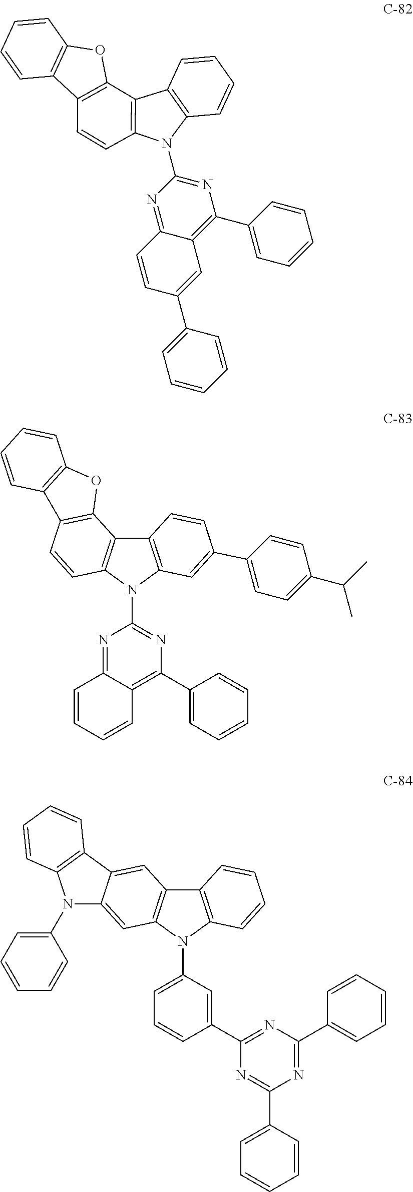 [SMILES: c1ccc(-c2ccc3nc(-n4c5ccccc5c5c6oc7ccccc7c6ccc54)nc(-c4ccccc4)c3c2)cc1 ; CC(C)c1ccc(-c2ccc3c4c5oc6ccccc6c5ccc4n(-c4nc(-c5ccccc5)c5ccccc5n4)c3c2)cc1 ; c1ccc(-c2nc(-c3ccccc3)nc(-c3cccc(-n4c5ccccc5c5cc6c7ccccc7n(-c7ccccc7)c6cc54)c3)n2)cc1]

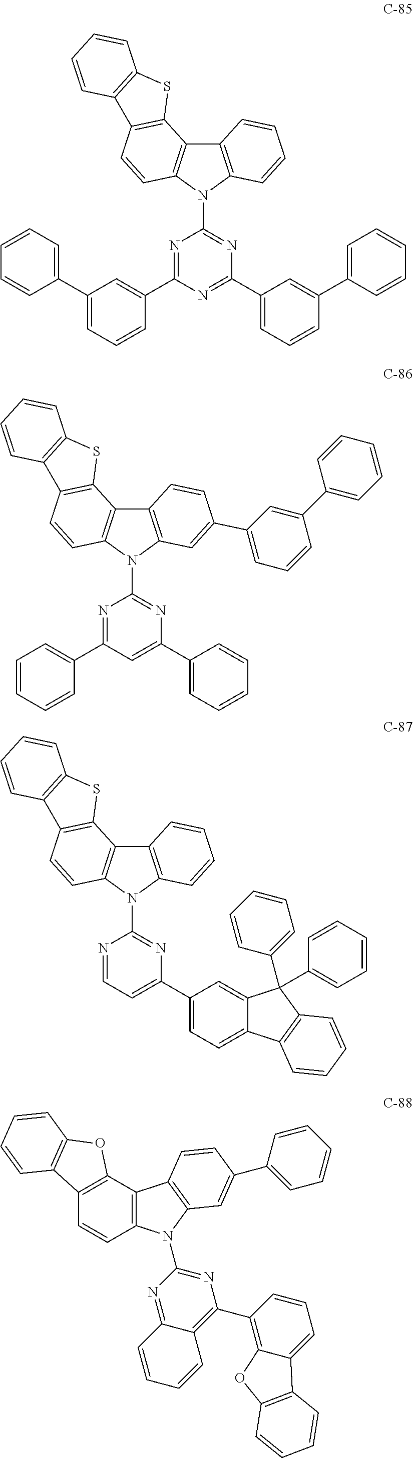

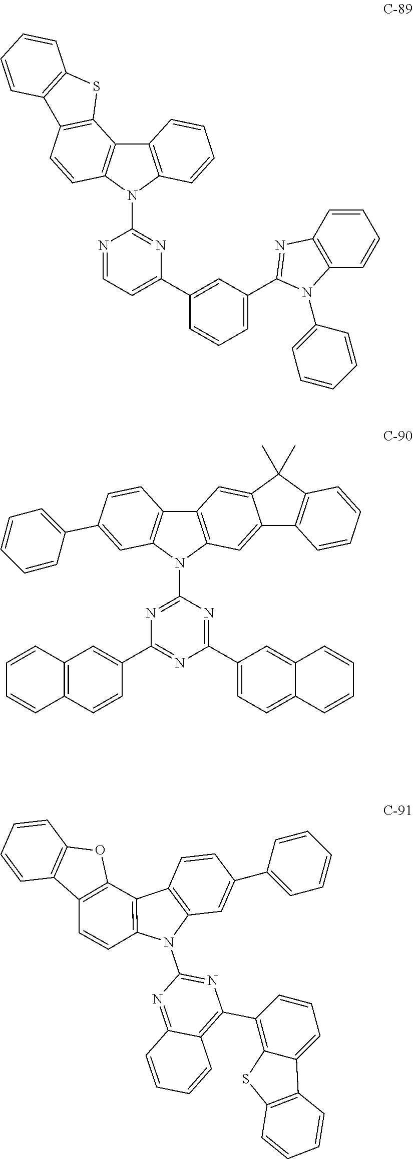 [SMILES: c1ccc(-n2c(-c3cccc(-c4ccnc(-n5c6ccccc6c6c7sc8ccccc8c7ccc65)n4)c3)nc3ccccc32)cc1 ; CC1(C)c2ccccc2-c2cc3c(cc21)c1ccc(-c2ccccc2)cc1n3-c1nc(-c2ccc3ccccc3c2)nc(-c2ccc3ccccc3c2)n1 ; c1ccc(-c2ccc3c4c5oc6ccccc6c5ccc4n(-c4nc(-c5cccc6c5sc5ccccc56)c5ccccc5n4)c3c2)cc1]

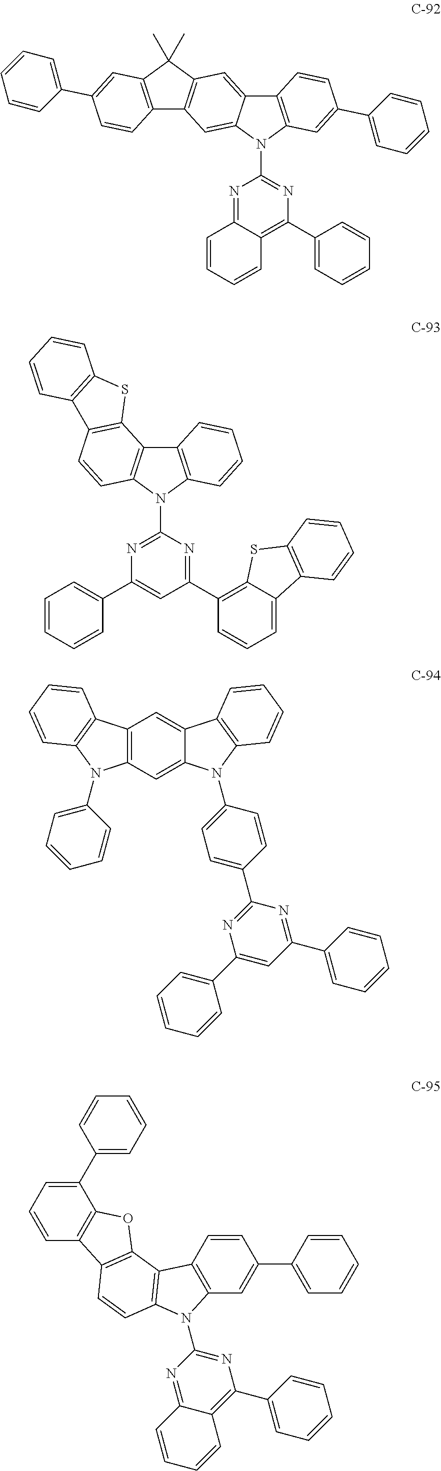

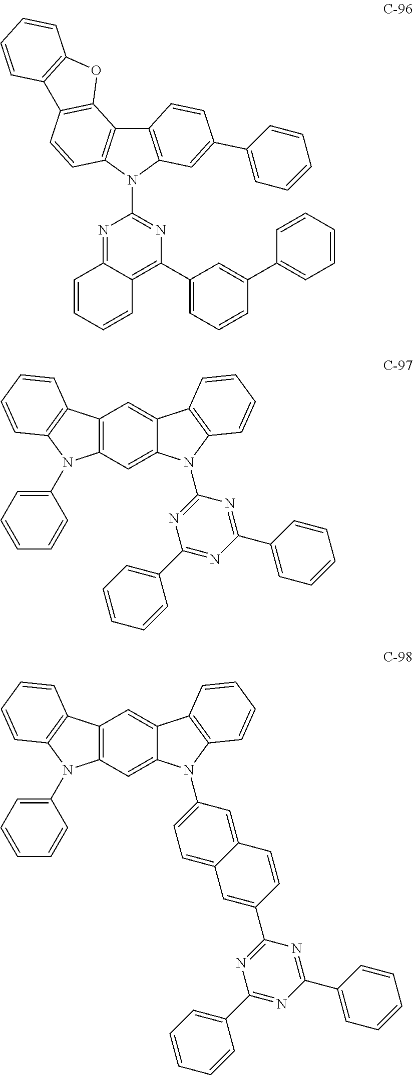 [SMILES: c1ccc(-c2cccc(-c3nc(-n4c5cc(-c6ccccc6)ccc5c5c6oc7ccccc7c6ccc54)nc4ccccc34)c2)cc1 ; c1ccc(-c2nc(-c3ccccc3)nc(-n3c4ccccc4c4cc5c6ccccc6n(-c6ccccc6)c5cc43)n2)cc1 ; c1ccc(-c2nc(-c3ccccc3)nc(-c3ccc4cc(-n5c6ccccc6c6cc7c8ccccc8n(-c8ccccc8)c7cc65)ccc4c3)n2)cc1]

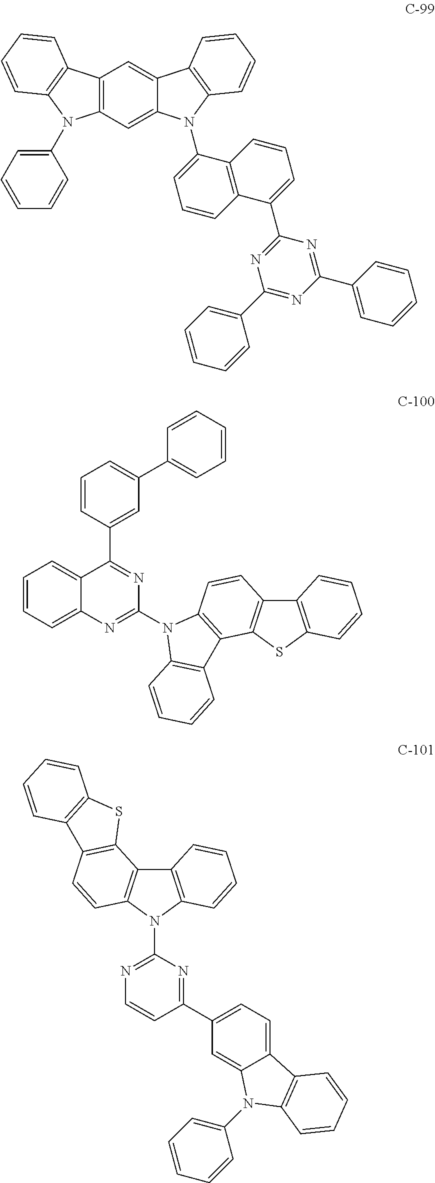 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2cccc(-c3nc(-n4c5ccccc5c5c6sc7ccccc7c6ccc54)nc4ccccc34)c2)cc1 ; c1ccc(-n2c3ccccc3c3ccc(-c4ccnc(-n5c6ccccc6c6c7sc8ccccc8c7ccc65)n4)cc32)cc1]

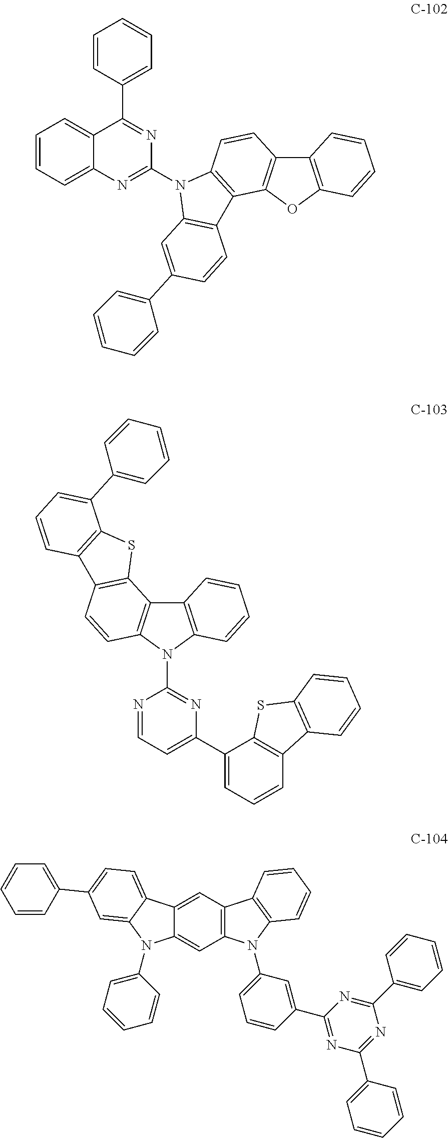 [SMILES: c1ccc(-c2ccc3c4c5oc6ccccc6c5ccc4n(-c4nc(-c5ccccc5)c5ccccc5n4)c3c2)cc1 ; c1ccc(-c2cccc3c2sc2c3ccc3c2c2ccccc2n3-c2nccc(-c3cccc4c3sc3ccccc34)n2)cc1 ; c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1]

9. An organic electroluminescent device which comprises the combination according to claim 1.
